(19) United States
(12) Patent Application Publication
(10) Pub. No.: US 2024/0156765 A1
(43) Pub. Date: May 16, 2024

(54) METHODS OF NEUROPROTECTION AND USES THEREOF

(71) Applicant: UNIVERSITY OF TENNESSEE RESEARCH FOUNDATION, MEMPHIS, TN (US)

(72) Inventor: Monica M. JABLONSKI, Memphis, TN (US)

(21) Appl. No.: 18/420,262

(22) Filed: Jan. 23, 2024

Related U.S. Application Data
(63) Continuation of application No. 18/022,252, filed on Feb. 20, 2023.
(60) Provisional application No. 63/068,917, filed on Aug. 21, 2020.

(51) Int. Cl. A61K 31/197; A61K 9/00; A61K 9/113; A61P 27/06

(52) U.S. Cl. CPC A61K 31/197; A61K 9/0048; A61K 9/113; A61P 27/06

(57) ABSTRACT

This invention is directed to a method of preventing ocular neurodegeneration in a subject in need thereof.

This application claims priority from U.S. Provisional Application No. 63/068,917, filed on Aug. 21, 2020, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

### GOVERNMENT INTERESTS

This invention was made with government support under Grant No. R43 EY029909 and R24 EY029950 awarded by the National Institutes of Health. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to a method of preventing ocular neurodegeneration in a subject in need thereof.

### BACKGROUND OF THE INVENTION

Glaucoma is the leading cause of irreversible blindness in the world. This disease now affects more than 3 million people in the United States, and with the projected increased in longevity, this number could increase to ˜6.3 million by 2050. There are four major types of adult-onset glaucoma, all of which lead to vision loss through a final pathway of retinal ganglion cell (RGC) dysfunction and/or death. Interestingly, each form of glaucoma can be associated with multiple and sometimes divergent risk factors, indicating there are multiple triggering mechanisms leading to RGC demise. For three of four adult-onset glaucoma subtypes, elevated intraocular pressure (IOP) is the most significant predictive risk factor for visual field loss subsequent to RGC demise. The fourth sub-type, normal tension glaucoma, is not associated with high IOP and factors that trigger RGC death are largely unknown.

The current standard of care for adult-onset glaucoma includes treatment with IOP-lowering medications delivered topically as eye drops. The major limitation of all currently FDA-approved glaucoma medications is limited efficacy. Specifically, IOP reduction does not fully prevent RGC death and resulting visual field loss in many glaucoma patients.

### SUMMARY OF THE INVENTION

An aspect of the invention is directed towards methods of treating glaucoma in a subject comprising administering to the subject in need thereof an effective amount of an ocular composition comprising pregabalin (PRG). In embodiments, the composition decreases intraocular pressure (IOP) of the eye. In embodiments, the ocular composition contains about 0.001% to about 1.2% of pregabalin (PRG). In embodiments, the neuroprotective effects affect retinal ganglion cells (RGC) and optic nerve. In embodiments, the composition is administered topically. In a further embodiment, the topically administered composition is administered to one or both eyes of the subject. In a further embodiment, the topically administered composition is administered via eye drops. In a further embodiment, the topically administered composition is administered once per day. In an embodiment, the composition comprises a microemulsion (ME) formulation. In a further embodiment, the microemulsion formulation results in increased duration of action and increased efficacy. In another embodiment, the microemulsion comprises a water-in-oil-in-water (W₁/O/W₂) multiple microemulsion. In a further embodiment, the multiple microemulsion comprises: a. an internal phase comprising an aqueous solution (W₁) encompassed within an internal emulsifier; b. an intermediate oil phase (O) encompassing the internal phase encompassed within an external emulsifier; and c. an external aqueous phase surrounding the external emulsifier (W₂). In a further embodiment, the intermediate oil phase (O) comprises an internal emulsifier. In a further embodiment, the internal emulsifier comprises caproyl 90, lecithin, or a combination thereof. In an embodiment, the aqueous solution is selected from the group consisting of deionized waster, saline, phosphate buffered saline, artificial tears, and a balanced salt solution. In an embodiment, the external aqueous phase (W₂) comprises the external emulsifier and bioadhesive polymers. In an embodiment, the ME is formulated as a topical formulation. In another embodiment, the multiple microemulsion further comprises an insoluble or sparing soluble drug in the oil phase (O). In an embodiment, multiple microemulsion further comprises a water-soluble drug in the aqueous solution (W₂). In embodiments, the ocular composition or microemulsion formulation is administered in a single dose. In embodiments, the ocular composition or microemulsion formulation is serially dosed. In embodiments, the ocular composition or microemulsion formulation is administered to a subject once daily, twice daily, trice daily, once every few days, or once weekly.

An aspect of the invention is directed towards methods of preventing (such as protecting against) glaucoma-induced neurodegeneration comprising administering to the subject in need thereof an effective amount of an ocular composition comprising pregabalin (PRG). In embodiments, the composition decreases intraocular pressure (IOP) of the eye. In embodiments, the ocular composition contains about 0.001% to about 1.2% of pregabalin (PRG). In embodiments, the neuroprotective effects affect retinal ganglion cells (RGC) and optic nerve. In embodiments, the composition is administered topically. In a further embodiment, the topically administered composition is administered to one or both eyes of the subject. In a further embodiment, the topically administered composition is administered via eye drops. In a further embodiment, the topically administered composition is administered once per day. In an embodiment, the composition comprises a microemulsion (ME) formulation. In a further embodiment, the microemulsion formulation results in increased duration of action and increased efficacy. In another embodiment, the microemulsion comprises a water-in-oil-in-water (W₁/O/W₂) multiple microemulsion. In a further embodiment, the multiple microemulsion comprises: a. an internal phase comprising an aqueous solution (W₁) encompassed within an internal emulsifier; b. an intermediate oil phase (O) encompassing the internal phase encompassed within an external emulsifier; and c. an external aqueous phase surrounding the external emulsifier (W₂). In a further embodiment, the intermediate oil phase (O) comprises an internal emulsifier. In a further embodiment, the internal emulsifier comprises caproyl 90, lecithin, or a combination thereof. In an embodiment, the aqueous solution is selected from the group consisting of deionized waster, saline, phosphate buffered saline, artificial tears, and a balanced salt solution. In an embodiment, the external aqueous phase (W₂) comprises the external emulsifier and bioadhesive polymers. In an embodiment, the ME is formulated as a topical formulation. In another embodiment, the multiple microemulsion further comprises an insoluble or sparing soluble drug in the oil phase (O). In an embodiment, multiple microemulsion further comprises a water-soluble drug in the aqueous solution (W₂). In embodiments, the ocular composition or microemulsion formulation is administered in a single dose. In embodiments, the ocular composition or microemulsion formulation is serially dosed. In embodiments, the ocular composition or microemulsion formulation is administered to a subject once daily, twice daily, trice daily, once every few days, or once weekly.

An aspect of the invention is directed towards methods of decreasing visual field loss in a subject comprising administering to the subject in need thereof an effective amount of an ocular composition comprising pregabalin (PRG). In embodiments, the composition decreases intraocular pressure (TOP) of the eye. In embodiments, the ocular composition contains about 0.001% to about 1.2% of pregabalin (PRG). In embodiments, the neuroprotective effects affect retinal ganglion cells (RGC) and optic nerve. In embodiments, the composition is administered topically. In a further embodiment, the topically administered composition is administered to one or both eyes of the subject. In a further embodiment, the topically administered composition is administered via eye drops. In a further embodiment, the topically administered composition is administered once per day. In an embodiment, the composition comprises a microemulsion (ME) formulation. In a further embodiment, the microemulsion formulation results in increased duration of action and increased efficacy. In another embodiment, the microemulsion comprises a water-in-oil-in-water (W₁/O/W₂) multiple microemulsion. In a further embodiment, the multiple microemulsion comprises: a. an internal phase comprising an aqueous solution (W₁) encompassed within an internal emulsifier; b. an intermediate oil phase (O) encompassing the internal phase encompassed within an external emulsifier; and c. an external aqueous phase surrounding the external emulsifier (W₂). In a further embodiment, the intermediate oil phase (O) comprises an internal emulsifier. In a further embodiment, the internal emulsifier comprises caproyl 90, lecithin, or a combination thereof. In an embodiment, the aqueous solution is selected from the group consisting of deionized waster, saline, phosphate buffered saline, artificial tears, and a balanced salt solution. In an embodiment, the external aqueous phase (W₂) comprises the external emulsifier and bioadhesive polymers. In an embodiment, the ME is formulated as a topical formulation. In another embodiment, the multiple microemulsion further comprises an insoluble or sparing soluble drug in the oil phase (O). In an embodiment, multiple microemulsion further comprises a water-soluble drug in the aqueous solution (W₂). In embodiments, the ocular composition or microemulsion formulation is administered in a single dose. In embodiments, the ocular composition or microemulsion formulation is serially dosed. In embodiments, the ocular composition or microemulsion formulation is administered to a subject once daily, twice daily, trice daily, once every few days, or once weekly.

An aspect of the invention is directed towards methods of decreasing intraocular pressure and preventing ocular neurodegeneration comprising administering to the subject in need thereof an effective amount of an ocular composition comprising pregabalin (PRG). In embodiments, the composition decreases intraocular pressure (IOP) of the eye. In embodiments, the ocular composition contains about 0.001% to about 1.2% of pregabalin (PRG). In embodiments, the neuroprotective effects affect retinal ganglion cells (RGC) and optic nerve. In embodiments, the composition is administered topically. In a further embodiment, the topically administered composition is administered to one or both eyes of the subject. In a further embodiment, the topically administered composition is administered via eye drops. In a further embodiment, the topically administered composition is administered once per day. In an embodiment, the composition comprises a microemulsion (ME) formulation. In a further embodiment, the microemulsion formulation results in increased duration of action and increased efficacy. In another embodiment, the microemulsion comprises a water-in-oil-in-water (W₁/O/W₂) multiple microemulsion. In a further embodiment, the multiple microemulsion comprises: a. an internal phase comprising an aqueous solution (W₁) encompassed within an internal emulsifier; b. an intermediate oil phase (O) encompassing the internal phase encompassed within an external emulsifier; and c. an external aqueous phase surrounding the external emulsifier (W₂). In a further embodiment, the intermediate oil phase (O) comprises an internal emulsifier. In a further embodiment, the internal emulsifier comprises caproyl 90, lecithin, or a combination thereof. In an embodiment, the aqueous solution is selected from the group consisting of deionized waster, saline, phosphate buffered saline, artificial tears, and a balanced salt solution. In an embodiment, the external aqueous phase (W₂) comprises the external emulsifier and bioadhesive polymers. In an embodiment, the MW is formulated as a topical formulation. In another embodiment, the multiple microemulsion further comprises an insoluble or sparing soluble drug in the oil phase (O). In an embodiment, multiple microemulsion further comprises a water-soluble drug in the aqueous solution (W₂). In embodiments, the ocular composition or microemulsion formulation is administered in a single dose. In embodiments, the ocular composition or microemulsion formulation is serially dosed. In embodiments, the ocular composition or microemulsion formulation is administered to a subject once daily, twice daily, trice daily, once every few days, or once weekly.

An aspect of the invention is directed towards methods for providing neuroprotective effects to the eye of a subject in need thereof comprising administering to the subject an effective amount of an ocular composition comprising pregabalin (PRG). In embodiments, the composition decreases intraocular pressure (IOP) of the eye. In embodiments, the ocular composition contains about 0.001% to about 1.2% of pregabalin (PRG). In embodiments, the neuroprotective effects affect retinal ganglion cells (RGC) and optic nerve. In embodiments, the composition is administered topically. In a further embodiment, the topically administered composition is administered to one or both eyes of the subject. In a further embodiment, the topically administered composition is administered via eye drops. In a further embodiment, the topically administered composition is administered once per day. In an embodiment, the composition comprises a microemulsion (ME) formulation. In a further embodiment, the microemulsion formulation results in increased duration of action and increased efficacy. In another embodiment, the microemulsion comprises a water-in-oil-in-water (W₁/O/W₂) multiple microemulsion. In a further embodiment, the multiple microemulsion comprises: a. an internal phase comprising an aqueous solution (W₁) encompassed within an internal emulsifier; b. an intermediate oil phase (O) encompassing the internal phase encompassed within an external emulsifier; and c. an external aqueous phase surrounding the external emulsifier (W₂). In a further embodiment, the intermediate oil phase (O) comprises an internal emulsifier. In a further embodiment, the internal emulsifier comprises caproyl 90, lecithin, or a combination thereof. In an embodiment, the aqueous solution is selected from the group consisting of deionized waster, saline, phosphate buffered saline, artificial tears, and a balanced salt solution. In an embodiment, the external aqueous phase (W₂) comprises the external emulsifier and bioadhesive polymers. In an embodiment, the ME is formulated as a topical formulation. In another embodiment, the multiple microemulsion further comprises an insoluble or sparing soluble drug in the oil phase (O). In an embodiment, multiple microemulsion further comprises a water-soluble drug in the aqueous solution (W₂). In embodiments, the ocular composition or microemulsion formulation is administered in a single dose. In embodiments, the ocular composition or microemulsion formulation is serially dosed. In embodiments, the ocular composition or microemulsion formulation is administered to a subject once daily, twice daily, trice daily, once every few days, or once weekly.

An aspect of the invention is directed towards methods of lowering intraocular eye pressure (IOP) and providing direct neuroprotection comprising administering a therapeutic targeted to the calcium channel, voltage-dependent α2δ1 subunit (CACNA2D1) protein encoded by the Cacna2dq gene.

Other objects and advantages of this invention will become readily apparent from the ensuing description.

### BRIEF DESCRIPTION OF THE FIGURES

FIG. 1 shows an IOP modulating QTL on proximal Chr 5. Cacna2d1 was identified as the IOP-modulating gene (Chintalapudi et al. Nature Communications 8, 1755 (2017)).

FIG. 2 shows that CACNA2D1 is localized to the ciliary body, Schlemm's canal and trabecular meshwork (Chintalapudi et al. Nature Communications 8, 1755 (2017)).

FIG. 3 shows that CACNA2D1 is localized to retinal ganglion cells (RGCs) (red arrows) and their axons in the nerve fiber layer (NFL) (white arrows) (Panels A and B). It is also in the RGC axon bundles in the optic nerve (ON) (black arrows) and the myelin sheath surrounding the nerve (white arrows) (Panel C).

FIG. 4 shows that shows that in BXD29 mice, IOP (circles) is relatively constant throughout its life, yet axons in the ON die early (2-5 mo).

FIG. 5 shows that the baseline IOP of B6 mice (15.83±0.73 mmHg) and BXD29 mice (16.25±0.25 mmHg) are not significantly different at 5 weeks of age. Unlike B6, the IOP of BXD29 mice is unresponsive to topical PRG-ME (n=5 mice/condition).

FIG. 6 shows PRG-ME (squares) greatly improves drug efficacy over PRG in a viscous medium (upward facing triangle) modified from (Ibrahim, M. et al. ACS Nano 13, 13728-13744 (2019)).

FIG. 7 shows once daily dosing for 21 days with PRG-ME (solid squares) keeps IOP in a lower, physiological range compared to blank ME. No drug tolerance is detected (Ibrahim, M. et al. ACS Nano 13, 13728-13744 (2019)).

FIG. 8 shows PRG reaches the retina (far right) after topical dosing with ME (21 days daily dosing) (Ibrahim, M. et al. ACS Nano 13, 13728-13744 (2019)).

FIG. 9 shows that topical daily dosing with PRG ME protects the RGC axons in the ON from degenerating. At 5 weeks of age (Panel A), the ON of BXD29 mice is healthy. After daily topical dosing with our PRG ME for 6 weeks (Panel B), the structure of the ON is well preserved and similar to pre-treatment controls. In marked contrast, ON axons from mice dosed with blank ME for 6 weeks (panel C) have degenerated.

FIG. 10 is a schematic that shows, without wishing to be bound by theory, binding of PRG (Pregabalin) to CACNA2D1 (red) causes the al pore of the calcium channel to restrict Ca²⁺ influx into RGCs and their axons.

FIG. 11 shows a healthy optic nerve in a Dutch belted rabbit treated with our pregabalin microemulsion for 60 days.

FIG. 12 shows the PRG ME has a distinct multi-layered structure as shows by transmission electron microscopy (TEM) (Ibrahim, M. et al. ACS Nano 13, 13728-13744 (2019)).

FIG. 13 shows PRG ME (square) prolongs PRG release for >24 hours (compared to eye drops (circle) or water (asterisk).

FIG. 14 shows a flow diagram illustrating ME synthesis.

FIG. 15 shows a cartoon of the ME with aqueous phases indicated by blue and the oil phase by tan.

FIG. 16 shows after the first dose of the PRG ME, IOP remained at a reduced level for 21 days of dosing in the Dutch belted rabbit. The eye receiving blank ME remained at the elevated baseline IOP (Ibrahim, M. et al. ACS Nano 13, 13728-13744 (2019)).

FIG. 17 shows Dutch belted rabbit eyes appear healthy after 21 days of dosing (Ibrahim, M. et al. ACS Nano 13, 13728-13744 (2019)).

FIG. 18 shows minimal cupping of the optic nerve head of a rabbit treated with our pregabalin microemulsion for 60 days.

FIG. 19 shows a fundus image with a cup-to-disc ratio of <0.4 in the eye of a Dutch belted rabbit treated with the pregabalin microemulsion for 60 days.

FIG. 20 shows an image of a naïve Dutch belted rabbit ocular nerve cross section with the damaged area circled.

FIG. 21 shows an image of cup-to-disc ratio of >0.7 in the eye of a naïve Dutch belted rabbit.

FIG. 22 shows pregabalin concentration in eye compartments from Dutch belted rabbits treated with our pregabalin microemulsion.

FIG. 23 shows a graph of cup-to-disc ratio between a treated eye and a naïve eye of Dutch belted rabbits.

FIG. 24 shows that topically applied aqueous pregabalin lowers intraocular pressure (IOP) by 20% but the IOP returns to baseline by 7 hours after dosing.

FIG. 25 shows PRG reaches the retina (green) after topical dosing with ME in the Dutch belted rabbit (21 days daily dosing) (Ibrahim, M. et al. ACS Nano 13, 13728-13744 (2019)).

FIG. 26 shows that minimal PRG reaches the fellow eye of the Dutch belted rabbit after topical dosing with ME (21 days daily dosing) (Ibrahim, M. et al. ACS Nano 13, 13728-13744 (2019)).

FIG. 27 shows after the first dose of the PRG ME to the eye of Dutch belted rabbits, IOP remained at a reduced level for 60 days. The fellow eye receiving blank ME remained at the elevated baseline IOP.

FIG. 28 shows Dutch belted rabbit eyes appear healthy after 60 days of dosing.

FIG. 29 shows a fundus image with a cup-to-disc ratio of <0.4 in the fellow eye of a Dutch belted rabbit that received only blank ME for 60 days.

FIG. 30 shows a graph of cup-to-disc ratio between a treated eye and a naïve eye of Dutch belted rabbits.

FIG. 31 shows a healthy optic nerve in the fellow eye of a Dutch belted rabbit treated with blank microemulsion for 60 days.

FIG. 32 shows minimal cupping of the optic nerve head of the fellow eye of a Dutch belted rabbit treated with blank microemulsion for 60 days.

FIG. 33 shows large and deep cupping of the optic nerve head of a naïve Dutch belted rabbit.

FIG. 34 shows pregabalin concentration in peripheral organs from Dutch belted rabbits treated for 60 days with the blank microemulsion in one eye and blank microemulsion in the fellow eye.

FIG. 35 shows that the IOP of BXD29 mice is not responsive to topical pregabalin microemulsion up to 6 weeks of daily dosing. The IOP of dosed and undosed eyes are statistically identical at all time points.

FIG. 36 shows a graph of the number of necrotic axons under conditions described in FIG. 35(n=6 mice/condition).

FIG. 37 shows confocal microscopy images. Calcium channel subunits CACNA2D1 and CACNA2D2 are expressed in the ciliary body (CB), trabecular meshwork retina and optic nerve head (ONH). Sections of whole C57Bl/6J mice at 1 month of age were immunolabeled for the two targets of pregabalin calcium voltage-gated channel auxiliary subunit alpha 2 delta 1 (CACNA2D1, red) and calcium voltage-gated channel auxiliary subunit alpha 2 delta 2 (CACNA2D2, green). Nuclei labeled with DAPI (blue). Panels A-C) Section through the ciliary body. Panels D-F) Retinal section through the optic nerve head. Scale bar=20 mm.

FIG. 38 shows an image in which immunohistochemistry is used to show localization of CACNA2D1 in the retina. The CACNA2D1 protein is found in the inner plexiform layer (IPL) and the photoreceptors.

FIG. 39 shows a graph of pregabalin concentrations. After topical dosing with our ME, pregabalin reaches the retina and ON.

FIG. 40 shows the OBI apparatus for producing blast injury to the eye of a DB rabbit.

FIG. 41 shows a graph of IOP measurements. IOP was unchanged from baseline up to 7 weeks post-injury. Data is from 50 psi blast pressure.

FIG. 42 shows a graph of pattern electroretinography (PERG). PERG amplitudes transiently increased followed by a rapid decrease after 2 weeks post-injury. Data is from 50 psi blast pressure.

FIG. 43 shows a graph of cup/disc ratio. By 2 weeks post-injury, the cup to disc ratio steadily increased, which can be indicative of RGC axon loss. Data is from 50 psi blast pressure.

FIG. 44 shows histology images. 9-month-old naïve DB rabbits with elevated IOP develop ON damage and ONH cupping.

FIG. 45 shows histology images. 9-month-old rabbits treated for 2 months with pregabalin ME do not develop ON damage of ONH cupping.

FIG. 46 shows microscopy images. Ca²⁺ indicators loaded into RGCs (panel A). Higher magnification of RGC axons (square in panel B as well as panel C) and soma (circle in panel B as well as panel E). K⁺-induced in an axon bundle (panel D) and an individual RGC (panel F). Ca²⁺ transients are shown in purple.

### DETAILED DESCRIPTION OF THE INVENTION

Aspects of the invention are drawn to methods of preventing ocular neurodegeneration to a subject in need thereof.

Detailed descriptions of one or more embodiments are provided herein. However, the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” can follow unless explicitly stated otherwise. Similarly, “an example,” “exemplary” and the like are nonlimiting.

The term “substantially” allows for deviations from the descriptor that do not negatively impact the intended purpose. Descriptive terms can be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Aspects of the invention are drawn towards a method of preventing (such as protecting against) ocular neurodegeneration comprising administering a composition comprising pregabalin (PRG) to a subject in need thereof. As used herein, “neurodegeneration” can refer to death/dysfunction of retinal ganglion cells (RGCs) and/or optic nerve. For example, neurodegeneration can be measured using visual field testing by one skilled in the art. For example, neurodegeneration can be quantified by measuring the function of the RGCs using the pattern electroretinogram (pERG) by one skilled in the art.

Aspects of the invention are drawn towards a method of providing neuroenhancement and/or neuroprotection. For example, embodiments can comprise administering a composition comprising pregabalin (PRG) to a subject in need thereof. As used herein, “neuroenhancement” can refer to an increase in the health or performance of a neurological component. Non-limiting examples of a “neurological component” can be retinal ganglion cells, or their respective axons and photoreceptors. As used herein, the term “neuroprotection” can refer to the prevention of neuronal cell death. For example, administration of a pregabalin-containing ME described herein can provide neuroenhancement and/or neuroprotective effects to a subject in need thereof.

Aspects of the invention are drawn towards a method of treating glaucoma or providing neuroprotective effects in a subject comprising administering an effective amount of an ocular composition comprising pregabalin (PRG). Aspects of the invention are drawn towards a method of decreasing visual field loss in a subject comprising administering to the subject in need thereof an effective amount of an ocular composition comprising PRG. Aspects of the invention are drawn towards a method of decreasing intraocular eye pressure and preventing against neurodegeneration comprising administering to the subject in need thereof an effective amount of an ocular composition comprising PRG. As used herein, the term “ocular composition” can refer to any composition or compound formulated for ocular administration or that which can be used to treat an ocular disease or disorder.

As used herein, an “effective amount” can refer to the dose or concentration of a drug that produces a biological response.

Aspects of the invention are drawn towards a method of providing neuroprotective effects to the eye of a subject in need thereof. For example, embodiments can comprise administering to the subject a composition comprising (PRG). As used herein, the phrase “neuroprotective effects” can refer to preventing and/or reducing death/dysfunction of retinal ganglion cells (RGCs) and/or optic nerve. For example, neuroprotective effects can be measured using visual field testing by one skilled in the art. For example, neuroprotective effects can be quantified by measuring the function of the RGCs using the pattern electroretinogram (pERG) by one skilled in the art. In embodiments, the composition decreases intraocular eye pressure (IOP) of the eye, while providing neuroprotective effects. In embodiments, the composition provides neuroprotective effects while having no effect of IOP. In embodiments, the neuroprotective effect is independent of any changes in IOP.

As used herein, the term “administering” can refer to introducing a substance into a subject. Any route of administration can be utilized including, for example, intranasal, topical, oral, parenteral, intravitreal, intraocular, ocular, subretinal, intrathecal, intravenous, subcutaneous, transcutaneous, intracutaneous, intracranial and the like administration. In embodiments, “administering” can also refer to providing a therapeutically effective amount of a formulation or pharmaceutical composition to a subject. The formulation or pharmaceutical compound of the present invention can be administered alone, but can be administered with other compounds, excipients, fillers, binders, carriers or other vehicles selected based upon the chosen route of administration and standard pharmaceutical practice. Administration can be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

Different forms of the formulation can be calibrated in order to adapt both to different individuals and to the different needs of a single individual. However, the formulation need not counter every cause in every individual. Rather, by countering the necessary causes, the formulation will restore the body and brain to their normal function. Then the body and brain themselves will correct the remaining deficiencies.

The term “therapeutically effective amount” can refer to that amount of an embodiment of the composition or pharmaceutical composition being administered that can relieve to some extent one or more of the symptoms of the disease or condition being treated, and/or that amount that can prevent, to some extent, one or more of the symptoms of the condition or disease that the subject being treated has or is at risk of developing.

As used interchangeably herein, “subject,” “individual,” or “patient,” can refer to a vertebrate, for example, a mammal, such as a human. Mammals can include, but are not limited to, murines, simians, humans, farm animals, sport animals, and pets. The term “pet” can include a dog, cat, guinea pig, mouse, rat, rabbit, ferret, and the like. The term farm animal can include a horse, sheep, goat, chicken, pig, cow, donkey, llama, alpaca, turkey, and the like.

A “pharmaceutically acceptable excipient,” “pharmaceutically acceptable diluent,” “pharmaceutically acceptable carrier,” or “pharmaceutically acceptable adjuvant” can refer to an excipient, diluent, carrier, and/or adjuvant that are useful in preparing a pharmaceutical composition that are safe, non-toxic and neither biologically nor otherwise undesirable, and can include an excipient, diluent, carrier, and adjuvant that are acceptable for veterinary use and/or human pharmaceutical use. A “pharmaceutically acceptable excipient, diluent, carrier and/or adjuvant” as used herein can include one and more such excipients, diluents, carriers, and adjuvants.

The phrase “pharmaceutical composition” or a “pharmaceutical formulation” can refer to a composition or pharmaceutical composition suitable for administration to a subject, such as a mammal, especially a human and that can refer to the combination of an active agent(s), or ingredient with a pharmaceutically acceptable carrier or excipient, making the composition suitable for diagnostic, therapeutic, or preventive use in vitro, in vivo, or ex vivo. In a “pharmaceutical composition” can refer to the composition being sterile, and free of contaminants that can elicit an undesirable response within the subject (e.g., the compound(s) in the pharmaceutical composition is pharmaceutical grade). Pharmaceutical compositions can be designed for administration to subjects or patients in need thereof via a number of different routes of administration including oral, intranasal, topical, intravenous, buccal, rectal, parenteral, intraperitoneal, intradermal, intracheal, intramuscular, subcutaneous, by stent-eluting devices, catheters-eluting devices, intravascular balloons, inhalational and the like.

In embodiments, the method comprises administering the composition topically. For example, the composition is administered via eye drops.

An aspect of the invention is drawn towards administering the composition more than 3 times a day, about 3 times a day, about twice a day, about once a day, about once every two days, about once every three days, about once every 4 days, about once every 5 days, about once every 6 days, about once every week, about once every 2 weeks, about once every 3 weeks, about once every month, about once every 2 months, about once every 3 months, about once every 4 months, about once every five months, about once every 6 months, about once every 7 months, about once every 8 month, about once every 9 months, about once every 10 months, about once every 11 months, about once a year, or less than once a year.

In an embodiment, the composition comprises a microemulsion. See, for example, US2020/0383915. As used herein, “microemulsion” can refer to a thermodynamically stable dispersion of one liquid phase into another stabilized by an interfacial film of surfactant. As used herein, “surfactant” can refer to a synthetic and/or naturally occurring amphiphilic molecules that have hydrophobic portion(s) and hydrophilic portion(s). For example, a surfactant can refer to an emulsifier. As used herein, “emulsifier” refers to a substance that stabilizes an emulsion and/or microemulsion. For example, an emulsifier can refer to a compound comprising one or more molecules, compounds, or ingredients for emulsifying or stabilizing a water-in-oil microemulsion (W/O) or an oil-in-water (O/W) microemulsion.

In an embodiment, the microemulsion is multilayer microemulsion. As used herein, the phrase “multiple microemulsion” can refer to a thermodynamically stable dispersion of one liquid known as a discontinuous (or intermediate phase), into another liquid know as a continuous phase (or external phase) in which the droplets of the discontinuous phase contain smaller droplets of the same nature of the continuous phase known as the internal phase. For example, the multiple microemulsion is a water-in-oil-in-water (W₁/O/W₂) microemulsion comprising: a discontinuous phase comprising an aqueous solution (W₁) encompassed within an internal emulsifier; a continuous phase oil phase (O) encompassing the internal phase encompassed within an external emulsifier; and a continuous aqueous phase surrounding the external emulsifier (W₂). As used herein “discontinuous phase” can refer to elements dispersed within, and immiscible with, a continuous phase and can be used interchangeably with “intermediate phase or oil phase”. As used herein, “continuous phase” can refer to the phase which with an immissible phase is dispersed and can be used interchangeable with “external phase”. As used herein, “internal phase” can refer to a phase of the same nature as external phase and dispersed as smaller droplets within the discontinuous phase. As used herein, “internal emulsifier” can refer to hydrophobic surfactant(s) with HLB value 3-7 that located at the interface between the internal and intermediate phases. As used herein, “external emulsifier” can refer to hydrophilic surfactant(s) with HLB value higher than 10 that located at the interface between the intermediate and external phases. Hydrophile Lipophile Balance (HLB) can refer to the degree of affinity of a surfactant to water or oil.

In an embodiment, the discontinuous oil phase (O) comprises an internal emulsifier. For example, the internal emulsifier comprises caproyl 90, lecithin, or a combination thereof. In an embodiment, the discontinuous oil phase (O) comprises an insoluble or sparingly soluble drug.

In an embodiment, the aqueous solution (W₂) comprises a water-soluble drug.

In an embodiment the aqueous solution is selected from the group consisting of deionized water, saline, phosphate buffered saline, artificial tears, and a balanced salt solution.

In an embodiment, the continuous aqueous phase (W₂) comprises an external emulsifier and bioadhesive polymers. In an embodiment, a bioadhesive polymer can refer to a mucoadhesive polymer.

In an embodiment, the microemulsion formulation results in increased duration of action and increased efficacy. As used herein, the term “duration of action” can refer to the length of time a substance is effective. For example, putting PRG in the microemulsion prolongs the IOP-lowering and neuroprotective effects of PRG. For example, without the microemulsion, IOP is lowered by about 20% and returns to baseline by about 8-10 hours and with the microemulsion IOP is lowered by about 40% and doesn't return to baseline until about >30 hours after dosing.

Any suitable hydrophobic internal emulsifier can be used in the microemulsions. In various non-limiting embodiments, the internal emulsifier is selected from the group consisting of propylene glycol monocaprylate or any other surfactant with an Hydrophile-Lipophile Balance (HLB) value 3-7 and/or propylene glycol ester of any fatty acid such as; propylene glycol monocaproate, propylene glycol monocaprylate, propylene glycol monocaprate, propylene glycol monolaurate, propylene glycol monostearate, propylene glycol monopalmitate, polyethylene glycol lauryl ether, polyethylene glycol oleyl ether, polyethylene glycol hexadecyl ether, sorbitan monopalmitate, sorbitan monostearate, sorbitan monooleate, sorbitan monolaurate, transcutol P, gelucire 50/13 (mixture of PEG (MW 1500) mono-, di-, tri-esters of stearic acid), gelucire 44/14 (mixture of PEG (MW 1500) mono-, di-, tri-esters of lauric acid), gelucire 43/01 (mixture of PEG (MW 1500) mono-, di-, tri-esters of fatty acids C₈-C₁₈), any PEG mono-, di- and/or tri-esters of any fatty acid, lecithin, egg lecithin, phosphatidylcholine, phosphatidylethanolamine, phosphatidylinositol, tocopherol or any other phospholipid, and combinations thereof.

Capryol 90 is a surfactant with HLB=5. Its chemical name is propylene glycol monocaprylate (propylene glycol monoester of caprylic acid). Alternatives to propylene glycol monocaprylate may be any other surfactant with an HLB value 3-7 and/or propylene glycol ester of any fatty acid such as propylene glycol monocaproate, propylene glycol monocaprylate, propylene glycol monocaprate, propylene glycol monolaurate, propylene glycol monostearate, propylene glycol monopalmitate, polyethylene glycol lauryl ether, polyethylene glycol oleyl ether, polyethylene glycol hexadecyl ether, sorbitan monopalmitate, sorbitan monostearate, sorbitan monooleate, sorbitan monolaurate, etc.

Labrasol is a hydrophilic surfactant with HLB value=12. It consists of a small fraction of mono-, di- and triglycerides and mainly polyethylene glycol-8 (MW 400) mono- and diesters of caprylic and capric acids. Its chemical name is caprylocaproyl polyoxyl-8 glycerides, caprylocaproyl macrogol-8 glycerides or PEG-8 caprylic/capric glycerides. Its alternatives may be any other hydrophilic surfactant with HLB value (10-14) and/or polyethylene glycol mono- and/or di-esters of any fatty acid.

Cremophor EL is a hydrophilic surfactant with HLB value=14. Its chemical names are macrogolglycerol ricinoleate, PEG-35 castor oil, Polyoxyl 35 hydrogenated castor oil, or Polyoxyl-35 castor oil. Alternatives may be any other hydrophilic surfactant with HLB value (12-16) and/or polyethylene glycol mono- and/or di-esters of any fatty acid or fatty acid mixture.

Lecithin is a hydrophobic surfactant with HLB value=4-7. Its chemical name is 2-nonanoyloxy-3-octadeca-9,12-dienoyloxypropoxy)-[2-(trimethylazaniumyl)ethyl]phosphinate. It is a mixture of natural phospholipids so its alternatives may be one of the following: egg lecithin, phosphatidylcholine, phosphatidylethanolamine, phosphatidylinositol, tocopherol or any other phospholipid.

In one embodiment, the internal emulsifier is selected from the group consisting of propylene glycol monocaprylate, lecithin, and combinations thereof.

Any suitable aqueous solution can be used in the microemulsions. In various non-limiting embodiments, the aqueous solution is selected from the group consisting of deionized water, saline, phosphate buffered saline, artificial tears, and balanced salt solution.

Any suitable oil phase can be used in the microemulsions. In various non-limiting embodiments, the oil phase is selected from the group consisting of an oil that consists of medium chain triglycerides of caprylic (C₈) and capric (C₁₀) acids, any pure fatty acid ester including but not limited to ethyl, propyl, isopropyl, and butyl; esters of fatty acids including but not limited to caproic, caprylic, capric, lauric, palmitic, myristic, or stearic acids, isopropyl myristate, isopropyl palmitate, isopropyl caproate, isopropyl caprylate, ethyl stearate, butyl laurate, and any natural oil including but not limited to coconut oil, palm kernel oil, soya bean oil, castor oil, cotton seed oil, corn oil, and olive oil; and combinations thereof. In one specific embodiment, the oil phase comprises labrafac lipophile WL1349 (i.e., triglyceride esters of caprylic and capric acids).

Any suitable external emulsifier can be used in the MEs. In various other embodiments, the external emulsifier is selected from the group consisting of caprylocaproyl polyoxyl-8 glycerides, macrogolglycerol ricinoleate, any other hydrophilic surfactant with Hydrophile-Lipophile Balance (HLB) value between 10-16, polyethylene glycol mono- and/or di-esters of any fatty acid or fatty acid mixture, propylene glycol or any other alcohol including but not limited to glycerol, polyethylene glycol, ethanol, propanol, and isopropanol; and combinations thereof. In various further embodiments, the external emulsifier comprises caprylocaproyl polyoxyl-8 glycerides, macrogolglycerol ricinoleate, propylene glycol, or combinations thereof.

Any suitable combinations of the various components of the MEs of the disclosure may be used. In one embodiment, the ME contains 0.5-35% w/w aqueous solution, 0.5-95% w/w oil phase, and 5-99% w/w emulsifier (i.e.: internal emulsifier+external emulsifier). In another embodiment, the ME contains 10-30% w/w aqueous solution, 20-40% w/w oil phase, and 40-60% w/w emulsifier. In a further embodiment, the ME contains about 20% w/w aqueous solution, about 30% w/w oil phase, and about 50% w/w emulsifier. In various further embodiments, the ME contains at least 0.5% w/w aqueous solution, contains at least 1% w/w aqueous solution, contains at least 2% w/w aqueous solution, contains at least 3% w/w aqueous solution, contains at least 4% w/w aqueous solution, contains at least 5% w/w aqueous solution, contains at least 6% w/w aqueous solution, contains at least 7% w/w aqueous solution, contains at least 8% w/w aqueous solution, contains at least 9% w/w aqueous solution, contains at least 10% w/w aqueous solution, contains at least 11% w/w aqueous solution, contains at least 12% w/w aqueous solution, contains at least 13% w/w aqueous solution, contains at least 14% w/w aqueous solution, contains at least 15% w/w aqueous solution, contains at least 16% w/w aqueous solution, contains at least 17% w/w aqueous solution, contains at least 18% w/w aqueous solution, contains at least 19% w/w aqueous solution, contains at least 20% w/w aqueous solution, contains at least 25% w/w aqueous solution, contains at least 30% w/w aqueous solution, contains at least 35% w/w aqueous solution, contains at least 40% w/w aqueous solution, contains at least 45% w/w aqueous solution, contains at least 50% w/w aqueous solution, contains at least 55% w/w aqueous solution, contains at least 60% w/w aqueous solution, contains at least 65% w/w aqueous solution, contains at least 70% w/w aqueous solution, contains at least 75% w/w aqueous solution, contains at least 80% w/w aqueous solution, contains at least 85% w/w aqueous solution, contains at least 90% w/w aqueous solution, or contains at least 95% w/w aqueous solution.

In various embodiments, the external emulsifier is present in a ratio between about 10:1 and about 2:1 relative to the internal emulsifier. In various further embodiments, the external emulsifier is present in a ratio between about 9:1 and about 2:1, between about 8:1 and about 2:1, between about 7:1 and about 2:1, between about 6:1 and about 2:1, between about 5:1 and about 2:1, between about 4:1 and about 2:1, between about 3:1 and about 2:1, between about 10:1 and about 2.5:1, between about 9:1 and about 2.5:1, between about 8:1 and about 2.5:1, between about 7:1 and about 2.5:1, between about 6:1 and about 2.5:1, between about 5:1 and about 2.5:1, between about 4:1 and about 2.5:1, between about 3:1 and about 2.5:1, between about 10:1 and about 3:1 relative to the internal emulsifier, between about 9:1 and about 3:1, between about 8:1 and about 3:1, between about 7:1 and about 3:1, between about 6:1 and about 3:1, between about 5:1 and about 3:1, between about 4:1 and about 3:1, between about 10:1 and about 4:1 relative to the internal emulsifier, between about 9:1 and about 4:1, between about 8:1 and about 4:1, between about 7:1 and about 4:1, between about 6:1 and about 4:1, between about 5:1 and about 4:1, between about 10:1 and about 5:1 relative to the internal emulsifier, between about 9:1 and about 5:1, between about 8:1 and about 5:1, between about 7:1 and about 5:1, between about 6:1 and about 5:1, relative to the internal emulsifier.

In one embodiment, the aqueous solution comprises a water soluble therapeutic/drug. Any suitable water soluble therapeutic/drug may be incorporated in the aqueous solution, including but not limited to beta-blockers such as betaxolol and timolol; prostaglandin analogs such as bimatoprost, latanoprost, and travoprost; Alpha-adrenergic agents such as brimonidine tartrate; carbonic anhydrase inhibitors such as brinzolamide, dorzolamide, and acetazolamide; calcium channel blockers such as nimodipine and pregabalin; asialo, galactosylated, triantennary (NA3) (also known as asialo-, tri-antennary complex-type N-glycan), OT-551 hydrochloride (1-hydroxy-2,2,6,6-tetramethyl-4-piperidinyl cyclopropane carboxylic acid ester hydrochloride), brimonidine tartrate, clindamycin, ciprofloxacin, levofloxacin, gatifloxacin, gemifloxacin, ofloxacin, triamcinolone, valacyclovir, pyrimethamine, valganciclovir, ganciclovir, acyclovir, foscarnet, prednisolone acetate, diflupednate, triamcinolone, dexamethasone, methotrexate, azathioprine, mycophenolate mofetil, cyclosporine, tacrolimus, cyclophosphamide, ribavirin, bromfenac, ketorolac, nepafenac, lifitegrast, flubiprofen, diclonfenac, ketotifen, nedocromil, phenylephrine, azelastine, epinastine, naphazoline/pheniramine, oloptadine, bepotastine, alacaftadine, pemirolast, tetrahydrozoline with or without zinc sulfate, Iodoxamide, naphazoline, phenylephrine, cromolyn, emedastine, oxymetazoline, xylometazoline, loratidine, desloratidine, phenylglycine, gabapentin, or combinations thereof. In specific embodiments, the water-soluble drug is selected from the group consisting of phenylglycine, gabapentin, pregabalin and ribavirin, or a pharmaceutically acceptable salt thereof.

Pharmaceutically acceptable salts can include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl) aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

In another embodiment, the aqueous phase comprises a hydrogel (i.e.: a gel or swollen network structured polymer matrix in which the liquid component is water or aqueous solution, emulsion or suspension). In one embodiment the hydrogel comprises bioadhesive polymers. In one embodiment, the bioadhesive polymers comprise mucoadhesive polymers. In one embodiment, the hydrogel comprises mucoadhesive polymers. Any suitable mucoadhesive polymers may be used, including but not limited to polyacrylic acid derivatives (including but not limited to CARBOPOL®, such as CARBOPOL® 981), alginic acid and its salts or derivatives (including but not limited to sodium alginate), chitosan and its derivatives, dextran and its derivatives, pectin and its derivatives, gelatin and its derivatives, polyvinylpyrrolidone and its derivatives, N-methylpyrrolidone and its derivatives, hyaluronic acid salts and derivatives thereof, gellan gum and derivatives thereof, xanthan gum and derivatives thereof, agar and derivatives thereof, glycocholic acid and its salts or derivatives, or combinations thereof. In specific embodiments, the mucoadhesive polymers are selected from the group consisting of polyacrylic acid derivatives (including but not limited to CARBOPOL©, such as CARBOPOL© 981), alginic acid and its salts or derivatives (including but not limited to sodium alginate), chitosan and its derivatives, or combinations thereof.

The microemulsion can be formulated for any suitable route of administration (i.e.: orally, topically, intranasally, parenterally, etc.), in dosage unit formulations of water soluble therapeutic loaded in the microemulsion. The formulation can include any other components suitable for an administrative route, including but not limited to conventional pharmaceutically acceptable carriers, adjuvants, and vehicles. In one embodiment, the microemulsion is formulated as a topical formulation, such as for delivery to the eye. For example, the topical formulation comprises eye drops. In an embodiment, the microemulsion is formulated for injectable administration.

“Formulation” can refer to any collection of components of a compound, mixture, or solution selected to provide optimal properties for a specified end use, including product specifications and/or service conditions. The term formulation can include liquids, semi-liquids, colloidal solutions, dispersions, emulsions, microemulsions, and nanoemulsions, including oil-in-water emulsions and water-in-oil emulsions, pastes, powders, and suspensions. The formulations can also be can included, or packaged, with other non-toxic compounds, such as cosmetic carriers, excipients, binders and fillers, and the like. For example, the acceptable cosmetic carriers, excipients, binders, and fillers can include those which render the compounds amenable to oral delivery and/or provide stability such that the formulations of the present invention exhibit a commercially acceptable storage shelf life.

The microemulsions disclosed herein can be provided as microemulsion globules (i.e., drops). microemulsion globules can be of any suitable size. In one embodiment, the microemulsion globule is between about 1 nm and about 200 nm in diameter. In various further embodiments, ME globules are between about 1 nm and about 150 nm, about 1 nm and about 100 nm, about 1 nm and about 50 nm, about 1 nm and about 20 nm, about 1 nm and about 18 nm, about 1 nm and about 17 nm, about 5 nm and about 200 nm, about 5 nm and about 150 nm, about 5 nm and about 100 nm, about 5 nm and about 50 nm, about 5 nm and about 20 nm, about 5 nm and about 18 nm, about 5 nm and about 17 nm in diameter. In various further embodiments, the ME globules are about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 90 nm, about 95 nm, about 100 nm, about 110 nm, about 120 nm, about 130 nm, about 140 nm, about 150 nm, about 160 nm, about 170 nm, about 180 nm, about 190 or about 200 nm in diameter. In other embodiments, the ME globules are at least about 1 nm, at least 2 nm, at least 3 nm, at least 4 nm, at least 5 nm, at least 6 nm, at least 7 nm, at least 8 nm, at least 9 nm, at least 10 nm, at least 15 nm, at least 20 nm, at least 25 nm, at least 30 nm, at least 35 nm, at least 40 nm, at least 45 nm, at least 50 nm, at least 55 nm, at least 60 nm, at least 65 nm, at least 70 nm, at least 75 nm, at least 80 nm, at least 90 nm, at least 95 nm, at least 100 nm, at least 110 nm, at least 120 nm, at least 130 nm, at least 140 nm, at least 150 nm, at least 160 nm, at least 170 nm, at least 180 nm, at least 190 or at least 200 nm in diameter.

In another aspect is provided methods for treating an eye disease or providing neuroprotective effects. For example, embodiments can comprise administering to a subject in need thereof an amount effective to treat the eye disease or provide neuroprotective effects of the ME of any embodiment or combination of embodiments described herein, wherein the aqueous solution comprises a water-soluble therapeutic capable of treating the eye disease or providing neuroprotective effects. In various embodiments, the methods are for reducing intraocular pressure (TOP), treating glaucoma, preventing glaucoma-induced neurodegeneration, decreasing visual field loss, preventing neurodegeneration, and providing neuroprotective effects, comprising administering to a subject with elevated intraocular pressure, glaucoma, glaucoma-induced neurodegeneration, visual field loss, and/or neurodegeneration of the ME of any embodiment or combination of embodiments described herein, wherein the aqueous solution comprises a water soluble therapeutic capable of reducing IOP, treating glaucoma, preventing glaucoma-induced neurodegeneration, decreasing visual field loss, preventing neurodegeneration, and providing neuroprotective effects. In various embodiments, the water soluble therapeutic capable of reducing IOP, treating age-related macular degeneration, treating glaucoma, preventing glaucoma-induced neurodegeneration, decreasing visual field loss, preventing neurodegeneration, and providing neuroprotective effects is selected from the group consisting of beta-blockers such as betaxolol and timolol; prostaglandin analogs such as bimatoprost, latanoprost, and travoprost; Alpha-adrenergic agents such as brimonidine tartrate; carbonic anhydrase inhibitors such as brinzolamide, dorzolamide, and acetazolamide; calcium channel blockers such as nimodipine and pregabalin; asialo, galactosylated, triantennary (NA3) (also known as asialo-, tri-antennary complex-type N-glycan), OT-551 hydrochloride (1-hydroxy-2,2,6,6-tetramethyl-4-piperidinyl cyclopropane carboxylic acid ester hydrochloride), brimonidine tartrate, clindamycin, ciprofloxacin, levofloxacin, gatifloxacin, gemifloxacin, ofloxacin, triamcinolone, valacyclovir, pyrimethamine, valganciclovir, ganciclovir, acyclovir, foscarnet, prednisolone acetate, diflupednate, triamcinolone, dexamethasone, methotrexate, azathioprine, mycophenolate mofetil, cyclosporine, tacrolimus, cyclophosphamide, ribavirin, bromfenac, ketorolac, nepafenac, lifitegrast, flubiprofen, diclonfenac, ketotifen, nedocromil, phenylephrine, azelastine, epinastine, naphazoline/pheniramine, oloptadine, bepotastine, alacaftadine, pemirolast, tetrahydrozoline with or without zinc sulfate, Iodoxamide, naphazoline, phenylephrine, cromolyn, emedastine, oxymetazoline, xylometazoline, loratidine, desloratidine, phenylglycine, gabapentin, or combinations thereof. In specific embodiments, the water-soluble drug capable of reducing IOP, treating glaucoma, preventing glaucoma-induced neurodegeneration, decreasing visual field loss, preventing neurodegeneration, and providing neuroprotective effects is selected from the group consisting of phenylglycine, gabapentin, pregabalin and ribavirin, or a pharmaceutically acceptable salt thereof. In one specific embodiment, the water soluble drug capable of reducing TOP, treating glaucoma, preventing glaucoma-induced neurodegeneration, decreasing visual field loss, preventing neurodegeneration, and providing neuroprotective effects is pregabalin, and the pregabalin is present in the microemulsion at between about 0.2% to about 2% of the ME % w/w; in various further embodiments, the pregabalin is present in the ME at between about 0.2% to about 1.5%, between about 0.2% to about 1%, between about 2% to about 0.75%, between about 0.3% to about 2%, between about 0.3% to about 1.5%, between about 0.3% to about 1%, between about 0.3% to about 0.75%, between about 0.4% to about 2%, between about 0.4% to about 1.5%, between about 0.4% to about 1%, between about 0.4% to about 0.75%, between about 0.5% to about 2%, between about 0.5% to about 1.5%, between about 0.5% to about 1%, between about 0.5% to about 0.75%, between about 0.2% to about 0.6%, between about 0.3% to about 0.6%, between about 0.4% to about 0.6%, between about 0.5% to about 0.6%, about 0.2%, about 0.3%, about 0.4%, about 0.5%, or about 0.6% of the ME % w/w. The MEs described herein are excellent drug delivery systems for any water-soluble therapeutic candidate.

As used herein, “treatment” and “treating” can refer to the management and care of a subject for the purpose of combating a condition, disease or disorder, in any manner in which one or more of the symptoms of a disease or disorder are ameliorated or otherwise beneficially altered. The term can include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and can includes the administration of the active compounds to prevent or reduce the risk of the onset of symptoms or complications. Skilled artisans will appreciate a variety of methodologies and assays can be used to assess the development of pathology, and similarly, a variety of methodologies and assays can be used to reduce pathology, driveway, or regression. For example, methods described herein can provide for treatment against neurodegeneration or damage.

As used herein, the term “preventing” can refer to preventing a disease, disorder, or condition from occurring in a subject that may be at risk for the disease, but is not yet diagnosed as having the disease. Prevention (and effective dose to prevent) can be demonstrated in population studies. For example, an amount effective to prevent a given disease or condition is an amount effective to reduce the incidence in the treated population, compared to an untreated control population.

In other aspects of this embodiment, a water-soluble drug disclosed herein is used to reduce IOP and/or treat glaucoma (including POAG), protect against glaucoma-induced neurodegeneration, decrease visual field loss, protect against neurodegeneration, and provide neuroprotective effects in a patient suffering from one or more of these syndromes by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a water-soluble drug disclosed herein reduces IOP and/or treats glaucoma, protects against glaucoma-induced neurodegeneration, protects against glaucoma-induced neurodegeneration, decreases visual field loss, protects against neurodegeneration, and provides neuroprotective effects in a patient suffering from one of these syndromes from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

An microemulsion disclosed herein can comprise a water-soluble drug in an amount sufficient to allow customary administration to an individual. In aspects of this embodiment, a ME disclosed herein may include, e.g. at least 0.001% w/w, at least 0.002% w/w, at least 0.003% w/w, at least 0.004% w/w, at least 0.005% w/w, at least 0.006% w/w, at least 0.007% w/w, at least 0.008% w/w, at least 0.009% w/w, at least 0.01% w/w, at least 0.02% w/w, at least 0.03% w/w, at least 0.04% w/w, at least 0.05% w/w, at least 0.06% w/w, at least 0.07% w/w, at least 0.08% w/w, at least 0.09% w/w, at least 0.1% w/w, at least 0.2% w/w, at least 0.3% w/w, at least 0.4% w/w, 0.5% w/w, at least 0.6% w/w, at least 0.7% w/w, at least 0.8% w/w, at least 0.9% w/w, at least 1.0% w/w, at least 1.1% w/w, at least 1.2% w/w of a water-soluble drug. In yet other aspects of this embodiment, a ME disclosed herein may include, e.g., about 0.001% w/w to about 1.2% w/w, about 0.001% w/w to about 1.1% w/w, about 0.001% w/w to about 1.0% w/w, about 0.001% to about 0.9% w/w, about 0.001% w/w to about 0.8% w/w, about 0.3% w/w to about 1.2% w/w, about 0.3% w/w to about 1.10% w/w, about 0.3% w/w to about 1.0% w/w, about 0.3% w/w to about 0.9% w/w, about 0.3% w/w to about 0.8% w/w, about 0.4% w/w to about 1% w/w, about 0.4% w/w to about 0.9% w/w, about 0.4% w/w to about 0.8% w/w, about 0.4% w/w to about 0.7% w/w, about 0.5% w/w to about 1.0% w/w, about 0.5% w/w to about 0.9% w/w, about 0.5% w/w to about 0.8% w/w, about 0.5% w/w to about 0.7% w/w, about 0.55% w/w to about 0.8% w/w, or about 0.55% w/w to about 0.7% w/w of a water-soluble drug.

The final concentration of a water-soluble drug disclosed herein in a ME disclosed herein can be of any concentration desired. In an aspect of this embodiment, the final concentration of a water-soluble drug in a microemulsion can be a therapeutically effective amount. In other aspects of this embodiment, the final concentration of a water-soluble drug in a ME may be, e.g., at least 0.001% w/w, at least 0.002% w/w, at least 0.003% w/w, at least 0.004% w/w, at least 0.005% w/w, at least 0.006% w/w, at least 0.007% w/w, at least 0.008% w/w, at least 0.009% w/w, at least 0.01% w/w, at least 0.02% w/w, at least 0.03% w/w, at least 0.04% w/w, at least 0.05% w/w, at least 0.06% w/w, at least 0.07% w/w, at least 0.08% w/w, at least 0.09% w/w, at least 0.1% w/w, at least 0.2% w/w, at least 0.3% w/w, at least 0.4% w/w, 0.5% w/w, at least 0.6% w/w, at least 0.7% w/w, at least 0.8% w/w, at least 0.9% w/w, at least 1.0% w/w, at least 1.1% w/w, at least 1.2% w/w. In other aspects of this embodiment, the concentration of a water-soluble drug disclosed herein in a ME may be, e.g., at most 0.3% w/w, at most 0.4% w/w, at most 0.5% w/w, at most 0.6% w/w, at most 0.7% w/w, at most 0.8% w/w, at most 0.9% w/w, at most 1.0% w/w, at most 1.1% w/w, or at most 1.2% w/w. In other aspects of this embodiment, the final concentration of a water-soluble drug in a ME may be in a range of, e.g., about 0.001% w/w to about 1.2% w/w, about 0.001% w/w to about 1.1% w/w, about 0.001% w/w to about 1.0% w/w, about 0.001% to about 0.9% w/w, about 0.001% w/w to about 0.8% w/w, about 0.3% w/w to about 1.2% w/w, about 0.3% w/w to about 1.1% w/w, about 0.3% w/w to about 1.0% w/w, about 0.3% w/w to about 0.9% w/w, about 0.3% w/w to about 0.8% w/w, about 0.4% w/w to about 1% w/w, about 0.4% w/w to about 0.9% w/w, about 0.4% w/w to about 0.8% w/w, about 0.4% w/w to about 0.7% w/w, about 0.5% w/w to about 1.0% w/w, about 0.5% w/w to about 0.9% w/w, about 0.5% w/w to about 0.8% w/w, about 0.5% w/w to about 0.7% w/w, about 0.55% w/w to about 0.8% w/w, or about 0.55% w/w to about 0.7% w/w.

As used herein, “treat” or “treating” can refer to accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s). Certain embodiments disclose, in part, treating an individual suffering from IOP, glaucoma, glaucoma-induced neurodegeneration, decreased visual field loss, and/or neurodegeneration. In these embodiments, treating may refer to reducing or eliminating in an individual a clinical symptom of IOP, glaucoma, glaucoma-induced neurodegeneration, decreased visual field loss, and/or neurodegeneration; or delaying or preventing in an individual the onset of a clinical symptom of IOP, glaucoma, glaucoma-induced neurodegeneration, decreased visual field loss, and/or neurodegeneration. For example, the term “treating” can refer to reducing a symptom of a condition characterized by a IOP, glaucoma, glaucoma-induced neurodegeneration, decreased visual field loss, and/or neurodegeneration, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with IOP, glaucoma, age-related macular degeneration (AMD), uveitis, and/or conjunctivitis are well known and can be determined by a person of ordinary skill in the art by taking into account various factors associated with each of these syndromes. Those of skill in the art will know the appropriate symptoms or indicators associated with TOP, glaucoma, glaucoma-induced neurodegeneration, decreased visual field loss, and/or neurodegeneration and will know how to determine if an individual is a candidate for treatment as disclosed herein.

In various embodiments, a therapeutically effective amount of a water-soluble drug disclosed herein reduces a symptom associated with IOP, glaucoma, glaucoma-induced neurodegeneration, decreased visual field loss, and/or neurodegeneration by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other embodiments, a therapeutically effective amount of a water-soluble drug disclosed herein reduces a symptom associated with IOP, glaucoma, glaucoma-induced neurodegeneration, decreased visual field loss, and/or neurodegeneration by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other embodiments, a therapeutically effective amount of a water-soluble drug disclosed herein reduces a symptom associated with IOP, glaucoma, glaucoma-induced neurodegeneration, decreased visual field loss, and/or neurodegeneration by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

The therapeutics (such as the microemulsions) for use in the methods disclosed herein can be administered as deemed appropriate by attending medical personnel, for example, such as by routes of administration as described herein. In one embodiment, the therapeutics for use in the methods disclosed herein (such as the microemulsion) are administered to one or both eyes of the subject. In another embodiment, the administering is done once per day. Dosing can be single dosage or cumulative (serial dosing), and can be readily determined by one skilled in the art. For instance, treatment of IOP, glaucoma, glaucoma-induced neurodegeneration, decreased visual field loss, and/or neurodegeneration as well as providing neuroprotective effects may comprise a one-time administration of an effective dose of a ME containing a water-soluble drug disclosed herein. In another embodiment, treatment of IOP, glaucoma, glaucoma-induced neurodegeneration, decreased visual field loss, and/or neurodegeneration as well as providing neuroprotective effects can comprise multiple administrations of an effective dose of a microemulsion containing a water-soluble drug carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a ME containing a water-soluble drug disclosed herein can be administered to an individual once daily for an indefinite period of time, or until the individual no longer requires therapy. A person of ordinary skill in the art will recognize that the condition of the individual can be monitored throughout the course of treatment and that the effective amount of a ME containing a water-soluble drug disclosed herein that is administered can be adjusted accordingly.

In a further embodiment, a water-soluble drug of the invention and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a therapeutic for the treatment of IOP, glaucoma, glaucoma-induced neurodegeneration, decreased visual field loss, and/or neurodegeneration as well as providing neuroprotective effects is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped between period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In various embodiments, a therapeutically effective amount of a ME containing a water-soluble drug disclosed herein reduces intraocular pressure (IOP) within the eye of an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a ME containing a water-soluble drug disclosed herein reduces internal pressure within the eye in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a ME containing a water-soluble drug disclosed herein reduces internal pressure within the eye in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In embodiments, a therapeutically effective amount of a microemulsion containing a water-soluble drug disclosed herein is administered in a low dose, a mid-dose, or a high dose. As used herein, a “dose” can refer to an amount of a compound or composition that is effective to produce a biological response, such as an amount of pregabalin or a microemulsion comprising pregabalin that is effect to produce a biological response. For example, a dose can refer to a composition comprising about 0.1% of a compound, about 0.2% of a compound, about 0.3% of a compound, about 0.4% of a compound, about 0.5% of a compound, about 0.6% of a compound, about 0.7% of a compound, about 0.8% of a compound, about 0.9% of a compound, about 1% of a compound, about 2% of a compound, about 3% of a compound, about 4% of a compound, about 5% of a compound, about 10% of a compound, or greater than 10% of a compound. In embodiments, the dose can be about 0.6% pregabalin. For example, the dose can be a microemulsion comprising about 0.6% pregabalin. A “mid dose” can refer to, for example, about 3 times a low dose. A high dose can refer to, for example, about 10 times a low dose, or greater than about 10 times a low dose.

As used herein, the term “subject”, “individual,” or “patient,” used interchangeably, can refer to any animal, including mammals, such as mice, rats, other rodents, rabbits, dogs, cats, birds, swine, horses, livestock (e.g., pigs, sheep, goats, cattle), primates or humans. In specific embodiments, the subject, individual, or patient is a human. A pharmaceutical composition that includes a microemulsion and a water-soluble drug is administered to a subject. For example, any subject who is a candidate for treatment is a candidate with some form of IOP, glaucoma, age-related macular degeneration (AMD), uveitis, eye injury, inherited retinal degenerations, and/or conjunctivitis. Pre-operative evaluation includes routine history and physical examination in addition to thorough informed consent disclosing all relevant risks and benefits of the procedure.

The therapeutic-containing microemulsions for use in the methods disclosed herein can be formulated for and administered via any suitable route, including but not limited to oral, intravenous, ocular, intravaginal, intra-anal, subcutaneous, intracranial, topical, intramuscular, enteral or parenteral routes of administration. In embodiments, the therapeutic-containing MEs can be formulated for and administered via topical administration for ocular or optic application (including but not limited to being formulated as eye drops), intranasal administration, orally for different systemic diseases, transdermal application for systemic diseases and topically for different skin disorders. The MEs described herein can also be used as a drug delivery system to incorporate one or more water-soluble compounds of any type, including but not limited to small molecules and peptides, in a single ME.

Preparation of the microemulsion can be carried out under any suitable conditions as appropriate for an intended use. In one non-limiting embodiment, preparation of the microemulsion can be carried out at room temperature, in order to allow a water-soluble drug to dissolve fully in the pharmaceutically acceptable solvent. However, in other embodiments of the method, preparation of the microemulsion can be carried out at a temperature that is greater than room temperature. In aspects of this embodiment, preparation of the ME may be carried out at a temperature that is, e.g., greater than 21° C., greater than 25° C., greater than 30° C., greater than 35° C. or greater than 37° C., greater than 40° C., greater than 42° C., greater than 45° C., greater than 50° C., greater than 55° C., or greater than 60° C. In aspects of this embodiment, preparation of the ME may be carried out at a temperature that is between, e.g., about 20° C. to about 30° C., about 25° C. to about 35° C., about 30° C. to about 40° C., about 35° C. to about 45° C., about 40° C. to about 50° C., about 45° C. to about 55° C., or about 50° C. to about 60° C. In certain cases, preparation of the ME may be carried out at temperatures below room temperature, in order to allow a therapeutic to dissolve fully in solvent. However, in other embodiments of the method, preparation of the microemulsion can be carried out at a temperature that is less than room temperature, e.g., less than 10° C., greater than 5° C., greater than 0° C., greater than −10° C. or greater than −20° C.

In an embodiment, a water-soluble drug for use with an microemulsion is pregabalin. As used herein, “pregabalin” can refer to the following chemical structure:

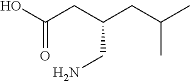

In another aspect are provided methods for treating glaucoma, reducing IOP, providing neuroprotection, treating eye injury, and preventing inherited retinal degenerations comprising administering to a subject in need thereof an amount effective to treat glaucoma, reduce IOP, provide neuroprotection, treat eye injury, and/or protect against inherited retinal degenerations of an inhibitor of Calcium Voltage-Gated Channel Auxiliary Subunit Alpha2Delta1 (CACNA2D1) protein. In one embodiment, the methods are to treat glaucoma; in one such embodiment, the glaucoma is primary open angle glaucoma (POAG). In an embodiment, the methods are to provide neuroprotection; in such embodiments the neuroprotection is from eye injury and/or retinal degeneration. For example, the eye injury can be a blast injury. For example, the retinal degenerations can be inherited retinal degenerations. In one embodiment, the inhibitor comprises a gabapentanoid, phenylglycine, or a pharmaceutically acceptable salt thereof. In another embodiment, the gabapentanoid comprises pregabalin, or a pharmaceutically acceptable salt thereof. In embodiments, the inhibitor can be pregabalin or an analog thereof. For example analogs can comprise gabapentin, gabapentin enacarbil, phenylalanine, amlodipine, 4-methyl pregabalin, acivicin, zoledronic acid, 5-Ethylhept-2-enoic Acid Ethyl Ester, 5-Ethyl-3-nitromethylheptanoic Acid Ethyl Ester, 4-(2-Ethylbutyl)pyrrolidin-2-one, 3-Aminomethyl-5-ethylheptanoic Acid Hydrochloride, 3-Ethylpentanenitrile, 3-Cyano-4-ethylhexanoic Acid Ethyl Ester, 3R-Aminomethyl-4R,5-dimethylhexanoic Acid, 4-(1-Ethylpropyl)pyrrolidin-2-one, 3-Aminomethyl-4-ethylhexanoic Acid Hydrochloride, 5S-Methyl-3-(4R-methyl-2-oxo-5S-phenyloxazolidine-3-carbonyl)hexanenitrile, (S)-2-(2-Aminoethyl)-4-methylpentanoic Acid, (4S)-Isobutyldihydrofuran-2-one, (4S)-Isobutyl-(3S)-methyldihydrofuran-2-one, (3S)-Azidomethyl-(2S,5)-dimethylhexanoic Acid Ethyl Ester, (4S)-Isobutyl-(3S)-methylpyrrolidin-2-one, (3S)-Aminomethyl-(2S,5)-dimethylhexanoic Acid Hydrochloride, 2,4-Dimethylvaleronitrile, 2-Ethyl-4-methylpentanenitrile, 3-Cyano-3,5-dimethylhexanoic Acid Ethyl Ester, 3-Cyano-3-ethyl-5-methylhexanoic Acid Ethyl Ester, 4-Methyl-4-isobutylpyrrolidin-2-one, 4-Ethyl-4-isobutylpyrrolidin-2-one, 3-Aminomethyl-3,5-dimethylhexanoic Acid, 3-Aminomethyl-3-ethyl-5-methylhexanoic Acid Hydrochloride, 3-Isobutyl-2-methyl-5-oxopyrrolidine-1-carboxylic Acid, tert-Butyl Ester, 2-Methyl-5-oxo-3-propylpyrrolidine-1-carboxylic Acid tert-Butyl Ester, 3-Butyl-2-methyl-5-oxopyrrolidine-1-carboxylic Acid tert-Butyl Ester, 3-(1-Amino-ethyl)-5-methylhexanoic Acid, 3-(1-Amino-ethyl)hexanoic Acid, 3-(1-Amino-ethyl)heptanoic Acid, 5-Methylhex-2-enoic Acid Ethyl Ester, 5-Methyl-3-(1-nitroethyl)hexanoic Acid Ethyl Ester, 4-Isobutyl-5-methylpyrrolidine-2-one, 3-Isobutyl-2-methyl-5-oxopyrrolidine-1-carboxylic Acid Benzyl Ester, 3-(1-Benzyloxycarbonylaminoethyl)-5-methylhexanoic Acid, 3-(1-Aminoethyl)-5-methylhexanoic Acid 4-Methylpent-2-enoic Acid, 3-(4-Methylpent-2-enoyl)-4R-phenyloxazolidin-2-one, 3-(3R,4-Dimethylpentanoyl)-4R-phenyloxazolidin-2-One, (3R)-(2-Acetoxy-1R-phenylethylcarbamoyl)-4S,5-dimethylhexanoic Acid tert-Butyl Ester, 2R-(1S,2-Dimethylpropyl)succinic Acid 4-tert-Butyl Ester, 4R-(1S,2-Dimethylpropyl)dihydrofuran-2-one, 3R-Bromomethyl-4S,5-dimethylhexanoic Acid Ethyl Ester, 3R-Azidomethyl-4S,5-dimethylhexanoic Acid Ethyl Ester, 4R-(1S,2-Dimethylpropyl)pyrrolidin-2-one, 3R,4S-3-Aminomethyl-4,5-dimethylhexanoic Acid, 3R-Benzyl-4R-isopropyldihydrofuran-2-one, 2R-Benzyl-3R-bromomethyl-4-methylpentanoic Acid Ethyl Ester, 2R-Benzyl-3R,4-dimethylpentanoic Acid Ethyl Ester, Acetic Acid 2R-benzyl-3R,4-dimethylpentyl Ester, 3R-Bromomethyl-4R,5-dimethylhexanoic Acid Ethyl Ester, 3R-Aminomethyl-4R,5-dimethylhexanoic Acid.

As used herein, the phrase “pharmaceutically acceptable salt” can refer to both pharmaceutically acceptable acid and base addition salts and solvates. Such pharmaceutically acceptable salts may be any salts suitable for an intended use, including but not limited to salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC—(CH₂)ₙ—COOH where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

As used here, a subject “in need thereof” can refer to a subject that has the disorder or disease to be treated or is predisposed to or otherwise at risk of developing the disease or disorder.

In one non-limiting example, the microemulsions can comprise or consist of the following components:
(a) a primary water-in-oil (w/o) phase constituting between about 0.1% and about 40% of the formulation, wherein the w/o phase comprises:
   (i) water at a concentration of between 0% and about 7% w/w of the formulation;
   (ii) oil at a concentration of between about 6% and about 13% w/w of the formulation;
   (iii) capryol 90 at a concentration of between about 1% and about 13% w/w of the formulation; and
   (iv) lecithin at a concentration of between about 1% and about 13% w/w of the formulation; and
(b) an external aqueous phase constituting 50-99.9% of the formulation, wherein the external aqueous phase comprises:
   (i) labrasol at a concentration of between about 0.1% and about 25% w/w of the formulation;
   (ii) cremophor EL at a concentration of between about 0.1% and about 25% w/w of the formulation;
   (iii) propylene glycol at a concentration of between 0% and about 45% w/w of the formulation; and
   (iv) water at a concentration of between about 10% and about 99.7% w/w of the formulation.

In another non-limiting example, the microemulsions can comprise or consist of the following components:
(a) a primary water-in-oil (w/o) phase constituting between about 0.1% and about 40% of the formulation, wherein the w/o phase comprises:
   (i) water at a concentration of between 2% and about 7% w/w of the formulation;
   (ii) oil at a concentration of between about 6% and about 9% w/w of the formulation;
   (iii) Capryol 90 at a concentration of between about 3% and about 9% w/w of the formulation; and
   (iv) lecithin at a concentration of between about 3% and about 9% w/w of the formulation; and
(b) an external aqueous phase constituting 50-99.9% of the formulation, wherein the external aqueous phase comprises:
   (i) labrasol at a concentration of between about 5% and about 9.5% w/w of the formulation;
   (ii) Cremophor EL at a concentration of between about 5% and about 9.5% w/w of the formulation;
   (iii) propylene glycol at a concentration of between 5% and about 25% w/w of the formulation; and
   (iv) water at a concentration of between about 30% and about 56% w/w of the formulation.

In another aspect are provided microemulsions designed as a drug delivery system for water-insoluble and sparingly-water soluble drugs molecules. In these embodiments, the microemulsions are the same as described above, but lack the internal aqueous phase and internal emulsifier. Thus, in this embodiment the ME comprises:
(a) a discontinuous (dispersed) oil phase; and
(b) an emulsifier encompassing the oil phase.

In one embodiment, the microemulsion further comprises (c) a continuous aqueous phase surrounding the emulsifier. In this embodiment, the oily drug solution is emulsified in the bioadhesive aqueous phase (such as a hydrogel as described herein) that contains a hydrophilic emulsifier (such as emulsifier with high HLB value).

In a further embodiment, the microemulsion comprises an insoluble or sparingly soluble drug in the discontinuous oil phase.

All embodiments disclosed above for the MEs can be used in this aspect as well, unless the context clearly dictates otherwise.

In embodiments, the microemulsion can contain antioxidants. For example, the antioxidants can comprise glutathione, tocopherol methoxypolyethylene glycol succinate (TPGS), sodium metabisulfite, alpha tocopherol, butylated hydroxyanisole (BHA), butylated hydroxytoluene (BHT), or a combination thereof.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Glaucoma is the leading cause of irreversible blindness in the world. This disease now affects more than 3 million people in the United States, and with the projected increased in longevity, this number can increase to ˜6.3 million by 2050. There are four major types of adult-onset glaucoma, all of which lead to vision loss through a final pathway of retinal ganglion cell (RGC) dysfunction and/or death. Each form of glaucoma can be associated with multiple and sometimes divergent risk factors, indicating there are multiple triggering mechanisms leading to RGC demise. For three of four adult-onset glaucoma sub-types, elevated intraocular pressure (IOP) is a predictive risk factor for visual field loss subsequent to RGC demise. The fourth sub-type, normal tension glaucoma, is not associated with high IOP and factors that trigger RGC death are unknown.

The current standard of care for adult-onset glaucoma includes treatment with IOP-lowering medications delivered topically as eye drops. The limitation of all currently FDA-approved glaucoma medications is limited efficacy. Specifically, IOP reduction does not prevent RGC death and resulting visual field loss in many glaucoma patients. In our studies, we used a systems genetics approach to identify a new IOP-lowering drug target, the calcium channel, voltage-dependent, α2δ1 subunit (aka CACNA2D1). Furthermore, we have identified a selective CACNA2D1 blocker, pregabalin (PRG), that exhibits IOP-lowering activity. We developed a topical extended release PRG microemulsion (ME) that increases drug entry into the eye, resulting in higher efficacy and duration of action. Our studies have uncovered an unanticipated additional benefit associated with PRG treatment for glaucoma. We have data to demonstrate that in addition to its localization to anterior segment structures that modulate IOP, CACNA2D1 is located in RGCs. We also have evidence to demonstrate that it directly protects against optic nerve (ON) damage in two animal models of glaucoma. Our findings indicate that our PRG ME can be a glaucoma therapy to both lower IOP and have direct, neuroprotective effects on RGCs.

Without wishing to be bound by theory our PRG ME will lower IOP with greater efficacy, as well as maintain the health of RGCs, a field of use for which there is currently no FDA-approved drug. This is supported by our data showing that CACNA2D1 is located in RGCs and their axons that comprise the NFL (nerve fiber layer) and ON and our extended-release bioadhesive ME enhances the movement of PRG into the eye after topical delivery.

The following tests are listed below:
1: We test whether PRG is a neuroprotectant for RGCs and the ON using once daily dosing.
2: We test whether PRG plays a direct role in RGC health by regulating the concentration of intracellular calcium (Ca²⁺).

Results: Based on data sets and without wishing to be bound by theory, the study will validate PRG ME as a neuroprotective therapy, which can minimize visual field loss of glaucoma patients.

### Example 2

Glaucoma is a complex, multifactorial, polygenetic disease that is the leading cause of irreversible blindness worldwide¹. Trends indicate that by 2040, as many as 111.8 million people worldwide will have glaucoma², and many of those will be legally blind due to optic nerve (ON) damage^{3,4}. Various subtypes of adult onset glaucoma-primary open angle (POAG), primary angle closure (PACG), and normal tension-share the clinical pathologies of retinal ganglion cell (RGC) and ON axonal damage, as well as subsequent visual field defects⁵. Several risk factors are known for this disease⁶⁻⁸, with elevated intraocular pressure (IOP) being a modifiable risk factor linked to the development and progression of glaucoma^{4,6,9-11}. The standard of care for all forms of adult-onset glaucoma is treatment with IOP-lowering medications delivered topically as eye drops. This treatment strategy does not prevent RGC/ON damage and resulting visual field loss in many glaucoma patients. As a result, there is interest in identifying molecules that offer direct neuroprotection or neuroenhancement to RGCs. Quotes from ophthalmologists include: “neuroprotection is a big void”; “there are no therapeutic options for neuroprotection”; and “lowering IOP is not enough; we need to add neuroprotection to increase the efficacy of glaucoma meds.”

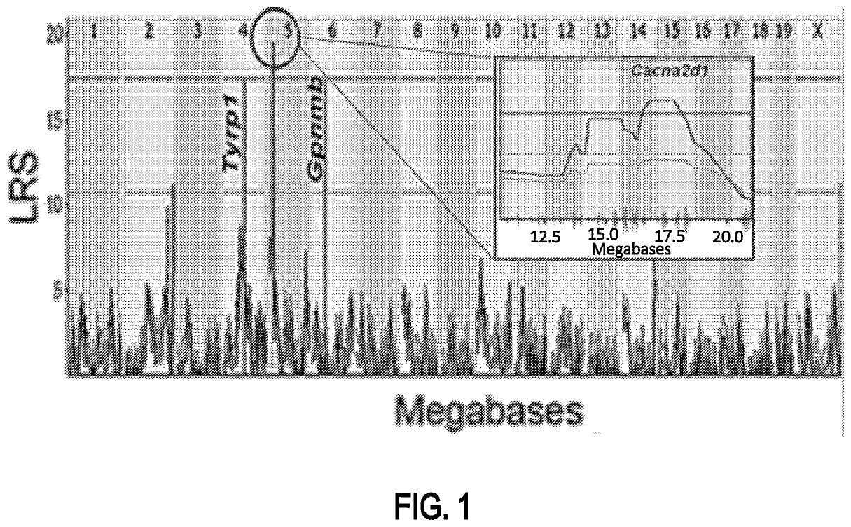

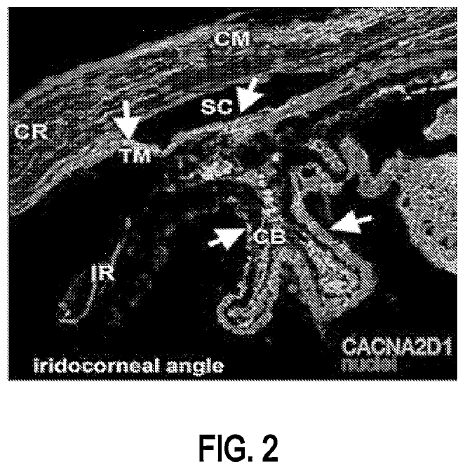

Identification of a new IOP-lowering drug with direct neuroprotective action: Although IOP reduction is the therapeutic option for all forms of adult onset glaucoma, unfortunately, current IOP-lowering medications do not address underlying pathologies or genetic variations. By combining a forward murine genetics approach with cell biology, pharmacology and analysis of human GWAS data, we identified a new gene that modulates IOP—for example, the calcium channel, voltage-dependent, α2δ1 subunit (aka Cacna2d1)¹² (FIG. 1). Our approach combined IOP measurement of 65 recombinant, inbred BXD mouse lines across five age cohorts with genetic polymorphism assessment, and whole eye microarray analyses. As a bidirectional component of our study, we corroborated an imputed SNP in CACNA2D1 within a human POAG population. Immunohistochemistry studies in humans, rabbits and mice^{12,13} indicate that CACNA2D1 is located in the ciliary body (CB) and outflow structures, which can influence IOP (FIG. 2). Pharmacology studies demonstrated that topical dosing with pregabalin (PRG), an antagonist with specificity for CACNA2D1, lowers TOP in a dose dependent manner¹³. These findings support the development of PRG as representative of a new class of glaucoma therapeutics with a mechanism of action distinct from current TOP-lowering medications.

An additional unexpected finding from our studies shows that CACNA2D1 is also expressed in RGCs. Based on these data, and without wishing to be bound by theory, CACNA2D1 represents a druggable target on RGCs and the ON to protect against glaucoma-induced neurodegeneration, in addition to its IOP-lowering effects.

We prepared, optimized, and characterized an extended-release microemulsion (ME)-based formulation for delivery of PRG to the eye. Our ME was designed to overcome the drawbacks associated with aqueous eye drops that include rapid drainage, short corneal contact time and minimal corneal penetration; all of which lead to reduced efficacy and poor patient adherence. We accomplished this by engineering a multilayered ME using biocompatible components with in situ gelling properties that improve bioadhesion, enhance corneal penetration and provide continuous release of drug for up to 30 hours. Because our ME has a small particle size (<20 nm), it is transparent and does not blur vision. There is a dose response reduction in TOP to increasing doses of PRG ME, with 0.6% PRG being the minimal dose that provides the maximum effect with a reduction in IOP of nearly 40% that does not return to baseline until 34 hours after dosing (test 1;¹³. We also determined that daily dosing with PRG ME for 21 days did not result in a decrease in efficacy (aka tachphylaxis) of the formulation (test 2;¹³). Throughout the study, TOP remained at a reduced level in the treated eye, while the control eye remained at the elevated baseline TOP. We determined the distribution of PRG in the eye and body after the conclusion of the tachyphylaxis study (test 2;¹³). After 21 consecutive days of topical dosing with our PRG ME, we determined that the drug reached the tissues of the posterior eye, including the retina at a concentration sufficient to inhibit CACNA2D1. The control eye that received blank ME contained miniscule levels of PRG, as did plasma and all peripheral organs. Slit lamp and histopathological exams determined that the drug-loaded ME is safe and tolerated by the eye¹³. These are significant findings because they demonstrate that our ME can deliver hydrophilic molecules to the retina. This expands the use of this drug delivery system to include diseases of the posterior pole, including delivery of neuroprotective molecules to RGCs. We directly test whether, in addition to being an effective IOP-lowering therapeutic, PRG, when delivered by our ME, is also a direct neuroprotectant to RGCs.

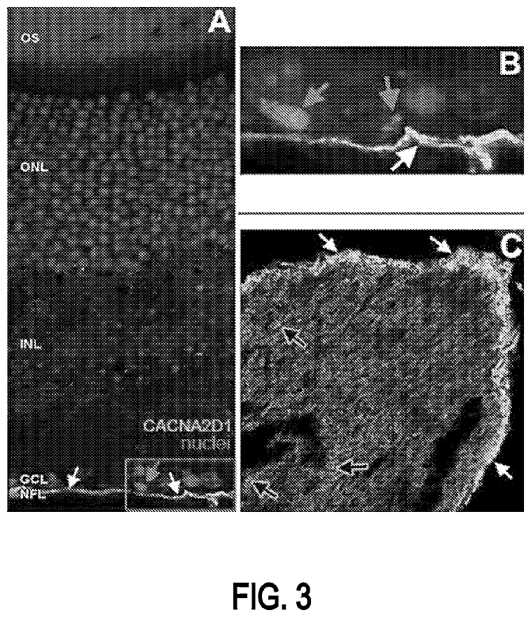

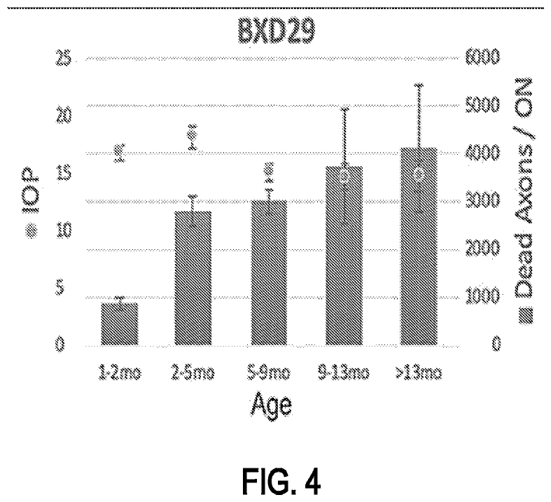

Concepts and approaches:
We have identified a new IOP-lowering drug PRG that was selected based on our discovery of Cacna2d1 as an IOP-modulating gene¹². PRG has high affinity and specificity for CACNA2D1. All current therapies have limitations including lack of sustained action, eye irritation, as well as other side effects and drug interactions. Changing therapies for patients who experience these side effects is challenging because all current therapies are linked to a restricted set of mechanisms of action. Therefore, available treatment options are limited. PRG represents a new class of glaucoma drug that acts through a new, previously unknown target and mechanism of action, thus providing an option for many patients. PRG has been approved by the FDA and is currently a generic, which allows us to repurpose it through the 505(b)(2) regulatory pathway and provide a new class of glaucoma therapies.
We have prepared and characterized an extended-release, multilayered, bioadhesive, topical ME-based formulation for delivery of hydrophilic drugs to the eye. Our ME was designed to overcome the drawbacks with traditional eye drops that include rapid drainage, short corneal contact time and minimal corneal penetration, all of which lead to reduced efficacy and poor patient adherence. The improved efficacy is supported by increased mucoadhesive properties, increased corneal permeability, the miniscule particle size, penetration enhancing ability and sustained release.** When delivered by our ME, PRG movement into the eye is enhanced and PRG can be detected in the retina, thus allowing for topical delivery of a retina-targeted therapy.
We have data that demonstrates that CACNA2D1 is also localized to RGC cell bodies and their axons which comprise the ON (FIG. 3). Additional data indicates that PRG prevents degeneration of axons in the ON even in the absence of a reduction in IOP (FIGS. 4-6). Collectively these data indicate that PRG is a direct neuroprotectant for RGCs, and will limit visual field loss, beyond IOP lowering alone. Presently there are no FDA-approved neuroprotective therapies for glaucoma. Our PRG ME would be the first glaucoma therapy to both lower IOP, and protect RGCs and the optic nerve.

Without wishing to be bound by theory, PRG is a neuroprotectant for RGCs and the ON using once daily topical dosing.

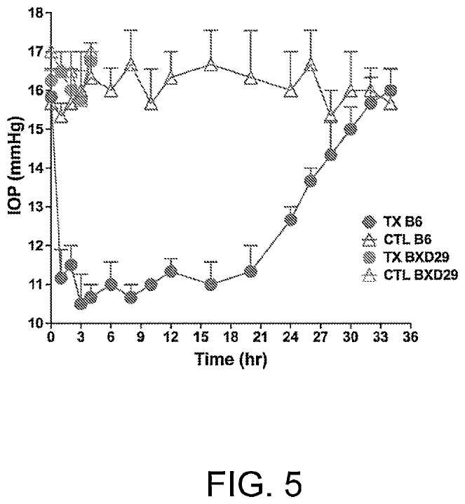

Objective: An obstacle in this study is the in vivo assessment of any direct neuroprotective effects of PRG that are independent of IOP lowering. To address this challenge, we drew on our data demonstrating that mice with the D haplotype of Cacna2d1 are non-responsive to the IOP-lowering properties of PRG¹². In addition, we mined our databases of IOP and ON damage gathered from BXD mice across 5 age cohorts^{12,13} and identified BXD29 as a strain that carries the D haplotype of Cacna2d1; has an IOP that does not change with age (FIG. 4, orange circles), yet has marked ON damage at an early age (FIG. 4, blue bars); and its IOP is unresponsive to PRG treatment (FIG. 5, red symbols). These characteristics make BXD29 ideal for evaluating the direct neuroprotective properties of PRG on RGCs by eliminating indirect effects that would be mediated through IOP reduction.

Non-limiting exemplary data: We made a discovery that can make PRG effective in treating vision loss in glaucoma. In addition to its presence in the anterior segment (FIG. 2), CACNA2D1 is also localized to RGCs (FIGS. 3A&B), as well as the ON (FIG. 3C). PRG can ameliorate injury to multiple neuronal cell types in brain¹⁶⁻²¹, our data indicate that CACNA2D1 can be a target for both lowering of IOP and direct neuroprotection of RGCs/ON.

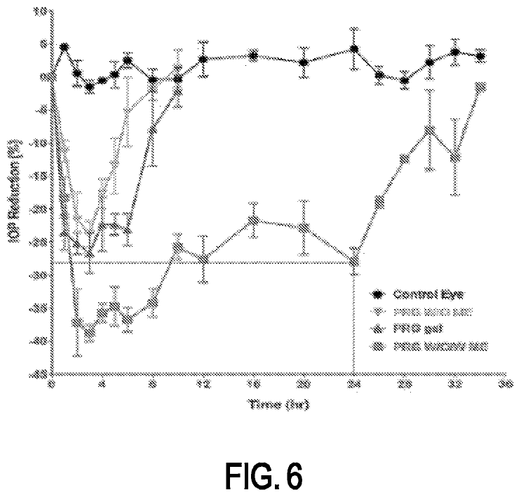

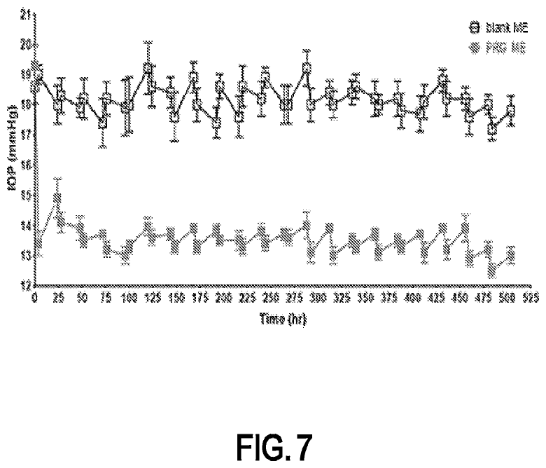

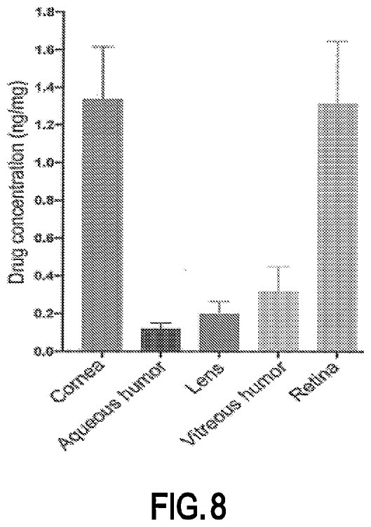

We have developed a multilayered microemulsion (ME) with extended release properties that supports once daily dosing of PRG for IOP reduction, a single drop of which induces a marked reduction in IOP (42%) that returns to baseline at 33 hrs (AUC=170 mmHg·hr; red squares Dutch belted rabbits; FIG. 6)¹³. At 24 hours after dosing, IOP is still reduced by >20%. In the absence of our ME, PRG produces only a 30% IOP reduction that returns to baseline after 10 hrs (AUC=38 mmHg·hr) demonstrating that our formulation enhanced the IOP-lowering efficacy and duration of action of PRG. Moreover, it is safe and tolerated in the eye¹³. Once daily dosing for 21 days demonstrates that there is no drug tolerance and that IOP never returns to its elevated pre-dosing baseline (red; Dutch belted rabbits; FIG. 7)¹³. For our purposes of evaluating PRG as a direct neuroprotectant on RGCs and the ON, topical delivery of PRG via our ME also enhances drug penetration into the eye allowing for drug levels to reach the retina the site of RGCs and their axons (Dutch belted rabbits; FIG. 8)¹³.

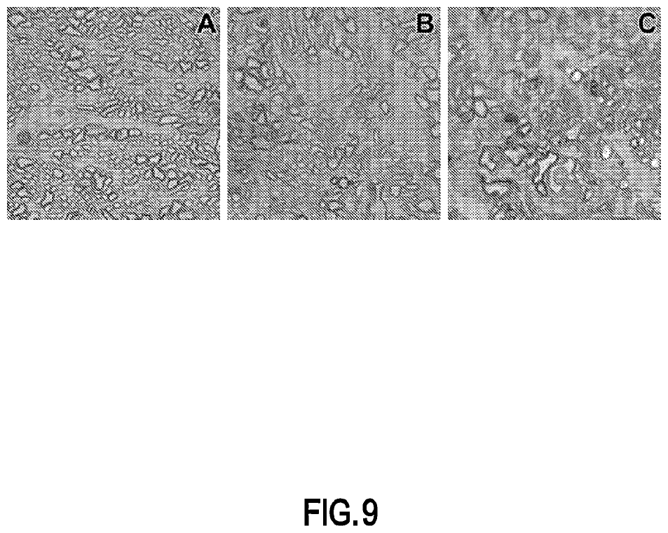

We have evidence to show, without wishing to be bound by theory, that topical dosing with our PRG ME is neuroprotective to RGC axons in the ON. We dosed BXD29 mice daily with PRG ME beginning at 5 weeks of age (FIG. 9A) before ON degeneration was marked. In ONs from BXD29 mice dosed daily for 6 weeks, ON axons were preserved at near normal levels (FIG. 9B). In contrast, ON from mice dosed with unmedicated ME had loss of axon profiles and increased areas of debris (FIG. 9C). The studies will evaluate the role of PRG as a RGC neuroprotectant and define its cellular mechanism of action.

Research Design and Methods: Our test subjects can be BXD29 (JAX #010981) for the reasons outlined above, and C57Bl/6J (B6; JAX #000664; control with no ocular pathology). Using the power calculations of John et al. in which they evaluated the neuroprotective effects of nicotinamide in the D2 glaucoma model²², and without wishing to be bound by theory, 80 mice will be enrolled in this study, balanced equally between males and females. In the prophylactic arm of this study, BXD29 mice will be aged to 1 mo, prior to the onset of ON damage (FIG. 9). Both eyes of each mouse will be topically dosed daily with 5 μl of PRG ME²³⁻²⁵ for 3 months until mice are 4 months of age and ON degeneration would become significant in the absence of PRG (n=20). A second set of mice will receive blank ME in both eyes (n=20; same dosing pattern). A third set will receive no intervention and will be used as naïve controls (n=20 each BXD29; 10 each will be used to obtain baseline data, and 10 each will be used to obtain terminal data). A fourth set will include B6 mice (n=20) as a control with no ocular pathology. They will be examined in parallel with the third set as naïve controls. We have determined that our formulation is stable for a minimum of 3 months at 5° C., therefore a single batch of PRG ME will be prepared¹³, aliquoted into individual 4 ml opaque dropper bottles and coded for each mouse. Bottles will be stored at 5° C. for the duration of this investigation. Experimenter will be blinded to the contents of formulation bottle. To characterize the neuroprotective effect of PRG, we will use the following clinical and laboratory examinations:
Clinical exams (baseline and monthly): IOP measurement; scotopic electroretinogram (ERG) including scotopic threshold response and pattern ERGs (PERGs) which are indicators of RGC function; visual acuity (VA) and contrast sensitivity (CS); and OCT imaging through the optic nerve head (ONH) using our published methods^{13,23,25-27}.
Laboratory exams (baseline and terminal): morphological and immuno/histochemical assessment of the retina (including γ-synuclein (SNCG), brain-specific homeobox/POU domain protein 3A (BRN3A), and TUNEL staining); and histological assessment of ON damage using our published methods^{12,28-31}.
Statistical Analyses: Statistical differences will be analyzed by one-way ANOVA followed by Tukey-Kramer multiple comparisons test (in the event of a significant F-test) using GraphPad Prism-8 statistical software.

The outcome described herein will be the determination of the neuroprotective nature of PRG when applied prophylactically. In BXD29 mice treated with PRG ME, compared to untreated mice, we will measure: near baseline ERG and PERG amplitudes; near baseline VA & CS levels; fewer TUNEL-positive RGCs; more SNCG- & BRN3A-immunopositive RGCs; fewer necrotic axons and areas of scarring in the ON. There are no FDA-approved treatments that act directly on RGCs to block or mitigate visual field loss in glaucoma patients. Without wishing to be bound by theory, PRG is neuroprotective, our PRG ME would be the first glaucoma therapy with multiple mechanisms of action IOP-lowering and neuroprotection.

The performance will include the demonstration that retinal and RGC structure, function and protein expression patterns are maintained at near baseline levels after 3 months of daily dosing with our PRG ME, in the absence of effect on IOP.

Without wishing to be bound by theory, PRG plays a direct role in RGC health by regulating the concentration of intracellular (Ca²⁺) levels.

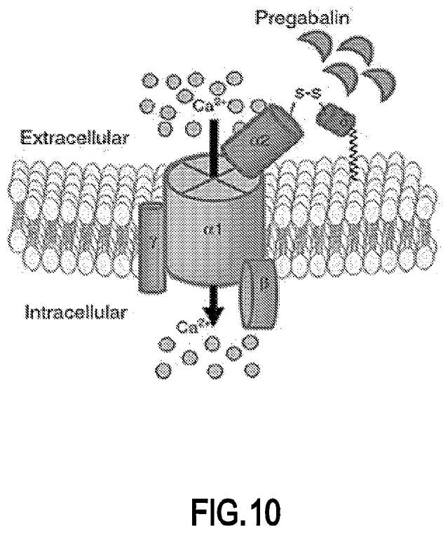

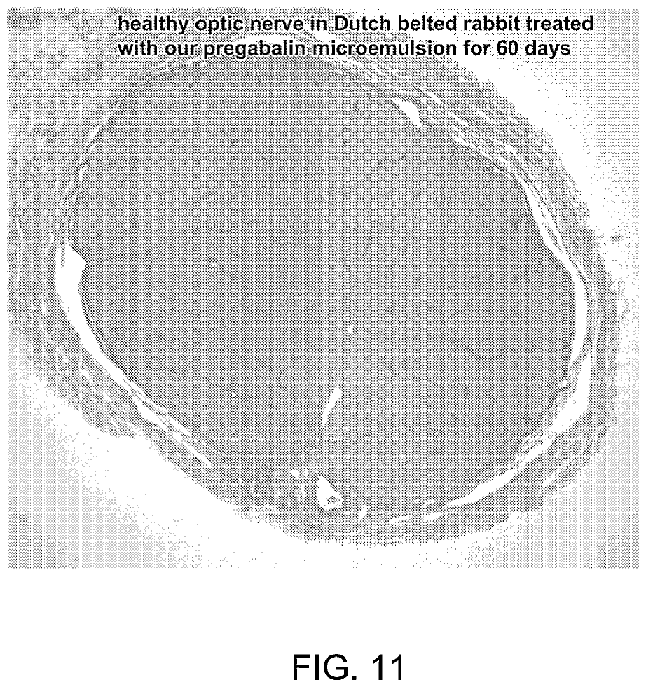

Non-limiting, exemplary objective: The mechanism of action by which PRG can function as a neuroprotective agent to RGCs is unknown. However, given that PRG is a selective CACNA2D1 blocker 1 and CACNA2D1 is localized to RGCs and their axons (FIG. 3), the first step of the mechanism of action by which PRG is neuroprotective is by attenuating the influx of ionic Ca²⁺ across the cell membrane (FIG. 10).

Non-limiting, exemplary research design: We will measure Ca²⁺ signals mediated by voltage-gated Ca²⁺ channels (VGCC) in RGC cell bodies and axons from BXD29 and C57Bl/6J (B6) mice after loading with a Ca²⁺ indicator dye using established methods. BXD29, with early-onset ON damage (FIG. 4), is the same strain of mice that we use in test 1 (along with the same three experimental conditions), while B6 mice will serve as control mice with no ON damage. Mouse eye cups from each experimental condition (with an ON stump ˜1 mm long) will be obtained. A mixture of a Ca²⁺-sensitive dye (Fluo-5F, pentapotassium salt; 40 mM in H₂O) and a Ca²⁺-insensitive dye (Alexa 568, 20 mM in H₂O) will be injected into the ON stump using a Hamilton syringe. This strategy will allow us to use a ratiometric approach to calculate the amplitude of free Ca²⁺ signal in RGCs and their axons. Because the molecular weights of Fluo-5F and Alexa 568 are similar, they will diffuse into the cell at roughly the same rate³². Eyecups will be placed in mammalian Ringer solution bubbled with 95% O₂/5% CO₂ for 1 hr in the dark to allow the dye to load into RGCs and their axons. Retinas will be isolated, divided into quadrants, mounted RGC-side down on a glass slide using harp slice grids for stabilization, and imaged using an Olympus FV3000-RS confocal microscope. A six-channel gravity superfusion system (Warner Instruments) will be used to deliver solutions bubbled with 95% O₂/5% CO₂ at physiological temperature. RGCs and RGC axons will be depolarized by raising the extracellular potassium concentration ([K⁺]) from 3 mM to 60 mM for 33 s to activate VGCCs 33,34. Superfusion of elevated ([K⁺] and imaging will be automated and synchronized using a transistor-transistor logic (TTL) digital signal^{35,36}. Osmolarity will be maintained constant in the high K⁺ solution by reducing sodium (Na⁺). We will apply the Na⁺ channel blocker tetrodotoxin (TTX; 200 nM), to determine the extent to which Na⁺ channels contribute to the generation of [Ca²⁺] transients in RGC somata during high K⁺ stimulation. Fluorescent intensity values from ROIs will be averaged within an experimental condition, each considered an independent observation for statistical testing using Student's unpaired t-test. Correlations will be made between the clinical and laboratory outcomes in test 1 and the Ca²⁺ transient amplitudes obtained in test 2.

Non-limiting, exemplary results: The outcome will be the measurement of Ca²⁺ transients in the presence/absence of PRG in a model of normal tension glaucoma (BXD29). These outcomes will allow us to determine the mechanism of action of PRG as a neuroprotective therapeutic to RGCs and their axons. The amplitude of normalized Ca²⁺ transients from BXD29 mice treated prophylactically with PRG will be similar to B6 mice or naïve BXD29 mice at baseline (1 month of age). Furthermore, because RGCs and axons of untreated BXD29 mice are damaged at an early age (FIG. 5), the evoked Ca²⁺ transients obtained from naïve BXD29 or untreated mice at 4 months of age will be larger than both B6 and BXD29 mice treated with PRG. Without wishing to be bound by theory, positive indicators of RGC health will be inversely correlated with the amplitude of the Ca²⁺ transients measured in each condition.

Alternative approaches: The methodology and approaches for this project are established and understood by the skilled artisan. If we do not find changes in the Ca²⁺ influx from RGCs and axons of untreated BXD29 mice in whole mount preparation, we will isolate RGCs to compare evoked Ca²⁺ signals through VGCCs. To do so, we will perform whole cell voltage clamp on an isolated RGC combined with calcium imaging after loading a Ca²⁺ indicator via patch pipette.³⁴ Further, we will test whether CACNA2D1 expression is reduced in BXD29 mice treated prophylactically with PRG. To test this, we will conduct semi-quantitative immunohistochemistry and perform higher resolution imaging to compare the expression of VGCC subunits.

A milestone will be the demonstration that a) PRG has role on CACNA2D1 in RGCs and axons, and b) PRG reduced Ca²⁺ signals in RGCs and axons similar to B6 mice.

### REFERENCES CITED IN THE EXAMPLE

1 Standefer, J. E. In focus: Glaucoma is second leading cause of blindness globally. Bull World Health Organ 82, 887-888 (2004).
2 Tham, Y.-C. et al. Global Prevalence of Glaucoma and Projections of Glaucoma Burden through 2040. Opthalmology 121, 2081-2090 (2014).
3 Bourne, R. R. A. Worldwide glaucoma through the looking glass. Br J Ophthalmol 90, 253-254 (2006).
4 Quigley, H. A. & Broman, A. T. The number of people with glaucoma worldwide in 2010 and 2020. Br J Ophthalmol 90, 262-267 (2006).
Allingham, R. R., Liu, Y. & Rhee, D. J. The genetics of primary open-angle glaucoma: A review. Experimental Eye Research 88, 837-844, doi:10.1016/j.exer.2008.11.003 (2009).
6 Gordon, M. et al. The Ocular Hypertension Treatment Study: baseline factors that predict the onset of primary open-angle glaucoma. Arch Ophthalmol 120, 714-720 (2002).
7 Libby, R., Gould, D., Anderson, M. & John, S. W. Complex genetics of glaucoma susceptibility. Ann Rev Genomics Hum Genet 6, 15-44 (2005).
8 Quigley, H. A. Glaucoma: macrocosm to microcosm: The Friedenwald Lecture. Invest Ophthalmol Vis Sci 46, 2662-2670 (2005).
9 Armaly, M. et al. Biostatistical analysis of the collaborative glaucoma study. I. Summary report of the risk factors for glaucomatous visual-field defects. Arch Ophthalmol 98, 2163-2171 (1980).
10 Leske, M., Connell, A., Wu, S., Hyman, L. & Schachat, A. Risk factors for open-angle glaucoma. The Barbados Eye Study. Arch Ophthalmol 113, 918-924 (1995).
11 Sommer, A. et al. Relationship between intraocular pressure and primary open angle glaucoma among white and black Americans. Arch Ophthalmol 109, 1090-1095 (1991).
12 Chintalapudi, S. R. et al. Systems genetics identifies a role for Cacna2d1 regulation in elevated intraocular pressure and glaucoma susceptibility. Nat Commun 8, 1755 (2017).
13 Ibrahim, M. et al. Once Daily Pregabalin Eye Drops for Management of Glaucoma. ACS Nano 13, 13728-13744 (2019).
14 Wittenborn, J. & Rein, D. in Prevent Blindness America (2013).
15 National Eye_Institute. Glaucoma: NEILooks Ahead, <http://nei.nih.gov/health>(
16 Aşci, S., Demirci, S., Aşci, H., Do\check{g}uç, D. & Onaran, I. Neuroprotective Effects of Pregabalin on Cerebral Ischemia and Reperfusion. Balkin Med J 33, 221-227 (2016).
17 Kazanci, B. et al. Neuroprotective Effects of Pregabalin Against Spinal Cord Ischemia-Reperfusion Injury in Rats. Turk Neurosurg 27, 952-961 (2017).
18 Moriya, S. et al. Neuroprotective effects of pregabalin in a rat model of intracisternal facial nerve avulsion. J Neurosurg Sci 61, 495-503 (2017).
19 Abraham, M., Faisal, K., Biju, C. & Babu, G. Neuroprotective effect of lacosamide and pregabalin on strychnine induced seizure models in rat. World J Pharm Pharmaceut Sci 3, 1324-1329 (2014).
20 Song, Y. et al. Effect of pregabalin administration upon reperfusion in a rat model of hyperglycemic stroke: Mechanistic insights associated with high-mobility group box 1. PLOS One 12, e0171147 (2017).
21 Jorige, A. & Annapurna, A. Neuroprotective Effect of Lacosamide and Pregabalin on Pentylenetetrazole Induced Seizure Models in Rat. Br Biomed Bull (2014).
22 Williams, P. et al. Vitamin B3 modulates mitochondrial vulnerability and prevents glaucoma in aged mice. Science 355, 756-760 (2017).
23 Ibrahim, M., Abd-Elgawad, A., Soliman, O. & Jablonski, M. Novel topical ophthalmic formulations for management of glaucoma. Pharm Res 30, 2818-2831 (2013).
24 Ibrahim, M., Abd-Elgawad, A., Soliman, O. & Jablonski, M. Nanoparticle-based topical ophthalmic formulations for sustained celecoxib release. J Pharm Sci 102, 1036-1053 (2013).
25 Ibrahim, M., Abd-Elgawad, A., Soliman, O. & Jablonski, M. Natural bioadhesive biodegradable nanoparticles-based topical ophthalmic formulations for sustained celecoxib release: In vitro Study. Pharm Technol & Drug Res 2, 7 http://dx.doi.org/10.7243/2050-7120X-7242-7247 (2013).
26 Swaminathan, S. et al. Novel Endogenous Glycan Therapy For Retinal Diseases: Safety, In Vitro Stability, Ocular Pharmacokinetic Modeling and Bio-distribution. AAPS Journal 16, 311-312 (2014).
27 Ibrahim, M., Abd-Elgawad, A., Soliman, O. & Jablonski, M. Natural Bioadhesive Biodegradable Nanoparticle-Based Topical Ophthalmic Formulations for Management of Glaucoma. Transl Vis Sci Technol 4, 12 (2015).
28 Jablonski, M. et al. The Tennessee Mouse Genome Consortium: identification of ocular mutants. Vis Neurosci 22, 595-604 (2005).
29 Chintalapudi, S. et al. Isolation and Molecular Profiling of Primary Mouse Retinal Ganglion Cells: Comparison of Phenotypes from Healthy and Glaucomatous Retinas. Front Aging Neurosci 8, 93 (2016).
30 Chintalapudi, S., Morales-Tirado, V., Williams, R. & Jablonski, M. Multipronged Approach to Identify and Validate a Novel Upstream Regulator of Sncg in Mouse Retinal Ganglion Cells. FEBS J 283, 678-693 (2015).
31 Stiemke, A. et al. Systems Genetics of Optic Nerve Axon Necrosis During Glaucoma Front Genet 11, 31 (2020).
32 Van Hook, M. J. & Thoreson, W. B. Weak endogenous Ca2+ buffering supports sustained synaptic transmission by distinct mechanisms in rod and cone photoreceptors in salamander retina. Physiol Rep 3, doi:10.14814/phy2.12567 (2015).
33 Vaithianathan, T., Akmentin, W., Henry, D. & Matthews, G. The ribbon-associated protein C-terminal-binding protein 1 is not essential for the structure and function of retinal ribbon synapses. Mol Vis 19, 917-926 (2013).
34 Vaithianathan, T. & Matthews, G. Visualizing synaptic vesicle turnover and pool refilling driven by calcium nanodomains at presynaptic active zones of ribbon synapses. Proc Natl Acad Sci USA 111, 8655-8660, doi:10.1073/pnas.1323962111 (2014).
35 Vaithianathan, T., Henry, D., Akmentin, W. & Matthews, G. Nanoscale dynamics of synaptic vesicle trafficking and fusion at the presynaptic active zone. Elife 5, doi:10.7554/eLife.13245 (2016).
36 Vaithianathan, T., Wollmuth, L. P., Henry, D., Zenisek, D. & Matthews, G. Tracking Newly Released Synaptic Vesicle Proteins at Ribbon Active Zones. iScience 17, 10-23, doi:10.1016/j.isci.2019.06.015 (2019).

### Example 3

Glaucoma is the leading cause of irreversible blindness in the world, affecting more than 3 million people in the United States alone, with a projected annual increase in incidence of 4.1%. There are four major types of glaucoma, all of which lead to vision loss through a final pathway of retinal ganglion cell (RGC) dysfunction and/or death. For a form of glaucoma (primary open angle glaucoma, POAG), the most significant risk factor is elevated intraocular pressure (IOP). The standard of care for all adult-onset glaucoma currently consists of treatment with one or more of the four classes of IOP-lowering medications (e.g., beta-blockers, carbonic anhydrase inhibitors, sympathomimetics, miotics or prostaglandin derivatives) delivered topically as eye drops. These medications lower IOP by decreasing the volume of aqueous humor either through inhibition of its production by the ciliary body or enhancement of its drainage through the outflow structures. No current therapy affects both production and drainage. Other limitations of available medications are their short half-lives and limited residence time on the cornea, resulting in the need for up to 3 doses per day. The requirement for multiple applications of eye drops daily, along with associated eye irritation, orbital side effects (such as conjunctival hyperemia), and systemic side effects, collectively contribute to the low level of patient compliance/adherence documented by many ophthalmologists.

We used a systems genetics approach to identify a new IOP-lowering drug target, the calcium channel, voltage-dependent, α2δ1 subunit (aka CACNA2D1). Furthermore, we identified a selective CACNA2D1 blocker, pregabalin (PRG), that exhibits dual IOP-lowering activity linked to both IOP production and drainage. We developed a topical extended release PRG formulation that increases drug entry into the eye, resulting in higher efficacy and longer duration of action, with minimal eye irritation or ocular side effects. Our overall objective with this study is to further develop PRG as representative of a new class of glaucoma therapeutics with a mechanism of action that is distinct from current IOP-lowering medications. We will focus on CACNA2D1 as a druggable target in the anterior segment with the intent of optimizing an efficacious IOP-lowering treatment that lowers IOP by ˜40% and maintains it at a reduced level for 34 hours after a single topical dose. PRG is an FDA approved drug being repurposed as a glaucoma therapeutic, without wishing to be bound by theory PRG-when formulated in a topical microemulsion with characteristics of sustained release, bioadhesion, and corneal penetration enhancement-will elicit an IOP-lowering response that is equal to or greater in amplitude and longer in duration, than other glaucoma medications currently on the market.

Without wishing to be bound by theory we will complete our formulation studies, scale up GLP production of drug product, and complete toxicology studies for a new glaucoma drug with a new mechanism of action and sustained IOP lowering properties. This treatment strategy will reduce the burden to glaucoma patients and lead to better visual outcomes. To address our objective, we will perform the following:
1: Increase the stability, while maintaining efficacy, of a new topically applied microemulsion formulation.
2: Manufacture drug product under Good Laboratory Practice (GLP) conditions for toxicology studies.
3: Perform toxicology studies with guidance from the Food and Drug Administration (FDA).

Without wishing to be bound by theory, PRG also provides neuroprotection to RGCs. In that study, we will establish the mechanism of action for neuroprotection and its sustainability achieved through our microemulsion eye drop formulation. Completion of that study will allow us to expand our overall drug development program for an IOP-lowering drug with a mechanism of action to include an additional neuroprotective benefit.

### Example 4

Glaucoma continues to be the leading cause of irreversible blindness in the world¹ in spite of available treatment options. Trends indicate that by 2040, as many as 111.8 million people worldwide will have glaucoma¹, and many of those will be legally blind due to optic nerve (ON) damage¹. Various subtypes of adult onset glaucoma primary open angle (POAG), primary angle closure (PACG), and normal tension-share the common clinical pathologies of retinal ganglion cell (RGC) and ON axonal damage, as well as subsequent visual field defects². Several risk factors are known for this disease³⁻⁵, with elevated intraocular pressure (IOP) being the only modifiable risk factor linked to the development and progression of glaucoma^{3,6-9}. As such, the standard of care for all forms of adult-onset glaucoma is treatment with IOP-lowering medications delivered topically as eye drops. The market validation research we conducted has identified multiple issues with current glaucoma medications, some of which are: 1) lack of sustained action; 2) requirement for multiple daily dosing; 3) direct ocular irritation; 4) systemic and ocular side effects for individual patients; and 5) limited options for drugs with different mechanisms of action (MOA). We have identified a new druggable target for treatment of glaucoma, as well as a drug and formulation to address many of these pain points.

IOP-lowering via a new MOA using a repurposed therapeutic: By combining a murine genetics approach with cell biology, pharmacology, and analysis of human genome wide association database (GWAS) data, we identified a genetic locus that significantly modulates IOP namely the calcium channel, voltage-dependent, α2δ1 subunit (aka Cacna2d1)¹⁰ (FIG. 1). As a bidirectional component of our study, we corroborated an imputed single-nucleotide polymorphism (SNP) in CACNA2D1 within a human POAG population. Immunohistochemistry studies in humans, rabbits, and mice indicate that CACNA2D1 is located in the ciliary body (CB) and outflow structures (FIG. 2), both of which can influence TOP. To validate the identified target, we demonstrated that topical dosing with pregabalin (PRG), an antagonist that has high specificity for CACNA2D1 and blocks voltage-dependent calcium (Ca²⁺) flux through the Caᵥα1 pore of L-type Ca²⁺ channels¹¹, lowers IOP in a dose dependent manner in Dutch belted rabbits¹². These findings are consistent with earlier findings that implicated Ca²⁺ flux as a regulator of IOP through actions in both the CB and outflow structures¹³, This is highly significant because few approved IOP-lowering drugs act on both the inflow and outflow facilities, and that we can be able to clarify the implicated but yet unproven role of Ca²⁺ flux on inflow and outflow control. On the therapeutic side, because the amplitude and duration of PRG-mediated IOP reduction in our studies were limited (17% IOP reduction that returns to baseline at 6 hr)¹⁵, we then engineered a new topical extended release microemulsion (ME) formulation to increase PRG efficacy and duration of action 1. Our data demonstrate that targeting CACNA2D1 with a single drop of our latest PRG ME formulation applied to the cornea effects a sustained reduction of IOP of up to at least 24 hours with significantly better efficacy than the initial formulation; and hence it supports once daily dosing of PRG for IOP reduction (FIG. 3; a maximal 42% IOP reduction that returns to baseline at 33 hr), making our PRG ME equal or superior to Timolol and Xalatan. These findings indicate the development of PRG as a new class of glaucoma therapeutics with a MOA distinct from current approved IOP-lowering medications.

Our discovery of the IOP-lowering effects of PRG and its specific localization to structures that regulate IOP (the ciliary body (CB) and outflow structures), together with a new delivery system to provide extended duration of action topically applied PRG represents a strategy for development of both a new treatment and a new delivery system for decreasing IOP in glaucoma. PRG is a gabapentinoid drug, yet it does not bind to γ-aminobutyric acid (GABA) receptors, nor does it affect GABA release or uptake¹⁶. Rather, it binds with high affinity to CACNA2D1 and provides analgesia via a mechanism that does not involve anti-inflammatory pathways. PRG has been widely used in treating epileptic seizures or neuropathic pain disorders such as fibromyalgia¹⁷ with a safe profile¹⁸. Potential additive behavior in some susceptible populations and rare hypersensitivity such as angioedema have been reported^{18,19}. Because the dose of PRG in a drop (50 μL) of our ME is >2000-fold less than in tablets or suspension and the systemic exposure from that topical dose can be even much less, oral dose side effects will not be of concern in our topical administration.

Calcium channel blockers (CCBs) as glaucoma therapies: Why they failed and implications for future studies: Voltage-gated calcium channels regulate calcium (Ca²⁺) influx and are distributed throughout the body. Multiple subunits, including CACNA2D1, comprise the channel and serve to modulate the activity of the Caval pore (FIG. 10). Because previous studies have demonstrated a link between Ca² and glaucoma^{20,21}, systemic CCBs that target the Caval pore (e.g., verapamil) have been evaluated as therapies for POAG. However, the outcomes of these investigations have been inconsistent, with some studies demonstrating that CCBs are effective in lowering IOP and improving visual function, with others failing to replicate those results²²⁻²⁸. Systemic CCBs also have significant side-effects such as dizziness or headache²²⁻²⁸. No previous study has evaluated glaucoma-linked auxiliary modulators of the Ca²⁺ channel such as our system genetic-identified target, CACNA2D1 (FIG. 10). In addition to the genetic rationale and the advantages of repurposing an approval and widely used drug for using PRG over traditional CCBs to lower IOP, there are multiple physiochemical characteristics, such as high aqueous solubility, low partition coefficient, and high membrane permeability that makes PRG a useful topical therapeutic option over CCBs. These characteristics allow PRG to cross the epithelial layer of the cornea, yet remain soluble in the aqueous environment of the corneal stroma and AH, so it can reach the target tissues in the eye through topical administration. In addition, its lack of photosensitivity would allow for increased stability of a PRG-containing topical formulation.

Summary of previous findings and expansion: We prepared and characterized an extended action microemulsion (ME)-based formulation for delivery of PRG to the eye¹². Our ME was designed to overcome the drawbacks with traditional eye drops which comprise rapid drainage, short corneal contact time and minimal corneal penetration; all of which lead to reduced efficacy and poor patient adherence. We accomplished this by engineering a multilayered ME using highly biocompatible components with in situ gelling properties that improve bioadhesion, enhance corneal penetration and provide continuous pharmacological action for >30 hr. Because our ME has a small particle size (<20 nm), it is transparent and does not blur vision. We determined that there is a dose response reduction in IOP to increasing doses of PRG ME, with 0.6% PRG being the minimal dose that provides the maximum effect with a reduction in IOP of nearly 40% that does not return to baseline until 34 hours after dosing (Test 112). We also determined that repeated daily dosing with PRG ME for 21 days did not result in a decrease in efficacy (aka tachyphylaxis) of the formulation (Test 2¹²). Throughout the study, IOP remained at a reduced level in the treated eye, while the control eye remained at the elevated baseline IOP. We also determined the distribution of PRG in the eye and body after the conclusion of the tachyphylaxis study (Test 2¹²). After 21 consecutive days of topical dosing with our PRG ME, we determined that the drug reached target tissues in the eye. The control eye that received blank ME contained miniscule levels of PRG, as did plasma and all peripheral organs. Slit lamp and histopathological exams determined that the drug-loaded ME is safe and well tolerated by the eye¹².

Health Benefits: Given the rising prevalence of glaucoma and the predicted increase in human longevity, glaucoma incidence will increase accordingly. In 2013, the economic burden of glaucoma in the US was estimated at ˜$5.8 billion²⁹. The development of our product would provide a therapeutic approach for management of glaucoma that will have a market share among the glaucoma therapeutics. This stance is supported by PRG's new MOA, once daily dosing regimen with superior efficacy compared to market leaders (IOP lowered by ˜40% which does not return to baseline for >30 hrs), and high biocompatibility. Because PRG has a unique MOA, high efficacy and duration, without wishing to be bound by theory, it cannot only be a monotherapy, but also be used in combination with other glaucoma therapeutics. Without wishing to be bound by theory, our topical formulation will sustain a peak annual US market of $438 million based on prevalence estimates of 3 million affected Americans, a cost of $200/5 ml bottle, a 10% market share and both eyes of a patient will receive a single daily drop. With the increase in prevalence to 6.3 million Americans that is predicted by 2050³⁰, our US market share can increase to $920 million following the same assumptions. Although we are designing our formulation to be used with PRG, this ME can be modified to be suitable for incorporation of water-insoluble drugs. Therefore, the utility of our microemulsion reaches beyond our current drug (PRG) and our current target disease (glaucoma). Comparison of microemulsions with other nano-drug delivery systems (e.g. nanoparticles) illustrates that microemulsions are stable, easy to prepare, no energy requirement during preparation, have encapsulation efficiencies of 100% because there is no API loss during its preparation, and contain permeability enhancers that improve the corneal permeability of the incorporated drugs. These characteristics make microemulsions easy to scale up in batch size for pharmaceutical manufacturing companies to produce them for commercial use.

We have discovered several additional, unexpected findings. Our data show that CACNA2D1 is also expressed in retinal ganglion cell (RGC) bodies, as well as RGC axons in the optic nerve (ON). After 21 consecutive days of topical dosing with our ME, PRG reaches the tissues of the posterior eye, including the retina, at a concentration sufficient to inhibit CACNA2D1. Furthermore, topical dosing of our PRG ME mitigates ON damage in BXD29 mice, a pre-clinical model of normal tension glaucoma. Based on this evidence, we are pursuing this important lead and determining if CACNA2D1 can be a druggable target on RGCs to protect against glaucoma-induced RGC neurodegeneration, in addition to its IOP-lowering effects via the CB and outflow structures. Without wishing to be bound by theory, PRG formulation can deliver therapeutic dose to the retina and is neuroprotective to RGCs, without wishing to be bound by theory, our market share can increase, as it would be the only glaucoma therapy that can both lower IOP and offer direct neuroprotection to RGCs.

Approaches:

We have identified an IOP-modulating gene, Cacna2d1, as well as a drug—PRG—that binds to CACNA2D1 protein with high affinity and specificity to lower IOP^{15,11}. All current therapies have limitations including lack of sustained action, eye irritation, as well as other side effects and drug interactions. Changing therapies for patients who experience these side effects is challenging because all current therapies are linked to a restricted set of MOAs. Many patients require more than one drug to achieve target IOP levels. Both of these challenges are exacerbated by the limited availability of treatment options with different MOAs. PRG represents a new class of glaucoma drug that acts through a new, previously unknown target and MOA, thus providing a much-needed option for many patients. Because CACNA2D1 is localized to both inflow and outflow structures in the eye (FIG. 2)^{12,15}, PRG can offer dual, coordinated actions to lower IOP. PRG has been approved by the FDA and is currently a generic, which will allow us to repurpose it through the 505(b)(2) regulatory pathway and provide a much-needed new class of glaucoma therapies.

We have prepared and characterized an extended-release, multilayered, bio-adhesive, well-tolerated topical ME-based formulation for delivery of hydrophilic drugs to the eye. Our ME is designed to overcome the drawbacks with traditional eye drops that include rapid drainage, short corneal contact time and minimal corneal penetration, all of which lead to reduced efficacy and poor patient compliance because of the need for multiple daily dosing. The improved efficacy is supported by increased muco-adhesive properties, increased corneal permeability, the miniscule particle size, penetration enhancing ability and sustained release. We will improve the current 3-month stability of this formulation to lengthen the shelf-life for ease of clinical application.

In addition to the advantageous physical characteristics of ME, there are benefits that make ME production superior to other extended-release alternatives. MEs can readily be manufactured as sterile. Moreover, encapsulation efficiency is roughly 100% during ME production, thus allowing the process to be scaled up to make large batches. From an economic point of view, ME manufacturing is efficient because it does not require any energy input other than simple mixing.

Non-Limiting, Exemplary Approach

This will move our product toward regulatory approval for our extended-release PRG ME for lowering IOP in glaucoma and/or ocular hypertension patients. We established the minimal once daily dose that provides maximal IOP-lowering response in rabbits and demonstrated that single once daily dosing for 21 consecutive day) does not diminish the efficacy of our PRG ME, yet maintains its safety and biocompatibility with the eye and minimal systemic exposure. We also showed that topical dosing with our PRG ME increases the amount of drug that enters the eye. In addition we determined that PRG can reach the retina, thus allowing for this mode of dosing for posterior eye targets, while limiting the drug that reaches the un-dosed fellow eye or off-target organs¹². This study focuses on work to enhance the value propositions of our drug product by extending the formulation's current 3-month stability, while maintaining full efficacy making it a viable pharmaceutical product with a shelf-life of at least 12 months. We will scale-up our manufacturing to large scale batches and GLP manufacture for toxicology studies. We will perform toxicology studies. PRG is currently an FDA-approved drug used for other purposes. PRG has been an approved product for pain management since 2005 and has been prescribed to and taken by over 16 million people (Lyrica website FAQ). In addition, we have shown there is no evidence of systemic exposure after ophthalmic application of PRG¹².

Research Design and Methods

1: Increase Stability, while Maintaining Efficacy, of Our New Topically Applied Microemulsion Formulation.

Objectives: We will validate the formulation of a new glaucoma drug with sustained IOP lowering properties, and demonstrate that it is both physically and chemically stable for a minimum of 1 year and is efficacious in our rabbit model of glaucoma after extended period of storage to ensure potency with the improved formulation(s).

Increase the Chemical Stability of Our ME Formulation.

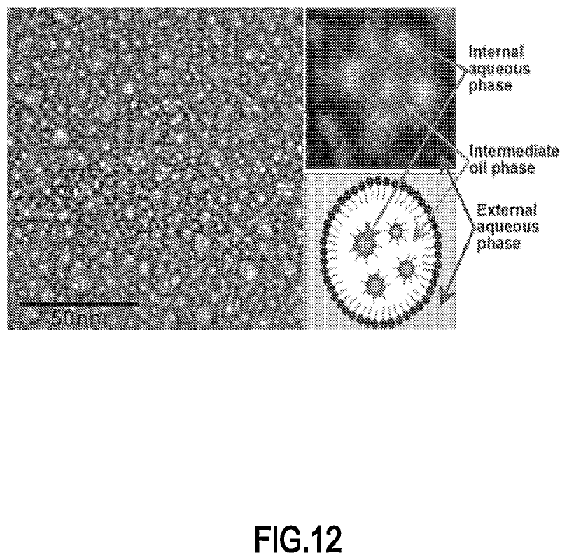

Results: Pregabalin (PRG; BCS class I), targets CACNA2D1 with high affinity and specificity¹¹. We determined that, when topically applied, a single drop of PRG aqueous solution lowered IOP in a dose dependent manner. However, the amplitude and duration of response were limited and unpractical for clinical use. To increase the efficacy of PRG as an IOP-lowering drug, we engineered a new microemulsion topical formulation¹². Our formulation was designed as a water-in-oil-in water microemulsion with 40% and 60% of the PRG in the innermost and outermost aqueous phases, respectively. Compartmentalization of PRG in two aqueous phases supports both a rapid onset and long duration of IOP-lowering action. Drug release from the outermost aqueous phase is fast, allowing for rapid IOP reduction. To be released from the innermost aqueous phase, however, PRG must pass through two interfaces—the inner water/oil interface and the outer oil/water interface—after which it must diffuse through the viscous polymer solution that constitutes the outmost aqueous phase of our multilayered ME (FIG. 12). Collectively the movement across both interfaces slows movement of PRG from our formulation and supports the extended IOP-lowering effect. After diffusion from the outermost polymer, PRG is available for absorption through the cornea. To ensure that the formulation will remain on the corneal surface for enough time to allow for the sustained release of the drug, the outermost hydrogel layer is prepared from Carbopol 981, which has excellent bio-adhesive properties.

TABLE 1
PRG ME droplets are <20 nm with a high zeta potential¹².

| Mean droplet | | | | Zeta | |
|---|---|---|---|---|---|
| size (nm) | | PDI | | potential (mV) | |
| Blank | Medicated | Blank | Medicated | Blank | Medicated |
| 16.0 ± 0.1 | 15.4 ± 0.0 | 0.26 ± 0.0 | 0.26 ± 0.0 | −30.1 ± 1.3 | −26.3 ± 0.6 |

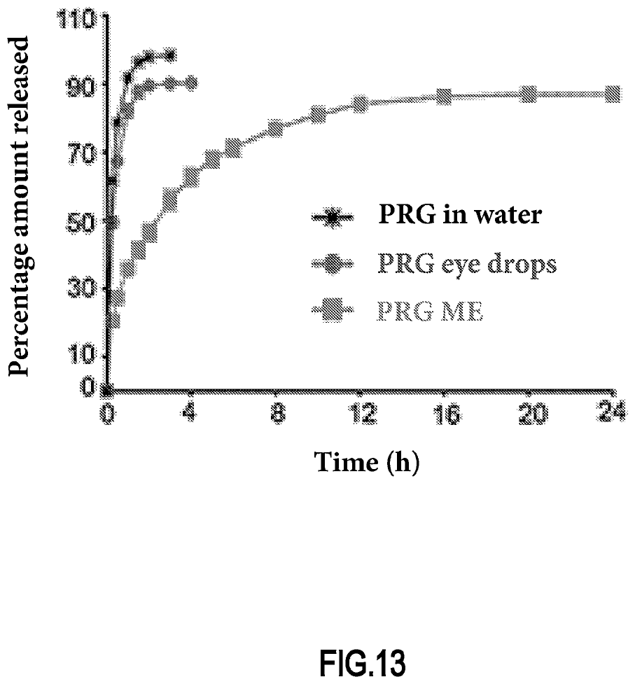

Our ME has a small droplet size, which is not affected by drug loading (Table 1). Its low PDI indicates that the variation in size is minimal, which is confirmed by transmission electron microscopy (TEM; globules FIG. 12). X-ray diffraction data demonstrate that PRG is soluble in our ME and no PRG crystals are present¹². Additional characterization revealed that compared to PRG eye drops, our PRG ME has lower viscosity, which facilitates its dispensing from the dropper, and higher bio-adhesion that will keep it localized within the eye after dosing¹². A feature is that incorporation of PRG into our ME prolongs its release time for more than 24 hours compared to 3 hr as an aqueous eye drop (FIG. 13). Given its high zeta potential (Table 1), ME can be stable. Following the guidelines put forth by the International Conference on Harmonization (ICH)³¹, we evaluated the chemical and physical stability of our formulation at 5°, 25°, 30°, and 40° C. Chemical stability was assessed by measuring the pH, size, PDI, zeta potential, drug release and drug content³¹. Physical stability was gauged using repeated freeze-thaw cycles and by ultracentrifugation^{32,33}. Our results demonstrate that the drug content of our ME, an indicator of our formulations' chemical stability, remained within the acceptable pharmacopeial limit for 3 months at 5° & 25° C., and for 2 months at 300 & 40° C. (Table 2). Although the physical characteristics our formulation did not vary and other indicators of chemical stability (pH, droplet size and charge) were unchanged during one year of storage, the retention of drug content has to be optimized to ensure successful development of a viable commercial drug product with a reasonable/practicable shelf-life for clinical trials and later use.

TABLE 2
Drug content of PRG (%) after storage for up to 4 months.
Our current PRG ME is chemically stable for 3 months at
5° & 25° C., and for 2 months at 30° & 40° C.
Other parameters were stable for at least 12 months.

| | Storage | Temperature (*C.) | | | |
|---|---|---|---|---|---|
| Parameters | months | 5 | 25 | 30 | 40 |
| pH | Initial | 5.32 ± | 5.32 ± | 5.32 ± | 5.32 ± |
| | | 0.12 | 0.12 | 0.12 | 0.12 |
| | 1^{st} | 5.40 ± | 5.41 ± | 5.31 ± | 5.37 ± |
| | | 0.04 | 0.02 | 0.02 | 0.03 |
| | 2^{nd} | 5.42 ± | 5.21 ± | 5.33 ± | 5.16 ± |
| | | 0.13 | 0.10 | 0.04 | 0.02 |
| | 3^{rd} | 5.39 ± | 5.31 ± | 5.30 ± | 5.10 ± |
| | | 0.09 | 0.08 | 0.04 | 0.04 |
| | 4^{th} | 5.39 ± | 5.18 ± | 5.15 ± | 4.84 ± |
| | | 0.09 | 0.04 | 0.03 | 0.03 |
| Droplet | Initial | 15.58 ± | 15.58 ± | 15.58 ± | 15.58 ± |
| size | | 0.09 | 0.09 | 0.09 | 0.09 |
| (nm) | 1^{st} | 15.52 ± | 15.64 ± | 15.25 ± | 15.31 ± |
| | | 0.07 | 0.27 | 0.17 | 0.07 |
| | 2^{nd} | 16.08 ± | 16.45 ± | 16.51 ± | 17.14 ± |
| | | 0.64 | 0.11 | 0.39 | 0.82 |
| | 3^{rd} | 15.95 ± | 15.94 ± | 16.57 ± | 16.81 ± |
| | | 0.11 | 0.27 | 0.73 | 0.16 |
| | 4^{th} | 16.39 ± | 16.12 ± | 16.05 ± | 16.31 ± |
| | | 0.45 | 0.19 | 0.31 | 0.18 |
| PDI | Initial | 0.26 ± | 0.26 ± | 0.26 ± | 0.26 ± |
| | | 0.01 | 0.01 | 0.01 | 0.01 |
| | 1^{st} | 0.25 ± | 0.26 ± | 0.25 ± | 0.25 ± |
| | | 0.00 | 0.01 | 0.00 | 0.00 |
| | 2^{nd} | 0.26 ± | 0.26 ± | 0.26 ± | 0.29 ± |
| | | 0.02 | 0.01 | 0.00 | 0.06 |
| | 3^{rd} | 0.26 ± | 0.26 ± | 0.29 ± | 0.26 ± |
| | | 0.00 | 0.01 | 0.05 | 0.00 |
| | 4^{th} | 0.26 ± | 0.26 ± | 0.26 ± | 0.26 ± |
| | | 0.00 | 0.00 | 0.00 | 0.01 |
| Zeta | Initial | −31.42 ± | −31.42 ± | −31.42 ± | −31.42 ± |
| potential | | 1.14 | 1.14 | 1.14 | 1.14 |
| (mV) | 1^{st} | −39.27 ± | −36.89 ± | −37.20 ± | −38.90 ± |
| | | 7.90 | 1.43 | 3.46 | 1.31 |
| | 2^{nd} | −31.77 ± | −35.41 ± | −41.99 ± | −47.38 ± |
| | | 2.49 | 2.44 | 0.90 | 1.74 |
| | 3^{rd} | −35.88 ± | −41.39 ± | −41.52 ± | −48.36 ± |
| | | 2.09 | 1.35 | 3.78 | 0.40 |
| | 4^{th} | −30.02 ± | −30.40 ± | −32.46 ± | −34.12 ± |
| | | 1.83 | 0.32 | 0.84 | 0.90 |
| Drug | Initial | 100.00 ± | 100.00 | 100.00 ± | 100.00 ± |
| content | | 0.00 | 0.00 | 0.00 | 0.00 |
| (%) | 1^{st} | 100.00 ± | 100.00 ± | 100.00 ± | 100.00 ± |
| | | 1.23 | 0.88 | 1.98 | 4.17 |
| | 2^{nd} | 104.09 ± | 103.48 ± | 100.55 ± | 100.31 ± |
| | | 0.74 | 1.10 | 1.09 | 1.70 |
| | 3^{rd} | 96.76 ± | 92.21 ± | 86.25 ± | 82.54 ± |
| | | 2.90 | 1.20 | 0.70 | 7.28 |
| | 4^{th} | 84.61 ± | 85.56 ± | 81.54 ± | 69.24 ± |
| | | 1.54 | 1.53 | 1.67 | 2.63 |

Non-Limiting Research Design and Methods: We will optimize our formulation to improve the chemical stability of PRG in our ME. This will be done in two steps: 1) short-term stability and antioxidant screening studies, followed by; 2) a stability study following the ICH guidance. Without wishing to be bound by theory, we will have a formulation that has a minimal shelf-life of about 12 months.

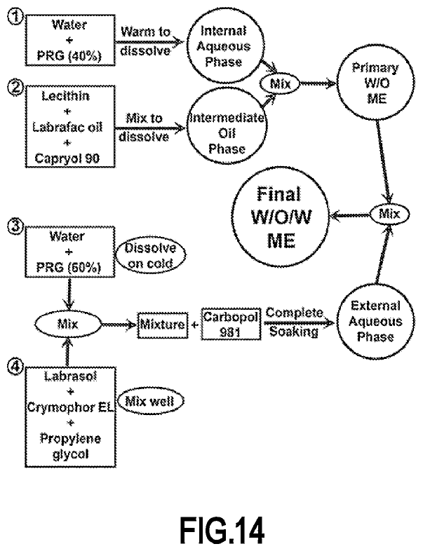

Preparation of the microemulsion: Preparation of our multilayered water-in-oil-in-water (w/o/w) ME bio-adhesive eye drops is achieved in several steps. The primary w/o ME is prepared and further emulsified into the external aqueous solution in which the bio-adhesive polymer has been previously soaked. To determine the appropriate ratios of the primary microemulsion components, several triphase diagrams were constructed by water titration method using different hydrophobic surfactants¹². The primary w/o ME that we selected consists of 20% water (in which 40% of the drug is dissolved; FIG. 14, step1)+[30% oil (labrafac lipophile WL 1349)+50% surfactant mixture (capryol 90 & soybean lecithin, 1:1; FIG. 14, step 2)]. The external aqueous phase consists of 50% water in which 60% of the drug is dissolved (FIG. 14, step 3)+a mixture of (10% labrasol+10% cremophor EL+30% propylene glycol) (FIG. 14, step 4). The bio-adhesive polymer (Carbopol 981, 0.15%) will be soaked in the previously prepared external aqueous phase and allowed to swell overnight to produce a viscous polymer/surfactants solution. The prepared w/o microemulsion that contains 40% of the polymer/surfactant solution and mixed well until the clear multilayered w/o/w microemulsion bioadhesive eye drop forms.

**Addition of antioxidants: During storage, degradation of active pharmaceutical ingredients (APIs) in formulations occurs through several pathways including acidic and/or basic degradation, oxidative degradation, aqueous hydrolysis, photolysis and thermal degradation. PRG is stable against aqueous hydrolysis, photolysis and thermal degradation, and relatively stable against acidic hydrolysis³⁴. In contrast, it is unstable under basic and oxidative conditions¹⁴. Based on these data, upon storage in an aqueous medium, PRG can be susceptible to degradation at either an alkaline pH or by oxidative degradation. Our ME has a weak acidic pH (5.4±0.05), therefore the factor that is negatively affecting the chemical stability of PRG in our ME is oxidative degradation.

TABLE 3
Strategy for testing aqueous- and lipid-soluble antioxidants to improve
ME stability.

| | | Water soluble antioxidants | | | No water |
|---|---|---|---|---|---|
| | | | | Sod. | soluble |
| | | Glutathione | TPGS | Metabisulfite | anti- |
| | | (0.2%) | (1%) | (1%) | oxidant |
| Oil soluble | Alpha | Formula #1 | Formula | Formula | Formula |
| antioxidants | tocopherol | | #2 | #3 | #4 |
| | (1%) | | | | |
| | BHT | Formula #5 | Formula | Formula | Formula |
| | (0.02%) | | #6 | #7 | #8 |
| | BHA | Formula #9 | Formula | Formula | Formula |
| | (0.02%) | | #10 | #11 | #12 |
| | BHT + | Formula | Formula | Formula | Formula |
| | BHA | #13 | #14 | #15 | #16 |
| | (1:1; | | | | |
| | 0.02%) | | | | |

| | | | | |
|---|---|---|---|---|
| No oil soluble | Formula | Formula | Formula | — |
| antioxidant | #17 | #18 | #19 | |

Note:
Due to its emulsifying properties, in formulations containing TPGS, the ratio of formulation components will need to be determined using our published methods¹².

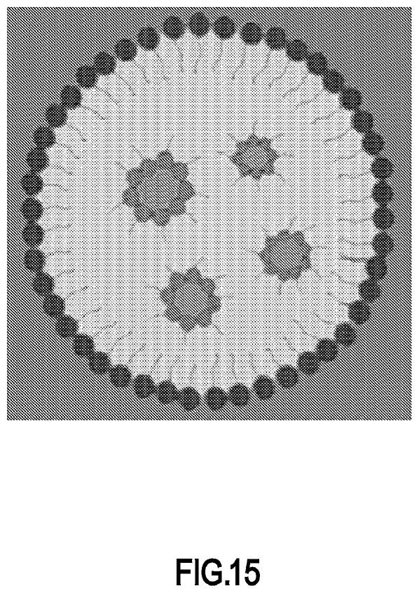

To combat oxidative degradation, and thereby improve the chemical stability of our PRG ME, we will incorporate one or more water-soluble antioxidants to the internal and external aqueous phases (FIG. 15, blue), and/or oil-soluble antioxidants to the intermediate oil phase of our ME (FIG. 15, tan) to protect PRG from oxidative degradation during storage. Antioxidants are included in pharmaceutical preparations to enhance the stability of the API that is susceptible to chemical degradation by oxidation. Water-soluble antioxidants to be tested in our ME include: glutathione (CAS #70-18-8); tocopheryl polyethylene glycol succinate (TPGS; CAS #9002-96-4); or sodium metabisulfite (CAS #7681-57-4). Oil-soluble antioxidants to be tested include: alpha tocopherol (vitamin E; CAS #10191-41-0); butylated hydroxytoluene (BHT; CAS #128-37-0); or butylated hydroxyanisole (BHA; CAS #25013-16-5). To determine the most effective antioxidant or antioxidant combinations, a pre-stability and antioxidant screening study will first be conducted, followed by a full stability study following ICH guidance. Many of these ingredients have been used to prolong stability of many APIs and can reach a stability of 12 months.

Evaluation of short-term formulation stability during antioxidant screening: Screening of antioxidants will be achieved by adding one or more antioxidants into our ME individually or in combination, as indicated in Table 3. The formulations containing antioxidants will be subjected to accelerated stability studies by storing them at three different temperatures 25°, 30°, 40° C. for three months. The stored formulations will be evaluated every 15 days regarding their physical appearance, drug content and pH. If all formulations remain stable for three months, this short-term study will be extended for six months. Depending on these accelerated stability results, the most effective antioxidant(s) will be selected for use in the final optimized formulation.

Full stability studies of the optimized formulation following the ICH guidance: Depending on the results of the short-term stability and antioxidant screening studies, the final optimized formulation containing the most effective antioxidants, as well as the original formulation without antioxidants will be subjected to a stability study according to the ICH guidelines as follows: The formulation will be evaluated regarding physical stability (freeze-thaw cycles and the centrifugation test) and chemical stability following ICH guidelines. Because we do not know the best storage conditions in which we intend to store our formulation during its shelf life, we will test different conditions the including room temperature, refrigerator or freezer conditions. For long-term stability studies, the formulation will be stored at −20° C., 5° C. and 25° C. for 1 year. An intermediate stability study will be conducted at 30° C. for 6 months. An accelerated stability study will also be conducted at 40° C. for 6 months using our standard methods. During storage the formulations will be evaluated monthly regarding their drug content, pH, droplet size, PDI and zeta potential. The release pattern will be also tested initially, then every 6 months to evaluate the ability of the ME to maintain its sustained release behavior.

The following methods will be used to evaluate physical and chemical stability (triplicate samples):

Physical stability: Physical stability will be gauged using repeated freeze-thaw cycles and by ultracentrifugation^{32,33}.

PRG content: Our HPLC-UV method¹² will be used to determine the drug content in the formulations and release samples.

pH determination: The pH will be measured using a pH meter (Corning pH meter 440) 12.

Average droplet size, polydispersity index (PDI) and zeta potential measurement: The average droplet size, PDI and zeta potential of our ME formulations will be determined after suitable dilution using a Zetasizer (Nanoseries, nano-ZS)³⁵. All measurements will be performed at 25° C.

Test 1.2: Confirmation of efficacy of optimized formulation from Test 1.1 in a rabbit model of elevated IOP.

TABLE 4
Pharmacodynamic parameters of formulations
shown in FIG. 6

| | Ophthalmic Eye Drops | | |
|---|---|---|---|
| | PRG | PRG in | PRG |
| Pharmacodynamic | Carbopol 981 | Carbopol | W/O |
| parameters | ME | 981 | ME |
| Baseline IOP | 21.4 ± 0.7 | 22.1 ± 0.4 | 16.7 ± 1.2 |
| IOP at Tₘₐₓ | 12.3 ± 0.5 | 15.7 ± 0.4 | 12.7 ± 0.7 |
| ΔIOP | −9.1 ± 0.8 | −6.5 ± 0.4 | −4.0 ± 0.58 |
| % Reduction | 42.3 ± 2.6 | 29.4 ± 1.4 | 23.8 ± 1.9 |
| in IOP | | | |
| Tₘₐₓ (h) | 3.3 ± 0.9 | 3.3 ± 1.5 | 2.33 ± 0.33 |
| T_{end} (h) | 32.7 ± 1.3 | 9.3 ± 0.7 | 7.33 ± 1.33 |
| AUC (%, H) | 788.6 ± 36.8 | 172.3 ± 16.9 | 98.9 ± 13.8 |

Data are expressed as mean ± SEM;
n = 3
Tₘₐₓ: time to maximize response in hours
T_{end} (h): time to end of response in hours
AUC (%, H): total area under % IOP reduction versus time curve

Data: We evaluated the efficacy of our PRG ME in Dutch belted rabbits¹² and demonstrated that a single drop of our ME formulation maintained a reduced IOP (42.3% reduction; Table 4) that returned to baseline at 34 h after single application (AUC=788.6%·hr). In the absence of our ME, the same drug in Carbopol 981 gel produced only a 29.4% IOP reduction that returned to baseline at 10 h (AUC=172.3%·hr), demonstrating that incorporation of the drug in our ME enhanced its efficacy. Moreover, the combined effect of all three layers of our W/O/W ME is critical for enhancing the efficacy of PRG. We have also characterized the ME and determined that it increases penetration of PRG across the cornea and is safe and well tolerated in the eye¹². Using this same model, we will evaluate our optimized formulations from Test 1.1.

To evaluate long-term use of our PRG ME, we performed a tachyphylaxis, biocompatibility and biodistribution study using daily dosing for 21 consecutive days¹². Our data demonstrate that after the first dose of PRG ME, IOP was maintained at a normal physiological range of 13-14 mmHg; p<0.001 compared to baseline of 19.3 mmHg). Importantly, after 21 days of dosing, the cornea and conjunctiva of the rabbits appear healthy with no cells or flare in the anterior chamber, providing evidence that our ME is well tolerated in the eye. This finding is also confirmed with histopathology¹².

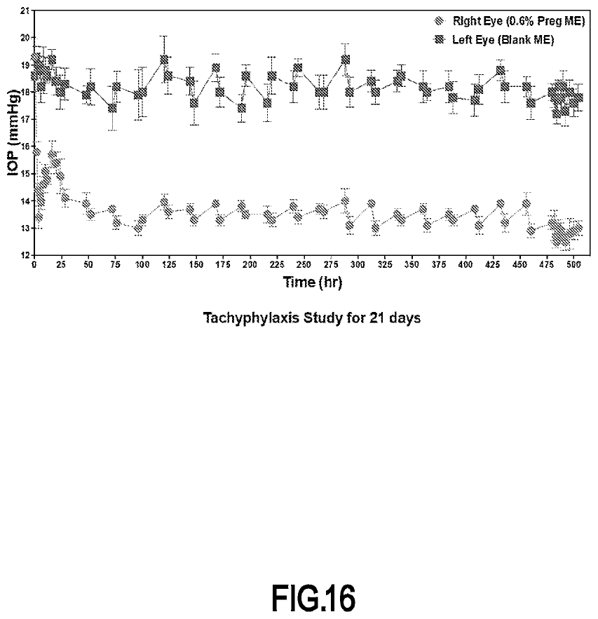

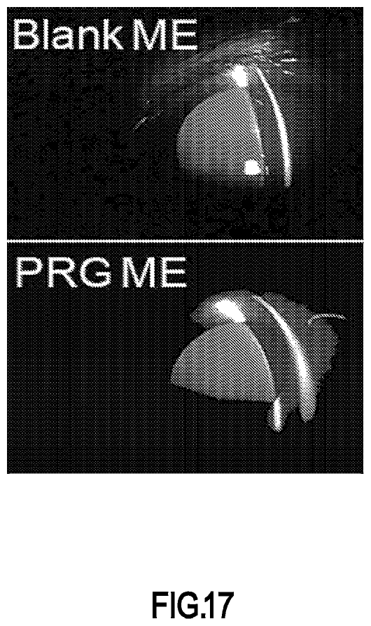

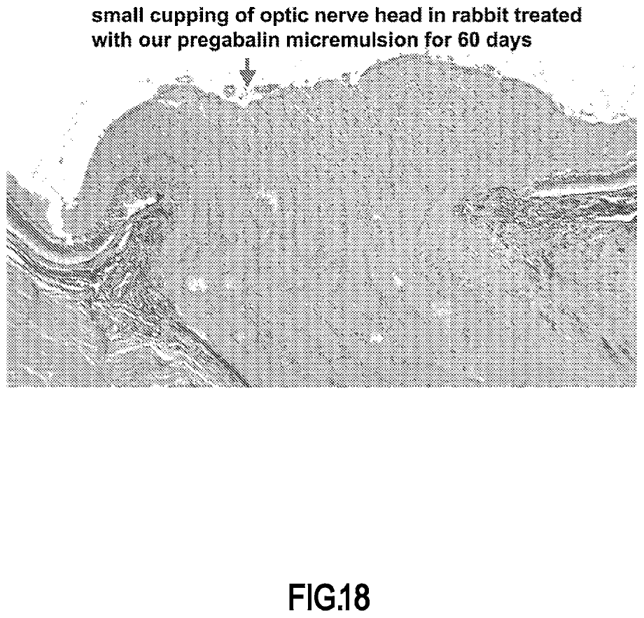

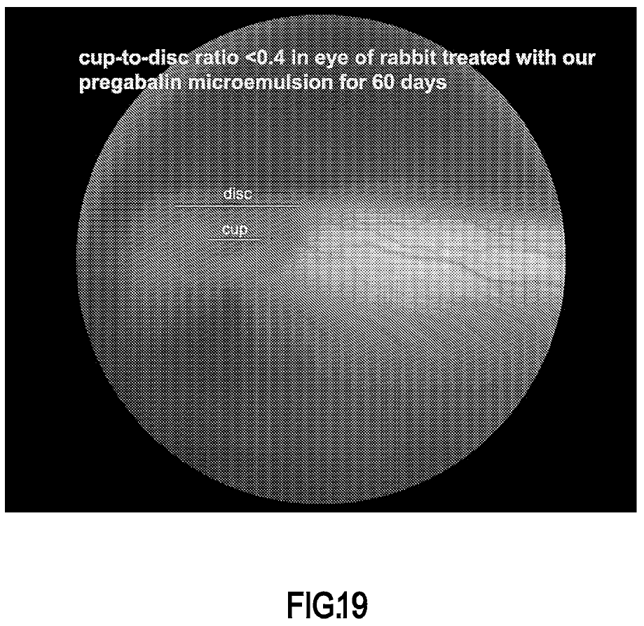

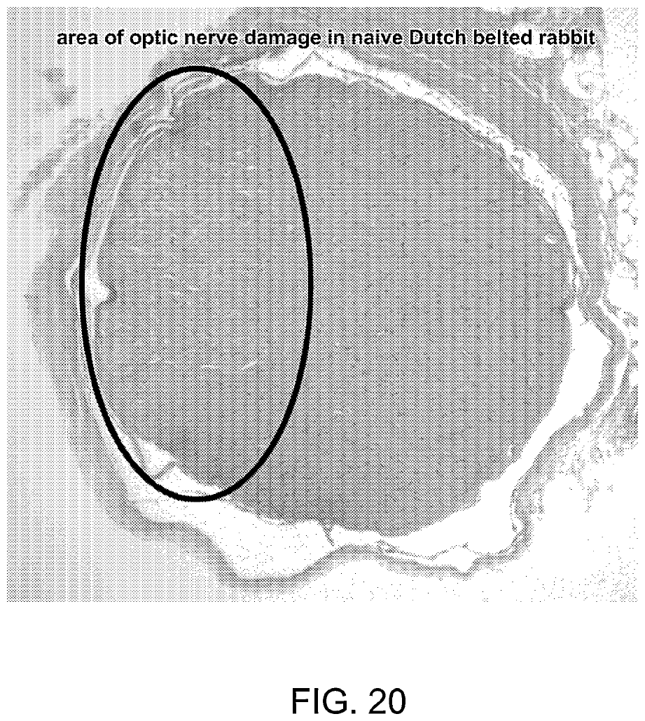

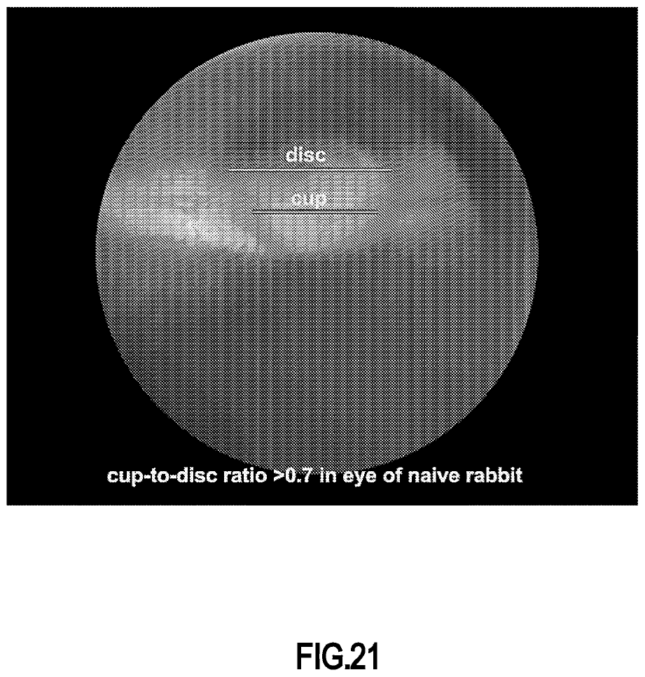

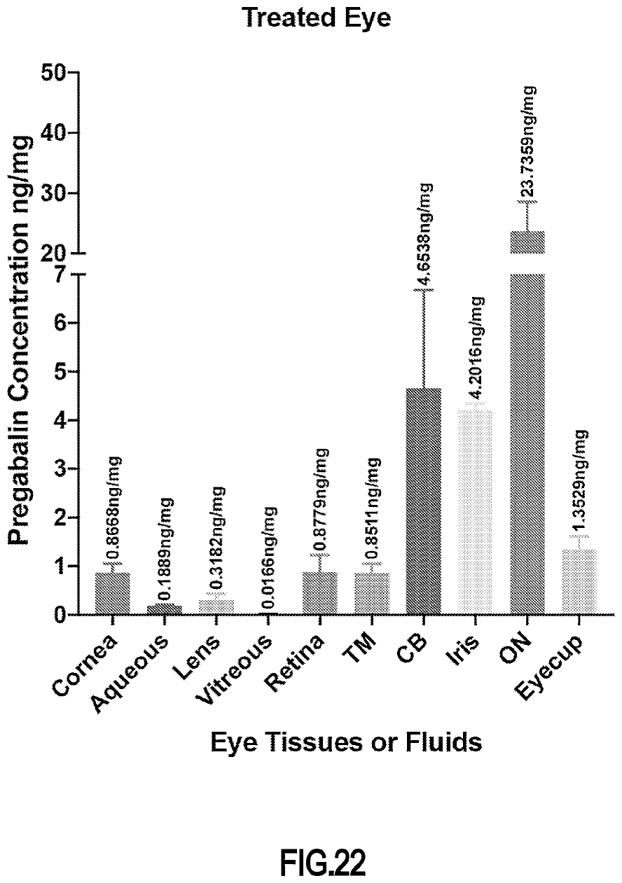

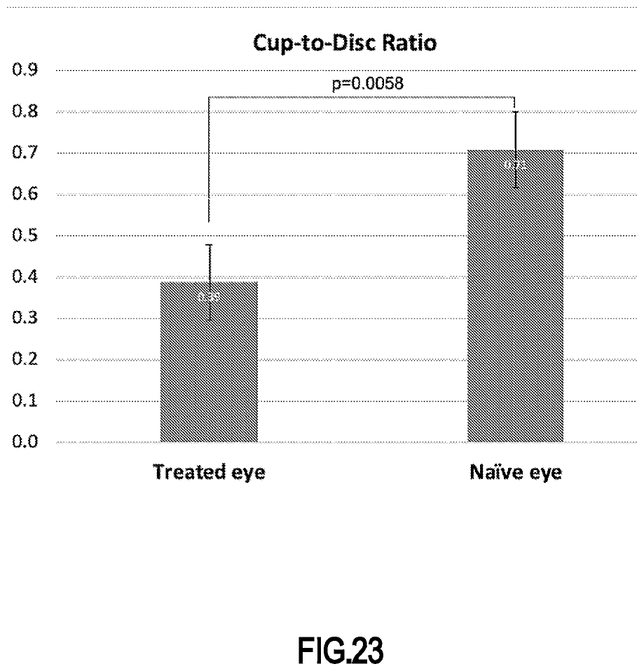

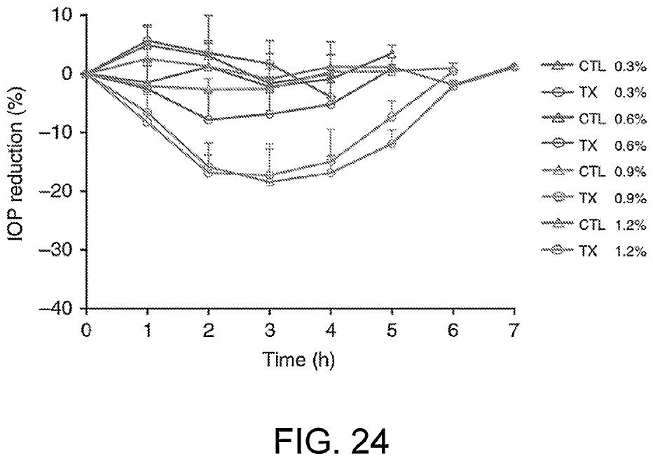

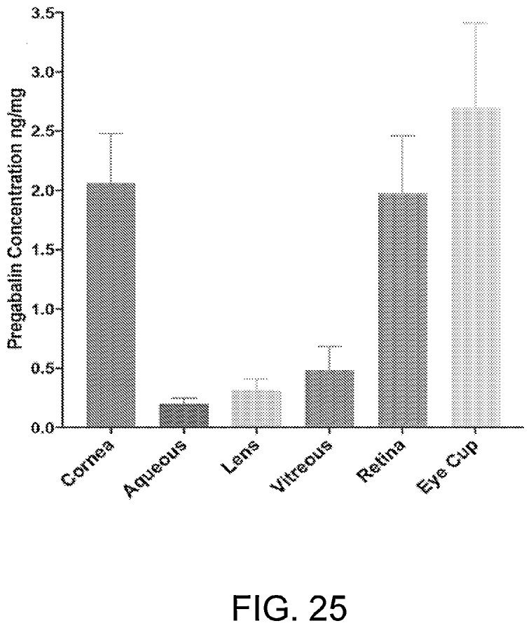

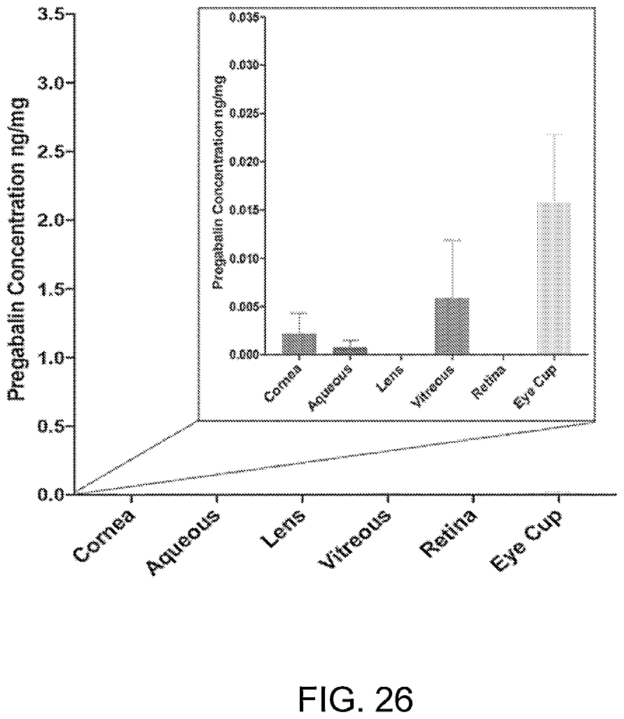

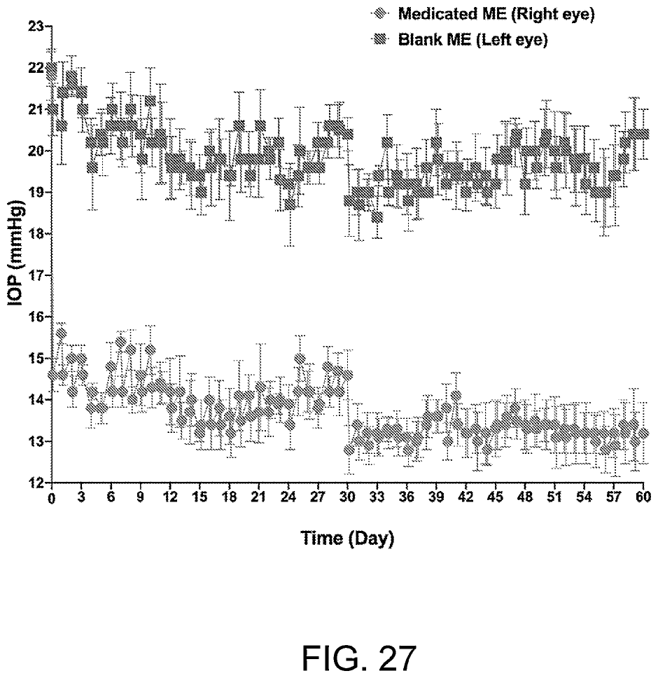

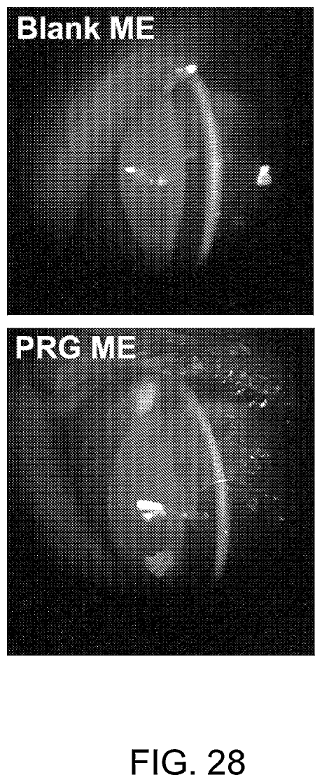

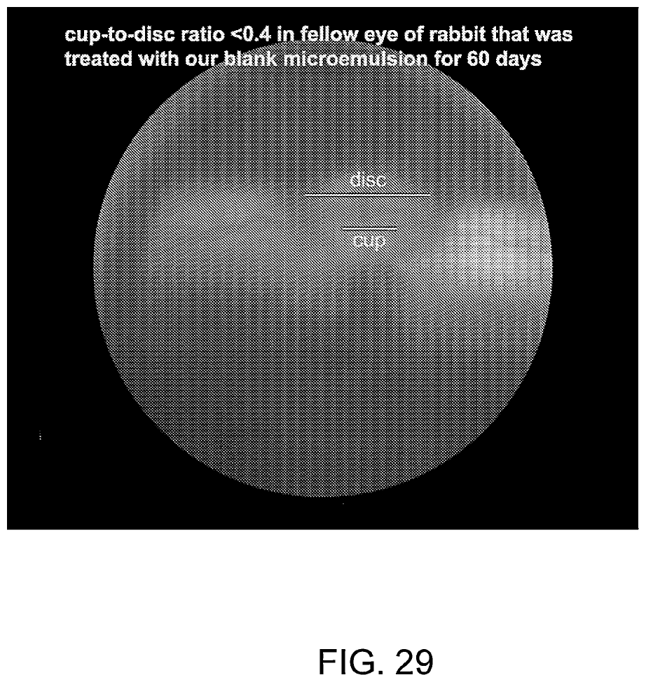

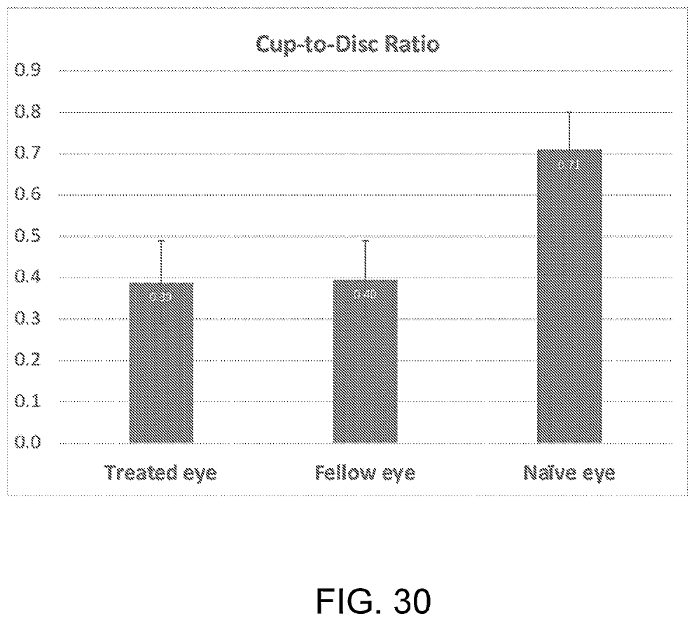

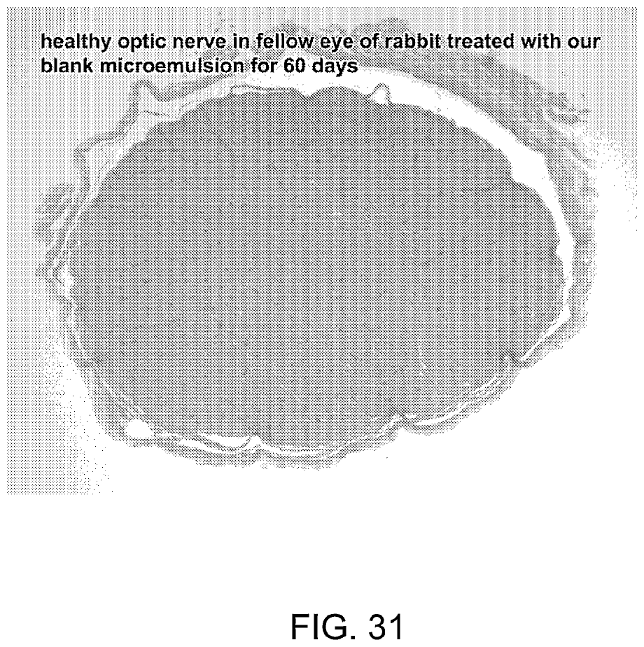

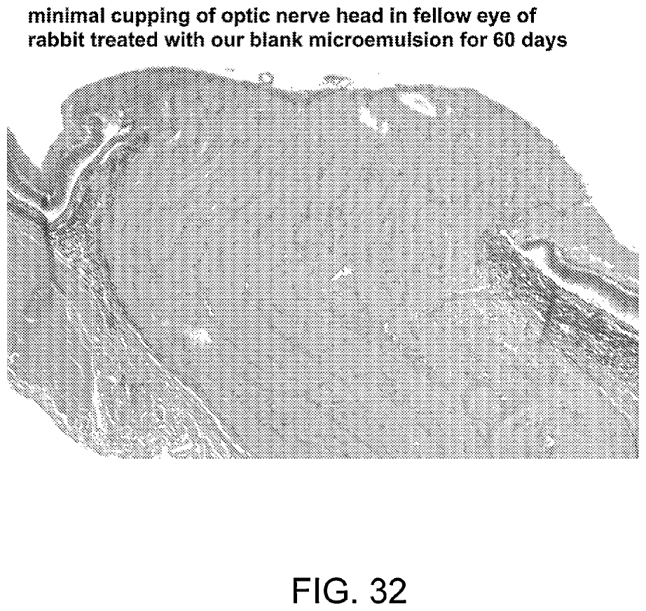

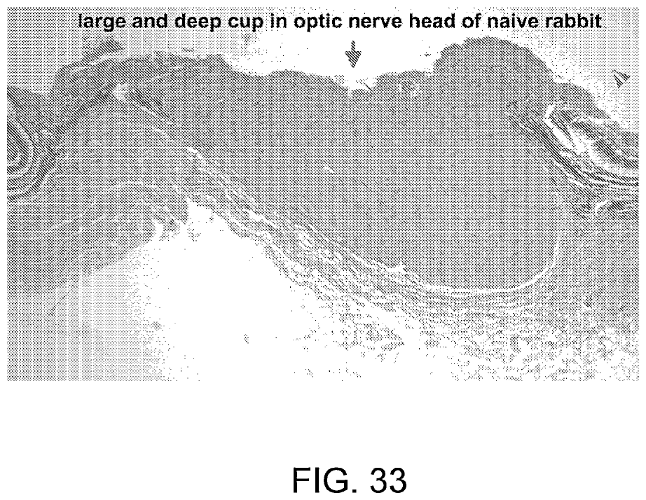

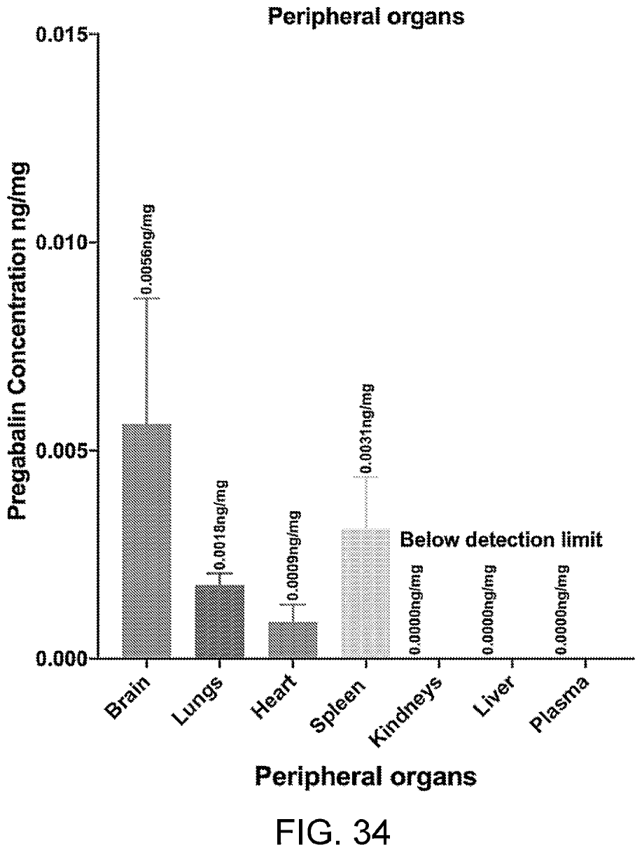

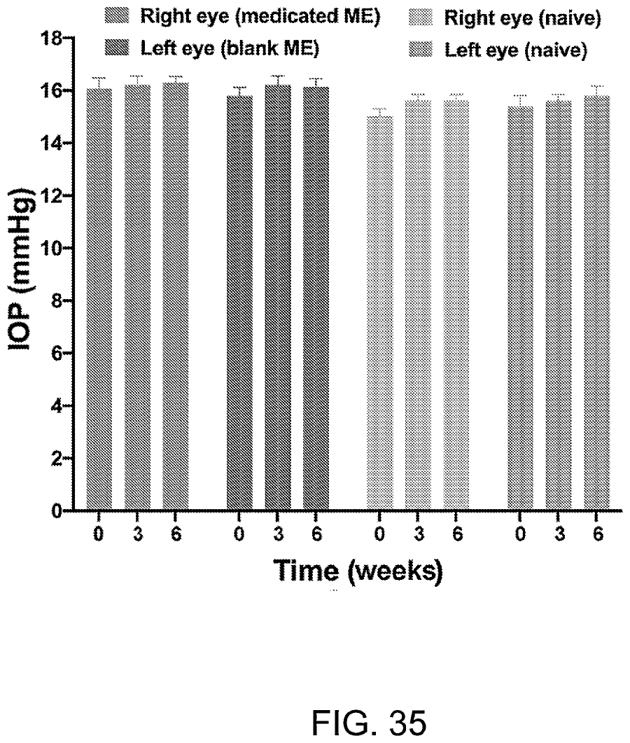

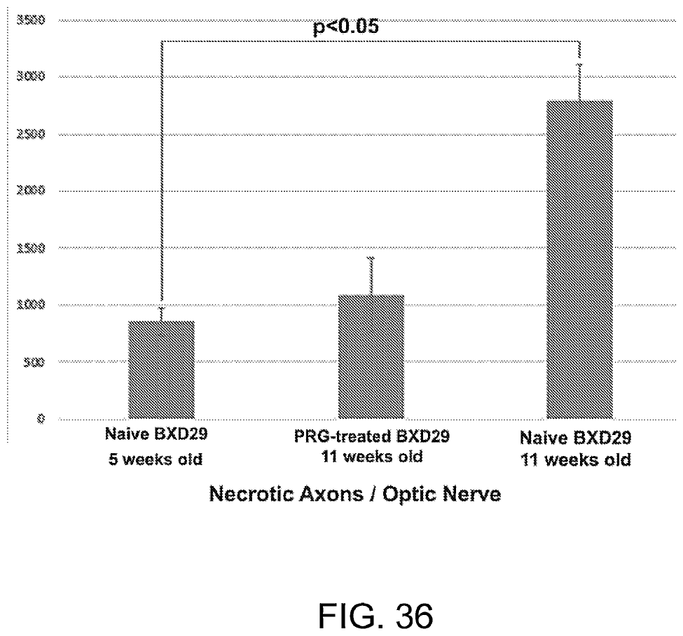

Research Design and Methods: Dutch belted rabbits (Envigo Rabbitry; formerly Covance) will be used in this single-dose design study. Rabbits from this rabbitry can have higher baseline IOP values than those purchased from other rabbitries. Rabbits (equally balanced between males and females) will be acclimated to handling and IOP measurements for at least seven days before initiation of the study. Slit-lamp biomicroscopic and fundoscopic examinations will be performed before and after the study. Our top three formulations from Test 1.1 will be tested in this model using our methods¹². The IOP of both eyes will be measured using a rebound tonometer (Tono-Pen AVIA Vet, Reichert) immediately before the formulation application (baseline, 8 am) and at hourly time intervals after application until the IOP returns to baseline. Using a sterile micropipette tip, thirty microliters of the formulation containing PRG ME or blank control will be instilled topically into one eye of each rabbit, while the fellow eye will receive the formulation vehicle, and therefore serve as controls. Rabbits will be dosed daily at 8 am for 21 days, and IOP collected at 8 am (immediately pre-dosing) and at the time at which the maximum IOP reduction is measured (Tₘₐₓ) using our methods¹². Curves similar to FIG. 16 will be generated. Biocompatibility of the antioxidant-containing formulations will be assessed using with slit lamp biomicroscopy as in FIG. 17. The person performing the studies will be blinded regarding which eye receives the medicated formation and which one receives the blank control for each rabbit. Assuming a power of 80%, and a marginal difference in IOP outcome of 30%, we estimate that this study will require 10 rabbits. We will also evaluate these formulations in C57Bl/6J (B6) mice and cynomolgus monkeys, which will allow for species confirmation and comparison.

Without wishing to be bound by theory the addition of one or more antioxidants to the water and/or oil phases of our ME will improve the chemical stability of PRG and prevent its degradation during storage. MEs are stable formulations and can retain their physical stability as long as its water/oil ratio remain constant. Because antioxidants will be added as a part of the oil and/or the water contents of the ME, the phase volume ratio will remain constant, which can maintain the physical stability and integrity of our MW. The optimized ME with added antioxidants will continue to be highly efficacious with minimal to no changes in IOP-lowering capacity or duration of action. The chemical stability of PRG in an oral solution has a stability of >1 year (NDA 22-488).

Alternative approaches: While the short-term stability and antioxidant screening study will determine if antioxidants improve the chemical stability of our ME, antioxidants alone may not be sufficient to extend the shelf life of our product to two years. Including the evaluation of cooler storage temperatures (5° & −20° C.) will inform us if cold storage is necessary to improve the formulation stability. If the incorporation of antioxidants into our ME or changing the storage temperatures fail to increase its stability, we will explore the incorporation of antioxidants (e.g. BHT, BHA or a combination of them in 1:1 ratio) in the bottle material (HDPE) as well as packaging under nitrogen atmosphere. Because most of the oxidation reaction happens at the interface between the bottle wall and the formulation, rather than within the bulk of the formulation, the incorporation of antioxidant in the bottle material and packaging under nitrogen gas will help to cease the degradation of PRG due to oxidation.

Without wishing to be bound by theory, Test 1 can demonstrate the increased stability of our ME formulation to one year after the addition of antioxidants to maintain efficacy. The results from the accelerated stability will be used to calculate trend analysis plots for each formulation. We will use these plots to inform our formulation transfer to our GMP manufacturer (Test 2).

2: Manufacture of Drug Product Under Good Laboratory Practice (GLP) Conditions for Toxicology Studies.

Objectives: We will optimize and qualify the test methods to analytically assess our API starting material and manufactured drug product. We will manufacture GLP drug product and begin to analyze the impurities under ICH guidelines. This can provide: 1) drug product preparation method transfer to manufacturing site; 2) production of an engineering scale-up GLP batch that will be suitable for toxicology studies; 3) identification and characterization of significant impurities and degradants (>0.10%). Our target is successful manufacture of GLP drug product for use in toxicology studies that is stable for a minimum of 1 year.

2.1: Optimization, Quantification and Transfer of Analytical Test Methods to Support Drug Testing During Manufacture, Quality Release, and Stability Studies.

HPLC-UV: A LC-UV analytical assay using reverse phase HPLC and ultraviolet (UV) detection was successfully developed for the estimation of PRG potency and purity¹². This method is used for both assessing the pregabalin API and also the final drug product.

Average droplet size, polydispersity index (PDI) and zeta potential measurement: The average droplet size, PDI and zeta potential of our ME formulations will be determined after suitable dilution using Zetasizer (Nanoseries, nano-ZS)³⁵. Measurements will be performed in triplicate at 25° C.

Transmission electron microscopy: The morphology of our PRG ME as well as droplet size confirmation will be done using transmission electron microscopy (TEM) (JEOL JEM1200EX II electron microscope). Briefly, the ME formulation was diluted 1:100 with MilliQ water. Two microliters of the diluted ME will be placed on 400 mesh copper grids covered with Formvar film (Electron Microscopy Sciences EMS). The grids will be allowed to dry for 2 h in a desiccator followed by negative staining with Uranyless EM stain (Electron Microscopy Sciences EMS) before examination by a TEM.

Determination of the viscosity: A cone (1.5°) and plate rotary viscometer (Brookfield DV-II+ programmable viscometer; Brookfield Engineering Laboratories) will be used to determine the viscosity of our formulations according to our protocol^{36,37}. Each formulation (500 μl) will be placed on the stationary plate of the viscometer for 5 min before each measurement to reach the running temperature. The viscosity will be measured in triplicate at 350.5° C.

Measurement of the bioadhesive force: The bioadhesive force of our ME eye drops will be determined by a simple method that depends on evaluation of the rheological synergism that happen upon mixing the bioadhesive polymer with mucin dispersions^{38,39}. Gastric mucin type II (15%, w/v) will be dispersed in simulated tear fluid (pH 7.4) and allowed to dissolve overnight at 4° C. Before measurement, the mucin dispersion was warmed to 35° C. and then mixed with the formulations that had been previously warmed to the same temperature. The viscosities of the mucin dispersion, formulations, and their mixture will be measured in triplicate using the Brookfield viscometer. Viscosity changes, due to bioadhesion as well as the bioadhesive forces, will be calculated using our method 1.

Research Design and Methods:

2.2: GLP Drug Product Manufacture and Stability for Toxicology Studies. To be Performed by IRISYS

Data: We have made small-scale batches of PRG ME in-house up to 500 g each using the method shown in FIG. 16. Our PRG ME is chemically stable for 3 months at 5° & 25° C., and for 2 months at 30° & 40° C. (Table 2), which we will further optimize to increase the chemical stability to 1 year in Test 1.1. The final optimized formulation will be transferred to IRISYS for GLP manufacture and assessment in preparation for future toxicology studies.

Batch Records will be generated for GLP demonstration batches of 5,000 bottles each for the top three formulations with the longest chemical stability from Test 1.1. These demonstration batches will be produced to establish the process and the sensitivity of the analytical test methods. The impurity profile in these demonstration batches, and the future GMP batch, can be similar based on the nearly identical processes followed. The demonstration batch will be used in the GLP toxicology studies. In addition, these engineering batches will be entered into stability testing for a minimum of 12 months at −20° C., 2-8° C., 25° C., and 40° C. under ICH guidelines. Because we plan to use this batch for all our toxicology studies, we will fill enough samples to generate results for 24, 30, and 36 months to allow us to track stability throughout our full toxicology program. However only samples aged through 12 months will be analyzed.

2.3: Exploratory identification and characterization of impurities found in PRG ME drug product.

Impurities and degradants (>0.10%) can be identified and characterized. Impurities within the PRG ME drug product can be: 1) known impurities present in the pregabalin drug substance; and 2) unknown impurities within our PRG ME formulation. The six known impurities and degradation products found in pregabalin³⁴ and potential sources are:
Alkene impurity, 4-ene impurity, or 4,5 dehydropregabalin (CAS #216576-74-8; Toronto Research Chemicals)
Diacid impurity, 3-Carboxy-5-methylhexanoic acid (CAS #5702-99-8; Toronto Research Chemicals or Simon Pharma Limited)
3-(2-amino-2-oxoethyl)-5-methylhexanoic acid (CAS #181289-33-8; Toronto Research Chemicals)
(S)-4-isobutylpyrrolidin-2-one, lactam impurity, or PD 0147804 (CAS #61312-87-6; Toronto Research Chemicals)
Dione impurity or 4-isobutylpiperidine-2-6-Dione (CAS #916982-10-0; Toronto Research Chemicals or Sigma Aldridge)
R-enantiomer impurity or PD 0144550 (CAS #148553-51-9; Toronto Research Chemicals or Sigma Aldridge)

Non-limiting, Exemplary Research Design and Methods: Exploratory impurity profiling of the initial GLP lot of our optimized PRG ME containing antioxidants will be performed using HPLC analysis of the drug product. Initial structural characterization of impurities with peak areas >0.10% will be performed by MS/MS analysis. To confirm these structures, the identified impurities will purchased and characterized using LC/MS, MS/MS, and NMR spectral data. Many of the impurities will be the same as is seen in current commercial PRG drug product (NDA 22-488) as these will be carried through from the drug substance.

Throughout the studies performed to the following can be carried out: 1) transfer and qualify necessary test methods for drug product release and stability; 2) manufacture a GLP batch for each of our top 3 formulations of high-quality drug product suitable for use in toxicology studies; 3) manufacture a GLP-batch of placebo/ME vehicle; 4) determine that this drug product stable at 2-8° C. and RT for a minimum of two years; 5) identify and characterize, as mandated by ICH guidelines, PRG impurities that have been identified by analyses.

Metrics of Test 2 can comprise: 1) manufacture of GLP drug product for use in toxicology studies; 2) 6-month accelerated stability data; and 3) 12-month long term stability data under ICH guidelines.

3: Toxicology Assessment.

3 can determine the no-observed-adverse-event-level (NOAEL) of our PRG ME in rabbits (Test 3.1), which will guide the selection of appropriate doses of future GLP long-term repeat dose studies in rabbits.

3.1: Non-GLP Four-Week, Repeat Dose, Dose Range-Finding Toxicity Study.

Data for 4: PRG has been an approved product for pain management since 2005, and has been prescribed to, and taken by, over 16 million people, thus the existing clinical database is large (Lyrica website FAQ). No evidence of detectible levels of systemic exposure have been found after ophthalmic application of PRG¹², which can favorably impact the safety profile. Toxicokinetic analysis after high doses will be performed in this study for definitive data on levels of systemic exposure. We have validated a bioanalytical method to evaluate the concentrations of PRG with Ultra High Performance Liquid Chromatography-Tandem Mass Spectrometry¹² following the method of Pauly et al⁴⁰, with modifications including the use of a guard column. The level of quantitation (LOQ) using this HPLC method is ˜2.5 μg/mL.

Repeat dose safety studies have been conducted in house and has not observed any adverse events. Determining the no-observed-adverse-event-level (NOAEL) is an important part of the non-clinical risk assessment and will help determine the maximum recommended starting dose in our initial clinical trials. Increasing the dose (by increasing the concentration and increasing the number of applications per day) is necessary to determine the NOAEL and identify potential adverse events that should be monitored in clinical trials. This study will predicate and inform the doses used in the long term GLP repeat dose toxicity study.

TABLE 5
Animal distribution for toxicity study.

| | Total # animals | Treatment | Dose level |
|---|---|---|---|
| Group | M/F | both eyes | both eyes |
| 1 | 3/3 | Vehicle | 0 |
| 2 | 3/3 | Test Article | 0.6% |
| 3 | 3/3 | Test Article | 1.8% |
| 4 | 3/3 | Test Article | 6.0% |

Non-Limiting, Exemplary Research Design and Methods A standard ophthalmic toxicity study design will be followed in male and female Dutch belted rabbits. A low dose (efficacy dose), mid dose (3-times efficacy dose) and high dose (10-times efficacy dose) will be studied to assess the therapeutic window for our PRG ME. Animals will be acclimated to IOP measurements prior to collecting data.

Acclimation will include IOP measurement of both eyes in the AM and PM 5 days per week for 2 weeks. Animals will be examined twice daily for mortality checks and daily for cage side observations. Detailed clinical observations including body weights will be conducted weekly. Animals will be divided into 4 groups, and dosed according to Table 5 for 4 weeks. The following endpoints will be collected:

Gross ocular observations: Gross ocular observations will be obtained using the modified draize scoring system and will be conducted pre-study and once weekly approximately 0.5 hr following the last daily application.

Ocular Examination (OE): OEs will be performed by a board-certified veterinary ophthalmologist using slit lamp biomicroscopy and indirect ophthalmoscopy. Both eyes will be evaluated using the modified Hackett-McDonald scoring system. Exams will be performed once pre-study, and on Days 1, 8, 15, 22, and prior to necropsy approximately 0.5 hr following the last daily application.

Intraocular Pressure (IOP): IOPs will be obtained using rebound tonometer to evaluate pharmacologic effect in both eyes. IOP will be measured pre-study and once during week 1, once during week 2, and prior to necropsy.

Pachymetry: Pachymetry readings will be made in both eyes from the central cornea and will be conducted pre-study and prior to necropsy.

Corneal Specular Microscopy: Corneal specular microscopy will be evaluated in both eyes of anesthetized animals and will include the standard numerical evaluation of corneal endothelial cells. Evaluations will be conducted pre-study and prior to necropsy.

Electroretinography (ERG): Full field dark adapted electroretinography testing will be performed in both eyes of anesthetized animals. Evaluations will be conducted pre-study and prior to necropsy.

Clinical Pathology: Clinical Pathology assessments will be conducted on blood collected once pre-study, and prior to necropsy. Urine will be collected at necropsy. Parameters evaluated will be the standard hematology, coagulation, serum chemistry, and uranalysis panels.

Toxicokinetics (TK): Test article concentrations in the plasma will be evaluated from blood collected on Day 1 and Day 29 (10, 30, and 90 min following the second daily application).

Gross Necropsy: A gross necropsy will be performed that includes a standard non-ocular tissue list. Organ weights will be collected. Ocular tissues that will be collected include: right eye with bulbar conjunctivae, eyelids, lacrimal glands, mandibular lymph nodes, nictitating membranes, Harderian glands, optic nerves, nasal turbinates, nasopharnyx, enucleated right eye with aqueous humor collection.

Tissue Concentrations: Test Article concentrations in the following ocular tissues will be collected: aqueous humor, cornea, iris/cilliary body, vitreous humor, retina, choroid/RPE.

Histopathology: Ocular and adnexal tissue from all animals will be evaluated microscopically, and three slides from each eye will be evaluated (paraffin embedded and H&E stain). Non-ocular tissues will be stored in fixative for future evaluation.

The studies aim to 1) gain insight and concurrence with the FDA regarding our plans for completing studies and 2) determine the NOAEL of our PRG ME in rabbits and confidently select the appropriate doses to be used in our subsequent experiments for the GLP long term repeat dose study in rabbits.

Performance Endpoints: The primary endpoints/success metrics of Test 3 will include: 1) concurrence on plans for completing studies, 2) the final study report for the 4-week study that includes determined NOAEL, and 3) recommended dose levels for our future long term GLP repeat dose toxicity study.

### REFERENCES CITED IN THIS EXAMPLE

1 Tham, Y.-C. et al. Global Prevalence of Glaucoma and Projections of Glaucoma Burden through 2040. Opthalmology 121, 2081-2090 (2014).
2 Allingham, R. R., Liu, Y. & Rhee, D. J. The genetics of primary open-angle glaucoma: A review. Experimental Eye Research 88, 837-844, doi:10.1016/j.exer.2008.11.003 (2009).
3 Gordon, M. et al. The Ocular Hypertension Treatment Study: baseline factors that predict the onset of primary open-angle glaucoma. Arch Ophthalmol 120, 714-720 (2002).
4 Libby, R., Gould, D., Anderson, M. & John, S. W. Complex genetics of glaucoma susceptibility. Ann Rev Genomics Hum Genet 6, 15-44 (2005).
5 Quigley, H. A. Glaucoma: macrocosm to microcosm: The Friedenwald Lecture. Invest Ophthalmol Vis Sci 46, 2662-2670 (2005).
6 Quigley, H. A. & Broman, A. T. The number of people with glaucoma worldwide in 2010 and 2020. Br J Ophthalmol 90, 262-267 (2006).
7 Armaly, M. et al. Biostatistical analysis of the collaborative glaucoma study. I. Summary report of the risk factors for glaucomatous visual-field defects. Arch Ophthalmol 98, 2163-2171 (1980).
8 Leske, M., Connell, A., Wu, S., Hyman, L. & Schachat, A. Risk factors for open-angle glaucoma. The Barbados Eye Study. Arch Ophthalmol 113, 918-924 (1995).
9 Sommer, A. et al. Relationship between intraocular pressure and primary open angle glaucoma among white and black Americans. Arch Ophthalmol 109, 1090-1095 (1991).
10 Chintalapudi, S. et al. Systems Genetics Identifies a Role for Cacna2d1 Regulation in Elevated Intraocular Pressure and Glaucoma Susceptibility. Nature Communications 8, 1755 (2017).
11 Klugbauer, N., Marais, E. & Hofmann, F. Calcium channel alpha2delta subunits: differential expression, function, and drug binding. Journal of Bioenergetics and Biomembranes 35, 639-647 (2003).
12 Ibrahim, M. et al. Once Daily Pregabalin Eye Drops for Management of Glaucoma. ACS Nano 13, 13728-13744 (2019).
13 Brubaker, R. in Glaucoma. Applied Pharmacology in Medical Treatment (eds S M Drance & A H Neufeld) 35-70 (Grune and Strat-ton, Inc., 1984).
14 Ryskamp, D. et al. TRPV4 regulates calcium homeostasis, cytoskeletal remodeling, conventional outflow and intraocular pressure in the mammalian eye. Sci Rep 6, 30583 (2016).
15 Chintalapudi, S. R. et al. Systems genetics identifies a role for Cacna2d1 regulation in elevated intraocular pressure and glaucoma susceptibility. Nat Commun 8, 1755 (2017).
16 Belliotti, T. et al. Structure-Activity Relationships of Pregabalin and Analogues That Target the α2-δ Protein. J. Med. Chem 48, 2294-2307 (2005).
17 Field, M. et al. Identification of the alpha2-delta-1 subunit of voltage-dependent calcium channels as a molecular target for pain mediating the analgesic actions of pregabalin. Proc Natl Acad Sci USA 103, 17537-17542 (2006).
18 Bonnet, U. & Scherbaum, N. How addictive are gabapentin and pregabalin? A systematic review. Eur Neuropsychopharmacol 27, 1185-1215 (2017).
19 Pfizer. labeling.pfizer.com/ShowLabeling.aspx?id=561#S6.1.
20 McElnea, E. et al. Oxidative stress, mitochondrial dysfunction and calcium overload in human lamina cribrosa cells from glaucoma donors. Mol Vis 17, 1182-1191 (2011).
21 Crish, S. & Calkins, D. Neurodegeneration in glaucoma: progression and calcium-dependent intracellular mechanisms Neuroscience 176, 1-11 (2011).
22 Payne, L., Slagle, T., Cheeks, L. & Green, K. Effect of Calcium Channel Blockers on Intraocular Pressure in Rabbits. Ophthal Res 22, 337-341 (1990).
23 Wiederholt, M., Thieme, H. & Stumpff, F. The regulation of trabecular meshwork and ciliary muscle contractility. Prog Retin Eye Res 19, 271-295 (2000).
24 Siegner, S., Netland, P., Schroeder, A. & Erickson, K. Effect of calcium channel blockers alone and in combination with antiglaucoma medications on intraocular pressure in the primate eye. J Glaucoma 9, 334-339 (2000).
25 Luksch, A. et al. Effect of nimodipine on ocular blood flow and colour contrast sensitivity in patients with normal tension glaucoma. Br J Ophthalmol 89, 21-25, doi:10.1136/bjo.2003.037671 (2005).
26 Jani, A., Goyal, R., Shah, G. & Mehta, A. Effect of calcium channel blockers on intraocular pressure in rabbits. Iranian J Pharm & Therap 4, 95-99 (2005).
27 Araie, M. & Mayama, C. Use of calcium channel blockers for glaucoma. Prog Retin Eye Res 30, 54-71 (2011).
28 Ganekal, S., Dorairaj, S., Jhanji, V. & Kudlu, K. Effect of Topical Calcium Channel Blockers on Intraocular Pressure in Steroid-induced Glaucoma. J. Curr Glaucoma Pract 8, 15-19 (2014).
29 Wittenborn, J. & Rein, D. in Prevent Blindness America (2013).
30 National_Eye_Institute. Glaucoma: NEI Looks Ahead, nei.nih.gov/health
31 U.S. Department of Health and Human Services (HHS), Food and Drug Administration (FDA), Center for Drug Evaluation and Research (CDER) & Center for Biologics Evaluation and Research (CBER). in The Federal Register Vol. 68 (2003).
32 Chen, H. et al. Hydrogel-thickened microemulsion for topical administration of drug molecule at an extremely low concentration. Int J Pharm 341, 78-84 (2007).
33 Dehghani, F. et al. Preparation, characterization and in-vivo evaluation of microemulsions containing tamoxifen citrate anti-cancer drug. Eur J Pharm Sci 96, 479-489 (2017).
34 Vukkum, P., Babu, J. & Muralikrishna, R. Stress degradation behavior of pregabalin, identification of degradation impurities and development of stability indicating UPLC method. Int J Pharm Sci Res 6, 2241-2257 (2015).
35 Kalam, M. A., Alshamsan, A., Aljuffali, I. A., Mishra, A. K. & Sultana, Y. Delivery of gatifloxacin using microemulsion as vehicle: formulation, evaluation, transcorneal permeation and aqueous humor drug determination. Drug Deliv 23, 896-907 (2016).
36 Ibrahim, M., Abd-Elgawad, A., Soliman, O. & Jablonski, M. Natural Bioadhesive Biodegradable Nanoparticle-Based Topical Ophthalmic Formulations for Management of Glaucoma. Transl Vis Sci Technol 4, 12 (2015).
37 Ibrahim, M., Abd-Elgawad, A., Soliman, O. & Jablonski, M. Stability and Ocular Pharmacokinetics of Celecoxib-Loaded Nanoparticles Topical Ophthalmic Formulations. J Pharm Sci 105, 3691-3701 (2016).
38 Mayol, L., Quaglia, F., Borzacchiello, A., Ambrosio, L. & La Rotonda, M. I. A novel poloxamers/hyaluronic acid in situ forming hydrogel for drug delivery: rheological, mucoadhesive and in vitro release properties. Eur J Pharm Biopharm 70, 199-206, doi:10.1016/j.ejpb.2008.04.025 (2008).
39 Tayel, S. A., El-Nabarawi, M. A., Tadros, M. I. & Abd-Elsalam, W. H. Promising ion-sensitive in situ ocular nanoemulsion gels of terbinafine hydrochloride: design, in vitro characterization and in vivo estimation of the ocular irritation and drug pharmacokinetics in the aqueous humor of rabbits. Int J Pharm 443, 293-305, doi:10.1016/j.ijpharm.2012.12.049 (2013).
40 Pauly, C., Yegles, M. & Schneider, S. Pregabalin Determination in Hair by Ultra-High-Performance Liquid Chromatography-Tandem Mass Spectrometry. J Anal Toxic 37, 676-679 (2013).

### Example 5

Dutch belted rabbits were dosed with the topical pregabalin microemulsion for 60 days. Other rabbits were naïve and received no treatment. Rabbits that received the pregabalin microemulsion treatment had protection of their optic nerves.

### Example 6

Validating the Molecular Mechanism of Action of the Pharmaceutical Pregabalin on its Cognate Receptors CACNA2D1 and CACNA2D2

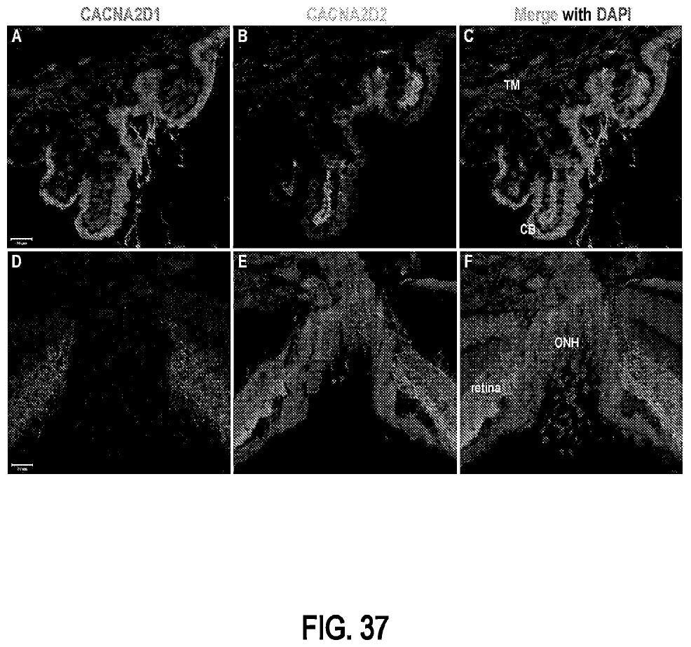

The FDA-approved drug pregabalin can lower IOP [1, 2]. To determine the mechanism of action by which pregabalin lowers IOP, we needed to identify the substrate to which pregabalin binds as well as determine if that substrate is present in regions of the eye responsible for IOP production and regulation. A target of pregabalin can be calcium channel, voltage-dependent subunits, α2δ1 (aka CACNA2D1). To validate that CACNA2D1 can be present in the eye and localized to regions that affect IOP, we performed fluorescent immunohistochemistry (fIHC), which demonstrated that CACNA2D1 is localized to regions which can modulate IOP, for example the trabecular meshwork and ciliary body (FIG. 37 panels A-C). Moreover, pregabalin, when delivered topically in a aqueous solution and an extended release microemulsion [1, 2] lowers IOP in a dose dependent manner. Through further studies of biodistribution of pregabalin in the eye after 21 days of dosing [2], we were surprised to measure high levels of pregabalin in the optic nerve; however, CACNA2D1 was present minimally, if at all, in the optic nerve. Because pregabalin binds with similar affinity to CACNA2D2, a second delta subunit, without wishing to be bound by theory, pregabalin can have a different function in the optic nerve via interactions with CACNA2D2. Using immunohistochemistry and confocal microscopy, we show that CACNA2D2 localizes to the optic nerve (FIG. 37 panels D-F), providing an explanation for the elevated levels in the optic nerve after topical dosing with pregabalin [2].

### REFERENCES CITED IN THIS EXAMPLE

1. Chintalapudi, S., Maria, D., Wang, X., Cooke Bailey, J., NEIGHBORHOOD Consortium, International Glaucoma Genetics Consortium, Hysi, P., Wiggs, J., Williams, R. & Jablonski, M. (2017) Systems genetics identifies a role for Cacna2d1 regulation in elevated intraocular pressure and glaucoma susceptibility Nat Commun. 8, 1755.
2. Ibrahim, M., Maria, D., Wang, X., Mishra, S., Guragain, D. & Jablonski, M. (2019) Once Daily Pregabalin Eye Drops for Management of Glaucoma, ACS Nano. 13, 13728-13744.

### Example 7

Inherited retinal degenerations (IRD) can affect photoreceptors in the retina, and can cause them to die and subjects can lose their vision because of this. In contrast to glaucoma (that can affect older people), IRDs affect can younger people (for example <30 years old). Some forms of IRDs can affect teens to young adults. These can be called “retinitis pigmentosa”. Other forms can affect young children. These can be called “Leber congenital amaurosis”. There are many genes that can cause these diseases. Other forms of these diseases can be called “cone rod or rod cone dystrophies”.

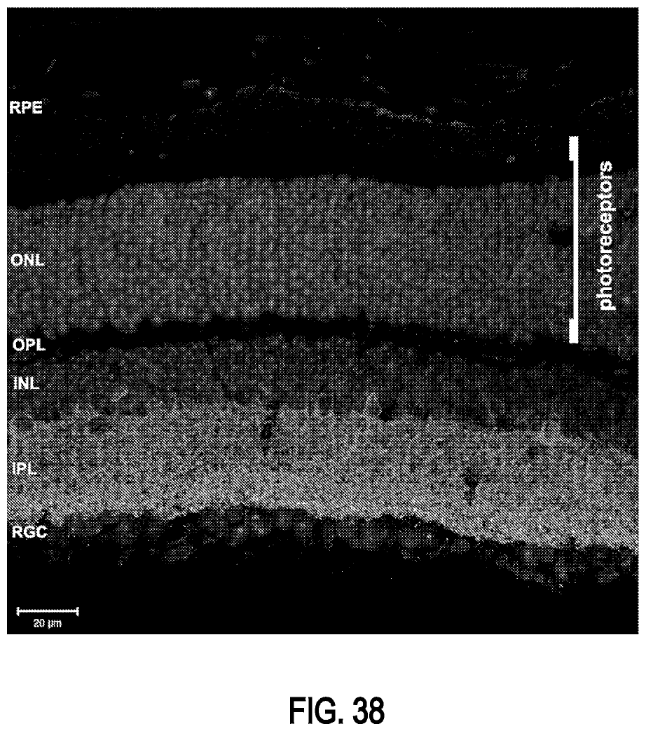

We used immunohistochemistry to determine that CACNA2D1, one of the targets of pregabalin, can be localized to photoreceptors in the retina. As shown in FIG. 38:
RPE=retinal pigment epithelium; ONL=outer nuclear layer; OPL=outer plexiform layer; INL=inner nuclear layer; IPL=inner plexiform layer; RGC=retinal ganglion cell layer
red shows where CACNA2D1 is localized
CACNA2D1 is localized to photoreceptors and throughout the inner retina
green shows where CACNA2D2 is localized
CACNA2D2 is localized throughout the inner retina
both CACNA2D1 & CACNA2D2 bind pregabalin
given that CACNA2D1 is expressed by photoreceptors, it can act as a neuroprotectant there.

### Example 8

The studies described herein can provide therapies for: eye injury or visual dysfunction as related to a military-relevant traumatic event; and stabilization, and treatment of eye injuries in austere environments and prolonged field care settings.

Without wishing to be bound by theory, we will validate a rabbit model of ocular blast injury (OBI), which possesses an eye approximating the size of the human eye thereby facilitating the applicability of our outcomes to the clinic, and determine the efficacy and mechanism of action of a topical formulation of pregabalin—a molecule we identified as a neuroprotectant in murine and rabbit models of glaucomatous retinal ganglion cell (RGC) and optic nerve (ON) damage—in our rabbit OBI model.

Soldiers younger than 30 years old can experience many years of impaired vision after a blast injury¹. Therapies that can preserve vision are of interest. Without wishing to be bound by theory, pregabalin microemulsion (ME) is validated as a therapeutic for blast-OBI. Without wishing to be bound by theory, a new model of ocular trauma is validated herein.

Background

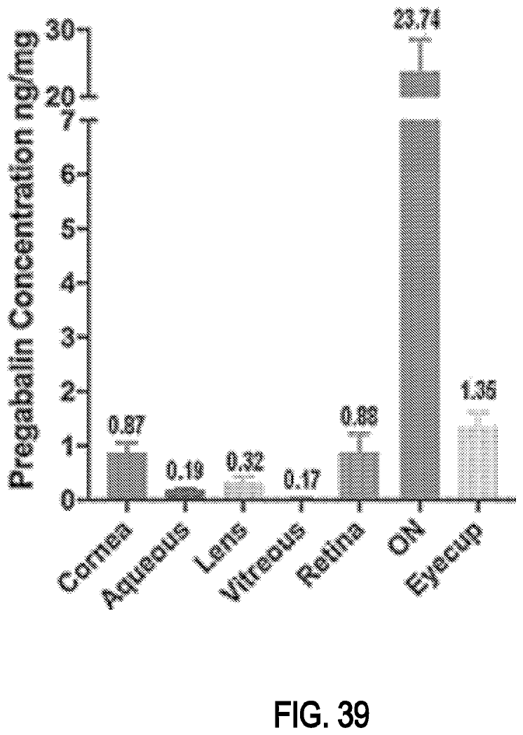

There is an unmet need for therapies & drug delivery systems for vision loss after percussive injury. Improvements in body armor have reduced the incidence of fatal traumas to warriors; however, ocular injuries remain a cause of morbidity. Complications can comprise RGC (retinal ganglion cells) and ON (ocular nerve) damage and loss of visual field. Therapies that treat these injuries have been lacking. There is a need to deliver neuroprotectants noninvasively to the posterior segment of the eye. Embodiments to address both of these unmet needs is described herein; we have determined that pregabalin provides neuroprotection to RGCs and the ON in glaucoma, and have a engineered a topical formulation to facilitate entry of the drug into the eye. We had identified pregabalin as a modulator of intraocular pressure (IOP). Surprisingly, we have also discovered that it is a neuroprotectant in murine and rabbit models of glaucomatous RGC and ON damage. Our formulation is a bioadhesive multi-layered microemulsion (ME)-based topical eye drop that provides sustained release of pregabalin for >24 hours from a single dose². The advantages of our ME include ease of preparation, 100% encapsulation efficiency, increased drug corneal contact time due to its bioadhesiveness, controlled drug release due to its multilayered structure, and sustained high corneal permeability². The net result of these characteristics is that pregabalin can enter the eye and diffuse to the retina and ON (FIG. 39). Our ME is also safe and well tolerated in the eye^{2,3}.

There is an unmet need for a preclinical OBI model with large eyes to facilitate the transition to human studies. For nearly 10 years, we have used mice with great success as preclinical models of OBI and traumatic brain injury (TBI) and characterized the central and peripheral visual system injury and deficits⁴⁻⁹. While mice are valuable for determining mechanisms of injury and early-stage assessment of plausible neuroprotective molecules, a preclinical model with eyes that approximate the size of human eyes is a requirement for moving a therapeutic into human clinical trials. To fulfill this need, we will validate our rabbit model of OBI and use it as a platform for evaluating our pregabalin ME as a neuroprotective therapy for OBI.

Aims and Approaches:

Without wishing to be bound by theory, neuroprotective therapies to preserve vision in wounded soldiers affected by ocular blast injury are described herein. We can evaluate the efficacy of our topical pregabalin ME formulation as a therapeutic for OBI. Without wishing to be bound by theory, pregabalin will mitigate dysfunction and loss of RGC axons by attenuating an increase in intracellular free calcium levels [Ca²⁺.

Aim 1: We will validate our preclinical rabbit model of OBI.

Aim 2: We will validate that formulated pregabalin will offer neuroprotection to RGCs and the ON after OBI in the rabbit by regulating the concentration of intracellular free calcium [Ca²⁺]ᵢ levels in RGCs and their axons.

Aim 1: We will validate our preclinical rabbit model of OBI. This will allow us to advance the rabbit as a validated preclinical model of OBI that will facilitate the progression of pregabalin and other drugs to clinical trials in humans.

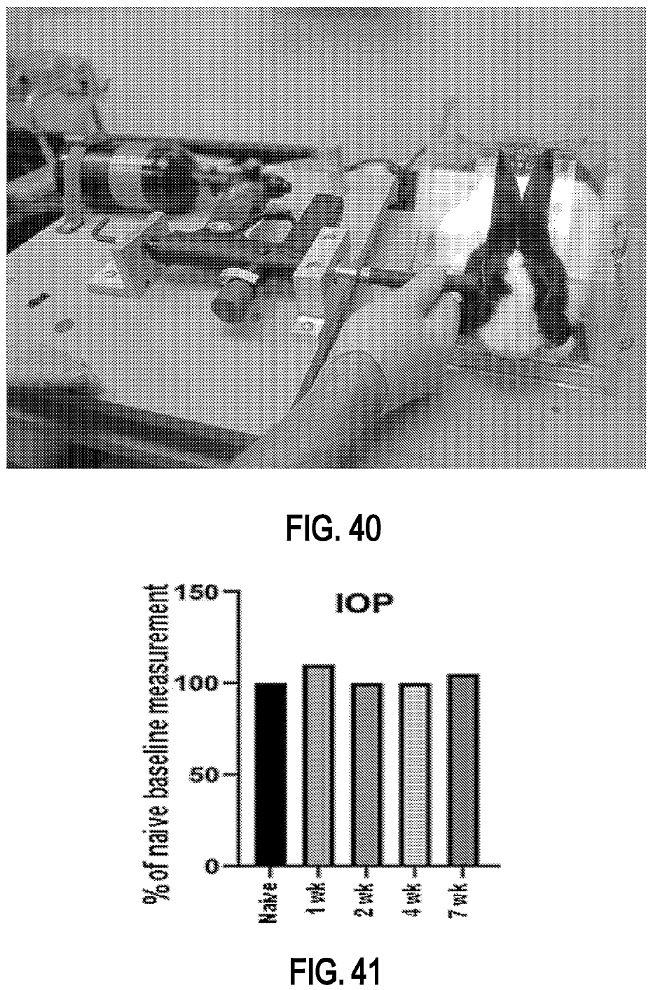

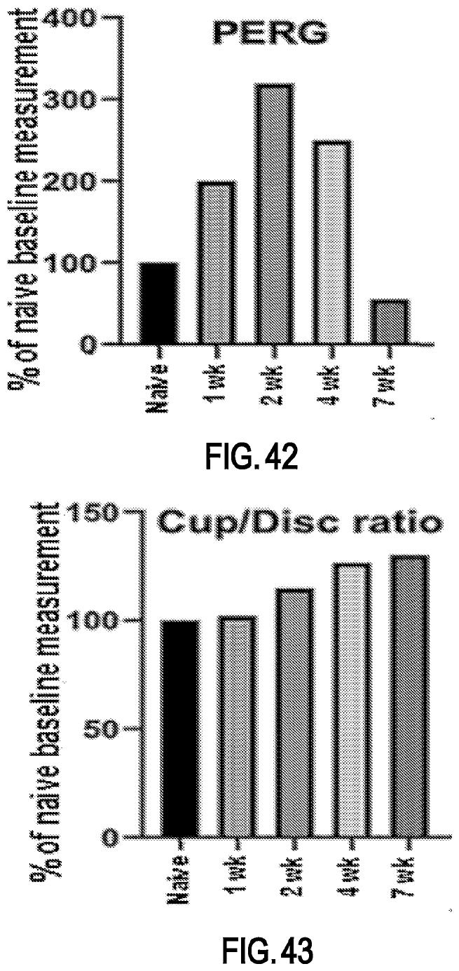

Data: We evaluated injury after a single blast of 40, 45, or 50 psi to the eyes of Dutch Belted (DB) rabbits (FIG. 40). 50 psi is a pressure that can produce consistent and documentable functional injury to RGCs and ON. IOP was constant during the post-OBI period (FIG. 41). Pattern ERG (PERG) amplitudes, reflective of RGC function, transiently increased during the first two weeks post-injury (FIG. 42), and, without wishing to be bound by theory, this can or cannot be due to the release of protective cytokines, as has been demonstrated in instances of mild injury^{10,11}, but then declined by 7 weeks. Despite the temporary increase in RGC function, we documented a steady increase in the cup to disc ratio (FIG. 43), which is reflective of a loss RGC axons as they exit the eye at the optic nervehead (ONH). Histopathology and assessment of injury markers can be performed after sacrifice at 10 weeks post-injury.

Non-limiting exemplary methods: Model selection and injury paradigm. Based on power calculations, we can enroll 36 rabbits (˜3 mo of age; 6M, 6F per group; 2 OBI groups & 1 control; 3 timepoints; Covance). To produce OBI, anesthetized rabbits will be secured in a stereotaxic frame and the tip of our blast cannon positioned 5 mm from the cornea (FIG. 40). We will generate and characterize two OBI models: mild injury due to a single blast of 50 psi in one group (FIGS. 41-43); and moderate injury due to a series of five repeated blast pressures at 50 psi with a 60 second delay between blasts in another, as we have done in our mouse studies⁴⁻⁷. Both eyes of each rabbit will receive the same injury within minutes of each other. A control group will be placed in the blast apparatus, but receive no injury. Baseline measurements on naïve rabbits will be taken prior to the beginning of the study. The damaging effects of OBI will be evaluated post-blast using the following assessments:

Clinical examinations at pre-injury baseline and 2, 6 and 10 weeks post-OBI: IOP measurement; scotopic full field electroretinogram (ERG) and pattern ERG (PERG) testing, which can be indicators of photoreceptor/biopolar cell and RGC function, respectively; fundus imaging with cup-to-disc ratio calculations; and OCT imaging to evaluate changes in retinal thickness using our published methods^{2,12-15}.

Laboratory examinations at pre-injury baseline and 2, 6 and 10 weeks post-OBI. Two rabbits (1M, 1F) will be euthanized at each time point and the following studies will be performed: morphology of the retina, ON, and optic tract; immunohistochemical assessment of microglia status; determination of the presence of injured axon bulbs in the optic tract, and histological assessment of ON damage using our methods^{4,5}.

Statistical analyses: Statistical differences will be analyzed by one-way ANOVA followed by Tukey-Kramer multiple comparisons test (in the event of a significant F-test) using Prism statistical software. Differences in responses of males and females will be determined. If no differences are identified, data will be pooled.

Without wishing to be bound by theory, we can include the characterization of two models of OBI in the rabbit.

Aim 2: Validate that formulated pregabalin will offer neuroprotection to RGCs and the ON after OBI in the rabbit by regulating the concentration of intracellular calcium (Ca²⁺) levels in RGCs and their axons. We can validate the efficacy and mechanism of action of our formulated pregabalin as a neuroprotective agent in rabbits that suffered OBI. Without wishing to be bound by theory, delivery of a neuroprotective therapeutic via topical dosing can improve treatment options. Due to the nature of the dosing—a single daily eye drop—our formulation can be used in field hospitals, austere environments, and prolonged field care settings among others.

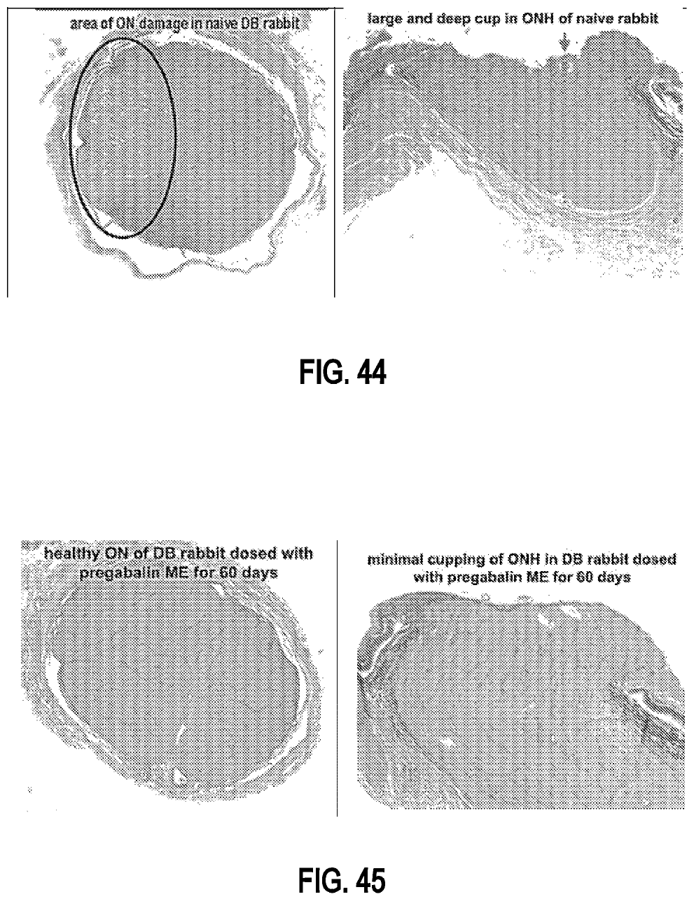

Data: DB rabbits can develop spontaneous age-related glaucoma manifest as ON damage and ON head cupping (FIG. 44). However, daily dosing with our pregabalin ME during the early phase of glaucomatous damage mitigated these structural defects (FIG. 45). We can validate evaluate our pregabalin ME as a plausible neuroprotectant for OBI. For example, we can use young rabbits (3 months old that have not yet developed age-related increases in IOP and ON damage) because this age can simulate the age of a soldier that suffered an OBI.

The mechanism of action by which pregabalin is neuroprotective to RGCs is unknown. Without wishing to be bound by theory, and given that it is a selective CACNA2D1 blocker¹⁶, pregabalin can attenuate the influx of ionic Ca²⁺ across the cell membrane of RGCs and their axons (FIG. 10).

Non-limiting, exemplary methods: Evaluation of pregabalin as a neuroprotectant. Based on power calculations, we will enroll 36 rabbits (˜3 mo of age; 6M, 6F per group; 2 OBI groups & 1 control; 3 timepoints; Covance). To validate the neuroprotective effect of our pregabalin ME in our mild and moderate OBI models (see herein) rabbits will be dosed beginning at 2 hours postinjury (to simulate the length of time that soldiers can experience between injury and initiation of treatment). Injured eyes will receive a 30 μl dose of pregabalin ME (0.6%²). Dosing will continue daily for 10 weeks. The neuroprotective potential of pregabalin ME will be evaluated using the same clinical and laboratory examinations outlined herein.

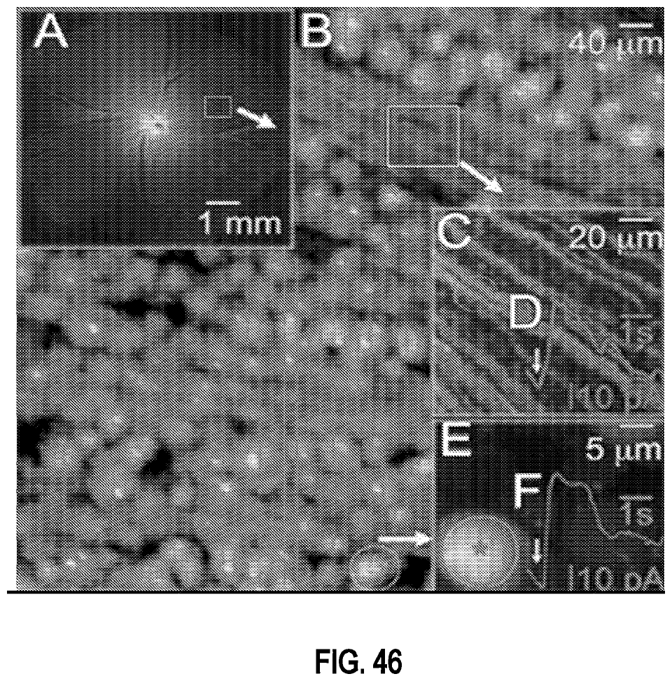

Evaluation of effect of pregabalin on [Ca²⁺]ᵢ. We will enroll 36 rabbits (˜3 mo of age; 2M, 2F per group; 2 OBI groups & 1 control; 3 timepoints; Covance). To validate that pregabalin mitigates the influx Ca²⁺ in our mild and moderate OBI models, we will measure Ca²⁺ signals mediated by voltage-gated Ca²⁺ channels (VGCCs) in RGC somas and their axons at the following times post-injury: 2 hr, 4 hr and 2 weeks, which will allow us to access both acute and chronic changes. To do so, eye cups from each condition will be loaded retrogradely with a mixture of a Ca²⁺-sensitive and insensitive dyes, allowing us to use a ratiometric approach to calculate the amplitude and duration of [Ca²⁺]ᵢ¹⁷. Cells will be depolarized by raising the extracellular potassium concentration to activate VGCCs^{18,19} via automated perfusion synchronized with imaging computer^{20,21}. The changes in [Ca²⁺]ᵢ will be acquired by placing regions of interest (ROIs) on specific RGC axon bundles and somata (FIG. 46 panels C & E). The Na⁺ channel blocker tetrodotoxin will be used to determine the extent to which Na⁺ channels contribute to the generation of Ca²⁺ transients. Fluorescent intensity values from ROIs will be averaged within an experimental condition, each considered an independent observation for statistical testing.

Without wishing to be bound by theory, injured eyes dosed with our topical pregabalin ME have fewer functional and histopathological abnormalities than those that are untreated. Without wishing to be bound by theory, OBI leads to an unregulated and elevated influx of ionic Ca²⁺ that can be mitigated by pregabalin. Without wishing to be bound by theory, a stable and low [Ca²⁺]ᵢ will be correlated with clinical and histopathological neuroprotection.

### REFERENCES CITED IN THIS EXAMPLE

1 Stallard, H. B. Retinal Detachment Due to War Trauma. Br. J Ophthalmol 7, 419-429 (1946).
2 Ibrahim, M. et al. Once Daily Pregabalin Eye Drops for Management of Glaucoma. ACS Nano 13, 13728-13744 (2019).
3 Ibrahim, M. et al. Enhanced Corneal Penetration of a Poorly Permeable Drug Using Bioadhesive Multiple Microemulsion Technology Pharmaceutics 12, 704, doi:doi: 10.3390/pharmaceutics12080704 (2020).
4 Guley, N. et al. A Novel Closed-Head Model of Mild Traumatic Brain Injury Using Focal Primary Overpressure Blast to the Cranium in Mice. J. Neurotrauma 33, 403-422 (2016).
5 Reiner, A. et al. Motor, Visual and Emotional Deficits in Mice after Closed-Head Mild Traumatic Brain Injury Are Alleviated by the Novel CB2 Inverse Agonist SMM-189. Int J Mol Sci 16, 758-787 (2015).
6 Honig, M. et al. Amelioration of visual deficits and visual system pathology after mild TBI via the cannabinoid Type-2 receptor inverse agonism of raloxifene. Exp Neurol 322, 113063 (2019).
7 Bu, W. et al. Mild traumatic brain injury produces neuron loss that can be rescued by modulating microglial activation using a CB2 receptor inverse agonist. Front Neurosci 10, 449 (2016).
8 Guley, N. et al. Amelioration of visual deficits and visual system pathology after mild TBI with the cannabinoid type-2 receptor inverse agonist SMM-189. Exp Eye Res 182, 109-124 (2019).
9 Liu, Y. et al. Abnormalities in dynamic brain activity caused by mild traumatic brain injury are partially rescued by the cannabinoid type-2 receptor inverse agonist SMM-189. eNeuro 18, 4 (2017).
10 Roth, S. Endogenous neuroprotection in the retina. Brain Res Bull 62, 461-466 (2004).
11 Simon, G., Hovda, D., Harris, N., Gomerz-Pinilla, F. & Goldberg, R. Traumatic brain injury induced neuroprotection of retinal ganglion cells to optic nerve crush J Neurotrauma 23, 1072-1082 (2006).
12 Ibrahim, M., Abd-Elgawad, A., Soliman, O. & Jablonski, M. Novel topical ophthalmic formulations for management of glaucoma. Pharm Res 30, 2818-2831 (2013).
13 Swaminathan, S. et al. Novel Endogenous Glycan Therapy For Retinal Diseases: Safety, In Vitro Stability, Ocular Pharmacokinetic Modeling and Bio-distribution. AAPS Journal 16, 311-312 (2014).
14 Ibrahim, M., Abd-Elgawad, A., Soliman, O. & Jablonski, M. Natural Bioadhesive Biodegradable Nanoparticle-Based Topical Ophthalmic Formulations for Management of Glaucoma. Transl Vis Sci Technol 4, 12 (2015).
15 Ibrahim, M., Abd-Elgawad, A., Soliman, O. & Jablonski, M. Natural bioadhesive biodegradable nanoparticles-based topical ophthalmic formulations for sustained celecoxib release: In vitro Study. Pharm Technol & Drug Res 2, 7 (2013).
16 Chintalapudi, S. R. et al. Systems genetics identifies a role for Cacna2d1 regulation in elevated intraocular pressure and glaucoma susceptibility. Nat Commun 8, 1755 (2017).
17 Van Hook, M. J. & Thoreson, W. B. Weak endogenous Ca2+ buffering supports sustained synaptic transmission by distinct mechanisms in rod and cone photoreceptors in salamander retina. Physiol Rep 3, doi: 10.14814/phy2.12567 (2015).
18 Vaithianathan, T., Akmentin, W., Henry, D. & Matthews, G. The ribbon-associated protein C-terminal-binding protein 1 is not essential for the structure and function of retinal ribbon synapses. Mol Vis 19, 917-926 (2013).
19 Vaithianathan, T. & Matthews, G. Visualizing synaptic vesicle turnover and pool refilling driven by calcium nanodomains at presynaptic active zones of ribbon synapses. Proc Natl Acad Sci USA 111, 8655-8660, doi:10.1073/pnas.1323962111 (2014).
20 Vaithianathan, T., Henry, D., Akmentin, W. & Matthews, G. Nanoscale dynamics of synaptic vesicle trafficking and fusion at the presynaptic active zone. Elife 5, doi:10.7554/eLife.13245 (2016).
21 Vaithianathan, T., Wollmuth, L. P., Henry, D., Zenisek, D. & Matthews, G. Tracking Newly Released Synaptic Vesicle Proteins at Ribbon Active Zones. iScience 17, 10-23, doi:10.1016/j.isci.2019.06.015 (2019).

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1. A method of treating glaucoma in a subject in need thereof, the method comprising administering to the subject an effective amount of an ocular composition comprising pregabalin, wherein the glaucoma is normal tension glaucoma.

2. (canceled)

3. (canceled)

4. A method of preventing further ocular neurodegeneration in a subject in need thereof, the method comprising administering to the subject an effective amount of an ocular composition comprising pregabalin (PRG).

5.-8. (canceled)

9. The method of claim 1, wherein the ocular composition contains about 0.001% to about 1.2% of pregabalin.

10. (canceled)

11. The method of claim 1, wherein the composition is administered topically.

12. (canceled)

13. (canceled)

14. The method of claim 1, wherein the composition is administered once per day.

15. The method of claim 1, wherein the composition comprises a microemulsion formulation.

16. (canceled)

17. The method of claim 15, wherein the microemulsion comprises a water-in-oil-in-water (W₁/O/W₂) multiple microemulsion.

18. The method of claim 17, wherein the W₁/O/W₂ multiple microemulsion comprises:
an internal phase (W₁) comprising an aqueous solution encompassed within an internal emulsifier;
a continuous oil phase (O) encompassing the internal phase and encompassed within an external emulsifier; and
an external aqueous phase (W₂) surrounding the external emulsifier.

19. The method of claim 18, wherein the intermediate oil phase comprises an internal emulsifier.

20. The method of claim 19, wherein the internal emulsifier comprises a surfactant with a hydrophobic-lipophobic balance (HLB) value of 3-7.

21. The method of claim 18, wherein the internal emulsifier comprises lecithin, propylene glycol monocaproate, propylene glycol monocaprylate, propylene glycol monocaprate, propylene glycol monolaurate, propylene glycol monostearate, propylene glycol monopalmitate, polyethylene glycol lauryl ether, polyethylene glycol oleyl ether, polyethylene glycol hexadecyl ether, sorbitan monopalmitate, sorbitan monostearate, sorbitan monooleate, sorbitan monolaurate, transcutol P, gelucire 50/13 (mixture of PEG (MW 1500) mono-, di-, tri-esters of stearic acid), gelucire 44/14 (mixture of PEG (MW 1500) mono-, di-, tri-esters of lauric acid), gelucire 43/01 (mixture of PEG (MW 1500) mono-, di-, tri-esters of fatty acids C₈-C₁₈), any PEG mono-, di- and/or tri-esters of any fatty acid, egg lecithin, phosphatidylcholine, phosphatidylethanolamine, phosphatidylinositol, tocopherol or any other phospholipid, or a combination thereof.

22. (canceled)

23. The method of claim 18, wherein the external aqueous phase comprises a mucoadhesive polymer.

24.-29. (canceled)

30. The method of claim 1, wherein the ocular composition is administered to a subject once daily, twice daily, thrice daily, or once weekly.

31. The method of claim 18, wherein the external emulsifier comprises a surfactant with a HLB value of 10-16.

32. The method of claim 23, wherein the mucoadhesive polymer is selected from a polyacrylic acid, alginic acid or a salt thereof, chitosan, dextran, pectin, gelatin polyvinylpyrrolidone, N-methylpyrrolidone, hyaluronic acid or a salt thereof, gellan gum, xanthan gum, agar, glycocholic acid or a salt thereof, a derivative of the foregoing, or a combination thereof.

33. The method of claim 18, wherein the external aqueous phase further comprises pregabalin.

34. The method of claim 18, wherein:
the internal emulsifier comprises a surfactant with a HLB value of 3-7;
the external emulsifier comprises a surfactant with a HLB value of 10-16; and
the external aqueous phase comprises pregabalin.

35. The method of claim 18, wherein:
the internal emulsifier comprises polyethylene glycol oleyl ether and soybean lecithin;
the continuous oil phase comprises medium-chain triglycerides of caprylic (C8) and capric (C10) acids;
the external emulsifier comprises polyethylene glycol and macrogol glycerol ricinoleate; and
the external aqueous phase comprises pregabalin and a mucoadhesive polymer comprising chitosan or a derivative thereof.

36. The method of claim 1, wherein the composition prevents ocular neurodegeneration by preventing degeneration of axons in the optic nerve.

37. The method of claim 36, wherein the composition prevents degeneration of axons in the optic nerve in the absence of a reduction in intraocular pressure.

38. The method of claim 1, wherein the composition prevents ocular neurodegeneration by preventing death/dysfunction of retinal ganglion cells (RGCs).

39. The method of claim 1, wherein the composition prevents ocular degeneration by preventing death/dysfunction of RGC axons in the optic nerve.

40. A method for providing neuroprotection to the eye of a subject by preventing death or dysfunction of retinal ganglion cells (RGCs) of the eye, comprising:
contacting a plurality of RGCs of the subject with an inhibitor of the Calcium Voltage-Gated Channel Auxiliary Subunit Alpha2/Delta1 (CACNA2D2) protein.

41. The method of claim 40, wherein the inhibitor of the CACNA2D2 protein is pregabalin.

42. The method of claim 40, wherein the patient has glaucoma.

43. The method of claim 42, wherein the glaucoma is normal tension glaucoma.

44. The method of claim 40, wherein the patient does not have glaucoma.

45. A method for inhibiting the Calcium Voltage-Gated Channel Auxiliary Subunit Alpha2/Delta1 (CACNA2D2) protein in the eye of a human patient, comprising contacting the eye of the patient with pregabalin.

46. The method of claim 45, wherein the patient has glaucoma.

47. The method of claim 45, wherein the CACNA2D2 protein is located in the optic nerve of the patient.
